SEQUENCE LISTING (246 sequences)

SEQ ID NO: 1 - DNA, 369 bp, Artificial Sequence
  Synthetic
ctatataagcagagctcgtttagtgaaccgtcagatcgcctggagacgccatccacgctgttttgacctccatagaagatctagaagcaaaagacggcatacgaccatgctgctgctgctgctgctgctgggcctgaggctacagctctccctgggcggatccacgtcttcttaattgaagacacggttaattcggactgtagaactctgaacctgtcggtggtcgccgtatcattgtcgacaatcaacctctggattacaaaatttgtgaaagattgactggtattcttaactatgttgctccttttacgctatgtggatacgctgctttaatgcctttgtatcatgctattgcttcccgtatggctt

SEQ ID NO: 2 - DNA, 23 bp, Artificial Sequence
  Synthetic
tgaaccgtcagatcgcctggaga

SEQ ID NO: 3 - DNA, 23 bp, Artificial Sequence
  Synthetic
acgctgttttgacctccatagaa

SEQ ID NO: 4 - DNA, 22 bp, Artificial Sequence
  Synthetic
caagcaagaagacggcatacga

SEQ ID NO: 5 - DNA, 28 bp, Artificial Sequence
  Synthetic
aagcctgacatcttgagacttggacagc

SEQ ID NO: 6 - DNA, 22 bp, Artificial Sequence
  Synthetic
acagccaccagcggcatagtaa

SEQ ID NO: 7 - DNA, 27 bp, Artificial Sequence
  Synthetic
aactgaccataagaattgatacaacga

SEQ ID NO: 8 - DNA, 23 bp, Artificial Sequence
  Synthetic
aatgcgatacacctatgcgacga

SEQ ID NO: 9 - DNA, 201 bp, Artificial Sequence
  Synthetic
tctagaagcaaaagacggcatacgaccatgctgctgctgctgctgctgctgggcctgaggctacagctctccctgggcggatcccgtagctcctctcgcactccgtccgataagccggttgctcatgtagttgctaaccctcagggttaattcggactgtagaactctgaacctgtcggtggtcgccgtatcattgtcgac

SEQ ID NO: 10 - DNA, 29 bp, Artificial Sequence
  Synthetic
ccaattaagcctgacatcttgagacttga

SEQ ID NO: 11 - DNA, 28 bp, Artificial Sequence
  Synthetic
aagcctgacatcttgagacttggacagc

SEQ ID NO: 12 - DNA, 318 bp, Artificial Sequence
  Synthetic
tctagaagcaaaagacggcatacgaccatgctgctgctgctgctgctgctgggcctgaggctacagctctccctgggcggatccggcggtcgtatgaaacagctggaagataaaattgaagaactgctgtctaaaatctatcatctggaaaatgaaattgcgcgtctgaaaaaactgattggtgaacgtggcggagcatcccgtagctcctctcgcactccgtccgataagccggttgctcatgtagttgctaaccctcagggttaattcggactgtagaactctgaacctgtcggtggtcgccgtatcattgtcgac

SEQ ID NO: 13 - DNA, 318 bp, Artificial Sequence
  Synthetic
tctagaagcaaaagacggcatacgaccatgctgctgctgctgctgctgctgggcctgaggctacagctctccctgggcggatccggcggtcgtatgaaacagattgaagataaaattgaagaaattctgtctaaaatctatcatattgaaaatgaaattgcgcgtattaaaaaactgattggtgaacgtggcggagcatcccgtagctcctctcgcactccgtccgataagccggttgctcatgtagttgctaaccctcagggttaattcggactgtagaactctgaacctgtcggtggtcgccgtatcattgtcgac

SEQ ID NO: 14 - DNA, 273 bp, Artificial Sequence
  Synthetic
tctagaagcaaaagacggcatacgaccatgctgctgctgctgctgctgctgggcctgaggctacagctctccctgggcggatccatgggcgagttcttgatcgtgaagtcaggggcatcccgtagctcctctcgcactccgtccgataagccggttgctcatgtagttgctaaccctcagggttgtgaattccttatcgtcaaatcagggcctcctggttaattcggactgtagaactctgaacctgtcggtggtcgccgtatcattgtcgac

SEQ ID NO: 15 - DNA, 29 bp, Artificial Sequence
  Synthetic
ccaacacttaaggaatagcagtttagtcc

SEQ ID NO: 16 - DNA, 348 bp, Artificial Sequence
  Synthetic
tctagaagcaaaagacggcatacgaccatgctgctgctgctgctgctgctgggcctgaggctacagctctccctgggcggatcccgtagctcctctcgcactccgtccgataagccggttgctcatgtagttgctaaccctcagggttcggactgtagaactctgaacctgtcggtggtcgccgtatcattagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggtcgac

SEQ ID NO: 17 - DNA, 87 bp, Artificial Sequence
  Synthetic
acgaagacggatcccgtagctcctctcgcactccgtccgataagccggttgctcatgtagttgctaaccctcagggttcggtcttct

SEQ ID NO: 18 - DNA, 24 bp, Artificial Sequence
  Synthetic
agcacaatcaagacgaagacggat

SEQ ID NO: 19 - DNA, 23 bp, Artificial Sequence
  Synthetic
caagccagaagaggcatactgca

SEQ ID NO: 20 - protein, 50 aa, Artificial Sequence
  Synthetic
TQFGNVPWYSEACSSTLATTYSSGNQNEKQIVTTDLRQKCTESHTGTSAS

SEQ ID NO: 21 - protein, 50 aa, Artificial Sequence; identical to SEQ ID NO: 20
  Synthetic

SEQ ID NO: 22 - protein, 50 aa, Artificial Sequence
  Synthetic
QVLIQHLRGLQKGRSTERNVSVEALASALQLLAREQQSTGRVGRSLPTED

SEQ ID NO: 23 - protein, 31 aa, Artificial Sequence
  Synthetic
QVLIQHLRGLQKGRSTERNVSRVGRSLPTED

SEQ ID NO: 24 - protein, 50 aa, Artificial Sequence
  Synthetic
LLAREQQSTGRVGRSLPTEDCENEKEQAVHNVVQLLPGVGTFYNLGTALY

SEQ ID NO: 25 - protein, 50 aa, Artificial Sequence
  Synthetic
DSDQDQDGDGHQDSQDNCPTVPNSAQEDSDHDGQGDACDDDDDNDGVPDS

SEQ ID NO: 26 - protein, 20 aa, Artificial Sequence
  Synthetic
HQDSQDNCPTVPNSAQEDSD

SEQ ID NO: 27 - protein, 20 aa, Artificial Sequence
  Synthetic
RVGRSLPTEDCENEKEQAVH

SEQ ID NO: 28 - protein, 20 aa, Artificial Sequence
  Synthetic
DYMGWMDFGRRSAEEYEYPS

SEQ ID NO: 29 - protein, 21 aa, Artificial Sequence
  Synthetic
MRSLSVLALLLLLLLAPASAA

SEQ ID NO: 30 - protein, 10 aa, Artificial Sequence
  Synthetic
GGGSGGGSGG

SEQ ID NO: 31 - DNA, 359 bp, Artificial Sequence
  Synthetic
gcagagctcgtttagtgaaccgtcagatcgcctggagacgccatccacgctgttttgacctccatagaagattctagaagcaaaagacggcatacgagatcaccatgcgcagcctgagcgtgctggccctgctgctgctcctgctcctggcccctgcttctgccgctacgtcttcagaattcagaagacacggttcgggaggctcagggtcaggtcgaatgaagcaaatcgaggacaagttggaggagatcttgagcaagttgtaccacatcgagaacgaactagcgcgaatcaagaagttgttgggcgagcgaggatcctgagtgtcggtggtcgccgtatcatcttcgaatgtcgac

SEQ ID NO: 32 - DNA, 23 bp, Artificial Sequence
  Synthetic
caagcagaagacggcatacgaga

SEQ ID NO: 33 - DNA, 24 bp, Artificial Sequence
  Synthetic
ccaagccctccgagtcccagtcca

SEQ ID NO: 34 - protein, 21 aa, Artificial Sequence
  Synthetic
MRSLSVLALLLLLLLAPASAA

SEQ ID NO: 35 - protein, 43 aa, Artificial Sequence
  Synthetic
GSGGSGSGRMKQIEDKLEEILSKLYHIENELARIKKLLGERGS

SEQ ID NO: 36 - DNA, 304 bp, Artificial Sequence
  Synthetic
gcagagctcgtttagtgaaccgtcagatcgcctggagacgccatccacgctgttttgacctccatagaagattctagaagcaaaagacggcatacgagatcaccatgggcgctacgtcttcagattcagaagacacggttcgggaggctcagggtcaggtcgaatgaagcaaatcgaggacaagttggaggagatcttgagcaagttgtaccacatcgagaacgaactagcgcgaatcaagaagttgttgggcgagcgaggatcctgagtgtcggtggtcgccgtatcatcttcgaatgtcgac

SEQ ID NO: 37 - DNA, 440 bp, Artificial Sequence
  Synthetic
gcagagctcgtttagtgaaccgtcagatcgcctggagacgccatccacgctgttttgacctccatagaagattctagaagcaaaagacggcatacgagatcaccatgcgcagcctgagcgtgctggccctgctgctgctcctgctcctggcccctgcttctgccgctctgaacgacatcttcgaggcccagaagatcgagtggcacgagagcggcggcagcggcactagcagcagaaagaagcgcgctacgtcttcagaattcagaagacacggttcgggaggctcagggtcaggtcgaatgaagcaaatcgaggacaagttggaggagatcttgagcaagttgtaccacatcgagaacgaactagcgcgaatcaagaagttgttgggcgagcgaggatcctgagtgtcggtggtcgccgtatcatcttcgaatgtcgac

SEQ ID NO: 38 - protein, 48 aa, Artificial Sequence
  Synthetic
MRSLSVLALLLLLLLAPASAALNDIFEAQKIEWHESGGSGTSSRKKRA

SEQ ID NO: 39 - DNA, 686 bp, Artificial Sequence
  Synthetic
gcagagctcgtttagtgaaccgtcagatcgcctggagacgccatccacgctgttttgacctccatagaagattctagaagcaaaagacggcatacgagatcaccatgcgcagcctgagcgtgctggccctgctgctgctcctgctcctggcccctgcttctgccgcttccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcaggcccctgaagatttggacatggaggataacgatattattgaggctcacagagaacagattggcggcagcggcactagcagcagaaagaagcgcgctacgtcttcagaattcagaagacacggttcgggaggctcagggtcaggtcgaatgaagcaaatcgaggacaagttggaggagatcttgagcaagttgtaccacatcgagaacgaactagcgcgaatcaagaagttgttgggcgagcgaggatcctgagtgtcggtggtcgccgtatcatcttcgaatgtcgac

SEQ ID NO: 40 - protein, 130 aa, Artificial Sequence
  Synthetic
MRSLSVLALLLLLLLAPASAASLQDSEVNQEAKPEVKPEVKPETHINLKVSDGSSEIFFKIKKTTPLRRLMEAFAKRQGKEMDSLTFLYDGIEIQADQAPEDLDMEDNDIIEAHREQIGGSGTSSRKKRA

SEQ ID NO: 41 - DNA, 611 bp, Artificial Sequence
  Synthetic
gcagagctcgtttagtgaaccgtcagatcgcctggagacgccatccacgctgttttgacctccatagaagattctagaagcaaaagacggcatacgagatcaccatgcgcagcctgagcgtgctggccctgctgctgctcctgctcctggcccctgcttctgccgctacgtcttcagaattcagaagacacggttcgggaggctcagggtcaggtcgaatgaagcaaatcgaggacaagttggaggagatcttgagcaagttgtaccacatcgagaacgaactagcgcgaatcaagaagttgttgggcgagcgaggatcgggtggcgagaacctttacttccaaggtcgcggtggttccgagaacctttacttccaaggtgaaggcggtagcgatgacgacgacaagggcgggggttcggcggtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatcctgagtgtcggtggtcgccgtatcatcttcgaatgtcgac

SEQ ID NO: 42 - protein, 127 aa, Artificial Sequence
  Synthetic
GSGGSGSGRMKQIEDKLEEILSKLYHIENELARIKKLLGERGSGGENLYFQGRGGSENLYFQGEGGSDDDDKGGGSAVGQDTQEVIVVPHSLPFKVVVISAILALVVLTIISLIILIMLWQKKPRGS

SEQ ID NO: 43 - DNA, 1193 bp, Artificial Sequence
  Synthetic
gcagagctcgtttagtgaaccgtcagatcgcctggagacgccatccacgctgttttgacctccatagaagattctagaagcaaaagacggcatacgagatcaccatgcgcagcctgagcgtgctggccctgctgctgctcctgctcctggcccctgcttctgccgctacgtcttcagaattcagaagacacggttcgggaggctcagggtcaggtgataaaactcacacatgcccaccgtgcccagcacctgaactcctggggggaccgtcagtatttctatttccgccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgagggtctgcacaaccactacacgcagaagagcctctccctgtctccgggtaaagggtcgggtggcgagaacctttacttccaaggtcgcggtggttccgagaacctttacttccaaggtgaaggcggtagcgatgacgacgacaagggcgggggttcggcggtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatcctgagtgtcggtggtcgccgtatcatcttcgaatgtcgac

SEQ ID NO: 44 - protein, 321 aa, Artificial Sequence
  Synthetic
GSGGSGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGKGSGGENLYFQGRGGSENLYFQGEGGSDDDDKGGGSAVGQDTQEVIVVPHSLPFKVVVISAILALVVLTIISLIILIMLWQKKPRGS

SEQ ID NO: 45 - protein, 7 aa, Artificial Sequence
  Synthetic
EFLIVKS

SEQ ID NO: 46 - protein, 7 aa, Artificial Sequence
  Synthetic
CXXRGDC

SEQ ID NO: 47 - protein, 12 aa, Artificial Sequence
  Synthetic
HTMYYHHYQHHL

SEQ ID NO: 48 - DNA, 150 bp, Artificial Sequence
  Synthetic
cccaggcacatcaccagcctggaggtgatcaaggccggaccccactgccccactgcccaactcatagccacgctgaagaatgggaggaaaatttgcttggatctgcaagccctgctgtacaagaaaatcattaaggaacatttggagagt

SEQ ID NO: 49 - protein, 50 aa, Artificial Sequence
  Synthetic
PRHITSLEVIKAGPHCPTAQLIATLKNGRKICLDLQALLYKKIIKEHLES

SEQ ID NO: 50 - DNA, 150 bp, Artificial Sequence
  Synthetic
atccagcaggcccggaaagctccttctggacgaatgtccatcgttaagaacctgcagaacctggaccccagccacaggataagtgaccgggactacatgggctggatggattttggccgtcgcagtgccgaggagtatgagtacccctcc

SEQ ID NO: 51 - protein, 50 aa, Artificial Sequence
  Synthetic
IQQARKAPSGRMSIVKNLQNLDPSHRISDRDYMGWMDFGRRSAEEYEYPS

SEQ ID NO: 52 - DNA, 150 bp, Artificial Sequence
  Synthetic
cctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgc

SEQ ID NO: 53 - protein, 50 aa, Artificial Sequence
  Synthetic
PPWTGEVSPAQRDGGALGRGPWDSSDRSALLKSKLRALLTAPRSLRRSSC

SEQ ID NO: 54 - DNA, 150 bp, Artificial Sequence
  Synthetic
atgaagaaccatttgcttttctggggagtcctggcggtttttattaaggctgttcatgtgaaagcccaagaagatgaaaggattgttcttgttgacaacaaatgtaagtgtgcccggattacttccaggatcatccgttcttccgaagat

SEQ ID NO: 55 - protein, 50 aa, Artificial Sequence
  Synthetic
MKNHLLFWGVLAVFIKAVHVKAQEDERIVLVDNKCKCARITSRIIRSSED

SEQ ID NO: 56 - DNA, 150 bp, Artificial Sequence
  Synthetic
gatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgt

SEQ ID NO: 57 - protein, 50 aa, Artificial Sequence
  Synthetic
DVRFESIRLPGCPRGVNPVVSYAVALSCQCALCRRSTTDCGGPKDHPLTC

SEQ ID NO: 58 - DNA, 150 bp, Artificial Sequence
  Synthetic
gtgctgctcggacactctctgggcatcccctgggctcccctgagcagctgccccagccaggccctgcagctggcaggctgcttgagccaactccatagcggccttttcctctaccaggggctcctgcaggccctggaagggatctccccc

SEQ ID NO: 59 - protein, 50 aa, Artificial Sequence
  Synthetic
VLLGHSLGIPWAPLSSCPSQALQLAGCLSQLHSGLFLYQGLLQALEGISP

SEQ ID NO: 60 - DNA, 150 bp, Artificial Sequence
  Synthetic
gaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcag

SEQ ID NO: 61 - protein, 50 aa, Artificial Sequence
  Synthetic
EVVVPLTVELMGTVAKQLVPSCVTVQRCGGCCPDDGLECVPTGQHQVRMQ

SEQ ID NO: 62 - DNA, 150 bp, Artificial Sequence
  Synthetic
aacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacg

SEQ ID NO: 63 - protein, 50 aa, Artificial Sequence
  Synthetic
NKFAKLIVETDTFGSRVRIKGAESEKYICMNKRGKLIGKPSGKSKDCVFT

SEQ ID NO: 64 - DNA, 150 bp, Artificial Sequence
  Synthetic
aagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaac

SEQ ID NO: 65 - protein, 50 aa, Artificial Sequence
  Synthetic
KGYTVGAEASIILGQEQDSFGGNFEGSQSLVGDIGNVNMWDFVLSPDEIN

SEQ ID NO: 66 - DNA, 150 bp, Artificial Sequence
  Synthetic
attgctgccgtgatatttggcttcttggcgactgcggcatatgcagtgaacacattcctggcagtgcagaaatggagagtcagcgtccgccagcagagcaccaatgactacatccgagcccgcacggagtccagggatgtggacagtcgc

SEQ ID NO: 67 - protein, 50 aa, Artificial Sequence
  Synthetic
IAAVIFGFLATAAYAVNTFLAVQKWRVSVRQQSTNDYIRARTESRDVDSR

SEQ ID NO: 68 - DNA, 150 bp, Artificial Sequence
  Synthetic
caacaaacagagctgcagagcctcaggagagaggtgagccggctgcaggggacaggaggcccctcccagaatggggaagggtatccctggcagagtctcccggagcagagttccgatgccctggaagcctgggagaatggggagagatcc

SEQ ID NO: 69 - protein, 50 aa, Artificial Sequence
  Synthetic
QQTELQSLRREVSRLQGTGGPSQNGEGYPWQSLPEQSSDALEAWENGERS

SEQ ID NO: 70 - DNA, 150 bp, Artificial Sequence
  Synthetic
agcatgagcgagaatggctacgacccccagcagaacctgaacgacctgatgctgcttcagctggaccgtgaggccaacctcaccagcagcgtgacgatactgccactgcctctgcagaacgccacggtggaagccggcaccagatgccag

SEQ ID NO: 71 - protein, 50 aa, Artificial Sequence
  Synthetic
SMSENGYDPQQNLNDLMLLQLDREANLTSSVTILPLPLQNATVEAGTRCQ

SEQ ID NO: 72 - DNA, 150 bp, Artificial Sequence
  Synthetic
gggggccccctggtgtgtgggggagtccttcaaggtctggtgtcctgggggtctgtggggccctgtggacaagatggcatccctggagtctacacctatatttgcaactccactcttgttggcctgggaacttcttggaactttaactcc

SEQ ID NO: 73 - protein, 50 aa, Artificial Sequence
  Synthetic
GGPLVCGGVLQGLVSWGSVGPCGQDGIPGVYTYICNSTLVGLGTSWNFNS

SEQ ID NO: 74 - DNA, 150 bp, Artificial Sequence
  Synthetic
cttcccaacgagacaccctgctacatcaccggctggggccgtctctataccaacgggccactcccagacaagctgcaggaggccctgctgccggtggtggactatgaacactgctccaggtggaactggtggggttcctccgtgaaaaag

SEQ ID NO: 75 - protein, 50 aa, Artificial Sequence
  Synthetic
LPNETPCYITGWGRLYTNGPLPDKLQEALLPVVDYEHCSRWNWWGSSVKK

SEQ ID NO: 76 - DNA, 150 bp, Artificial Sequence
  Synthetic
tggaactggtggggttcctccgtgaaaaagaccatggtgtgtgctggaggggacatccgctccggctgcaatggtgactctggaggacccctcaactgccccacagaggatggtggctggcaggtccatggcgtgaccagctttgtttct

SEQ ID NO: 77 - protein, 50 aa, Artificial Sequence
  Synthetic
WNWWGSSVKKTMVCAGGDIRSGCNGDSGGPLNCPTEDGGWQVHGVTSFVS

SEQ ID NO: 78 - DNA, 150 bp, Artificial Sequence
  Synthetic
gtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggc

SEQ ID NO: 79 - protein, 50 aa, Artificial Sequence
  Synthetic
VEETVAEVTEVSVGANPVQVEVGEFDDGAEETEEEVVAENPCQNHHCKHG

SEQ ID NO: 80 - DNA, 150 bp, Artificial Sequence
  Synthetic
caggtcctcatccagcatcttcgagggctccagaaaggcagaagcacagagaggaacgtgtcagtggaagccctggcctctgctctgcagctgttagccagggagcagcaaagcacaggaagggtcgggcgctccctcccgacagaggac

SEQ ID NO: 81 - protein, 50 aa, Artificial Sequence; identical to SEQ ID NO: 22
  Synthetic

SEQ ID NO: 82 - DNA, 150 bp, Artificial Sequence
  Synthetic
cagaaaggcagaagcacagagaggaacgtgtcagtggaagccctggcctctgctctgcagctgttagccagggagcagcaaagcacaggaagggtcgggcgctccctcccgacagaggactgtgagaatgagaaggagcaagctgtgcac

SEQ ID NO: 83 - protein, 50 aa, Artificial Sequence
  Synthetic
QKGRSTERNVSVEALASALQLLAREQQSTGRVGRSLPTEDCENEKEQAVH

SEQ ID NO: 84 - DNA, 150 bp, Artificial Sequence
  Synthetic
tcagtggaagccctggcctctgctctgcagctgttagccagggagcagcaaagcacaggaagggtcgggcgctccctcccgacagaggactgtgagaatgagaaggagcaagctgtgcacaatgtagtccagctgctgccaggagtggga

SEQ ID NO: 85 - protein, 50 aa, Artificial Sequence
  Synthetic
SVEALASALQLLAREQQSTGRVGRSLPTEDCENEKEQAVHNVVQLLPGVG

SEQ ID NO: 86 - DNA, 150 bp, Artificial Sequence
  Synthetic
ctgttagccagggagcagcaaagcacaggaagggtcgggcgctccctcccgacagaggactgtgagaatgagaaggagcaagctgtgcacaatgtagtccagctgctgccaggagtgggaaccttctacaacctgggcacagctttgtat

SEQ ID NO: 87 - protein, 50 aa, Artificial Sequence; identical to SEQ ID NO: 24
  Synthetic

SEQ ID NO: 88 - DNA, 150 bp, Artificial Sequence
  Synthetic
gacatcatcaaacctgacccacccaagaacttgcagctgaagccattaaagaattctcggcaggtggaggtcagctgggagtaccctgacacctggagtactccacattcctacttctccctgacattctgcgttcaggtccagggcaag

SEQ ID NO: 89 - protein, 50 aa, Artificial Sequence
  Synthetic
DIIKPDPPKNLQLKPLKNSRQVEVSWEYPDTWSTPHSYFSLTFCVQVQGK

SEQ ID NO: 90 - DNA, 150 bp, Artificial Sequence
  Synthetic
atcagcttgtctgtttcattccctgatgttacgagcaatatgaccatcttctgtattctggaaactgacaagacgcggcttttatcttcacctttctctatagagcttgaggaccctcagcctcccccagaccacattccttggattaca

SEQ ID NO: 91 - protein, 50 aa, Artificial Sequence
  Synthetic
ISLSVSFPDVTSNMTIFCILETDKTRLLSSPFSIELEDPQPPPDHIPWIT

SEQ ID NO: 92 - DNA, 150 bp, Artificial Sequence
  Synthetic
ttcctttacctgtcagacaacctgctggattctatcccggggcctttgcccctgagcctgcgctctgtacacctgcagaataacctgatagagaccatgcagagagacgtattctgtgaccccgaggagcacaaacacacccgcaggcag

SEQ ID NO: 93 - protein, 50 aa, Artificial Sequence
  Synthetic
FLYLSDNLLDSIPGPLPLSLRSVHLQNNLIETMQRDVFCDPEEHKHTRRQ

SEQ ID NO: 94 - DNA, 150 bp, Artificial Sequence
  Synthetic
gccgtgctgcgcttcttcctctgtgccatgtacgcgcccatttgcaccctggagttcctgcacgaccctatcaagccgtgcaagtcggtgtgccaacgcgcgcgcgacgactgcgagcccctcatgaagatgtacaaccacagctggccc

SEQ ID NO: 95 - protein, 50 aa, Artificial Sequence
  Synthetic
AVLRFFLCAMYAPICTLEFLHDPIKPCKSVCQRARDDCEPLMKMYNHSWP

SEQ ID NO: 96 - DNA, 150 bp, Artificial Sequence
  Synthetic
gatacattggctcagtgtgagcaagaagaagtttatgattgttcacatgatgaagatgctggggcatcgtgtgagaacccagagagctctttctccccagtcccagagggtgtcaggctggctgacggccctgggcattgcaagggacgc

SEQ ID NO: 97 - protein, 50 aa, Artificial Sequence
  Synthetic
DTLAQCEQEEVYDCSHDEDAGASCENPESSFSPVPEGVRLADGPGHCKGR

SEQ ID NO: 98 - DNA, 150 bp, Artificial Sequence
  Synthetic
ctacacaacagtgaagtggggagacaggctctgcgcgcctctctggaaatgaagtgtaagtgccatggggtgtctggctcctgctccatccgcacctgctggaaggggctgcaggagctgcaggatgtggctgctgacctcaagacccga

SEQ ID NO: 99 - protein, 50 aa, Artificial Sequence
  Synthetic
LHNSEVGRQALRASLEMKCKCHGVSGSCSIRTCWKGLQELQDVAADLKTR

SEQ ID NO: 100 - DNA, 150 bp, Artificial Sequence
  Synthetic
tacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtc

SEQ ID NO: 101 - protein, 50 aa, Artificial Sequence
  Synthetic
YTGANKYDEAASYIQSKFEDLNKRKDTKEIYTHFTCATDTKNVQFVFDAV

SEQ ID NO: 102 - DNA, 150 bp, Artificial Sequence
  Synthetic
aagaagccaacgaaaaatgacctcgtgtattttgagaattctccagactactgtatcagggaccgagaggcaggctccctgggtacagcaggccgtgtgtgcaacctgacttcccggggcatggacagctgtgaagtcatgtgctgtggg

SEQ ID NO: 103 - protein, 50 aa, Artificial Sequence
  Synthetic
KKPTKNDLVYFENSPDYCIRDREAGSLGTAGRVCNLTSRGMDSCEVMCCG

SEQ ID NO: 104 - DNA, 150 bp, Artificial Sequence
  Synthetic
ctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggag

SEQ ID NO: 105 - protein, 50 aa, Artificial Sequence
  Synthetic
LMLCAATAVLLSAQGGPVQSKSPRFASWDEMNVLAHGLLQLGQGLREHAE

SEQ ID NO: 106 - DNA, 150 bp, Artificial Sequence
  Synthetic
gcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacg

SEQ ID NO: 107 - protein, 50 aa, Artificial Sequence
  Synthetic
AGEAEPSGAARSASSGREEPQPEEGEEEEEKEEERGPQWRLGARKPGSWT

SEQ ID NO: 108 - DNA, 150 bp, Artificial Sequence
  Synthetic
ctgggtgtgcccctcatccgcagcggcctcttccactcccacctggagaacctgcagcaggtgcccacctcggagctccacgagcaggtgacgctgagctacggtatgtttgaaaacaagcggaacgccgtccacgtgaaggggcccttc

SEQ ID NO: 109 - protein, 50 aa, Artificial Sequence
  Synthetic
LGVPLIRSGLFHSHLENLQQVPTSELHEQVTLSYGMFENKRNAVHVKGPF

SEQ ID NO: 110 - DNA, 150 bp, Artificial Sequence
  Synthetic
agttcctgggcagaaactacttattggatatcaccacaaggaattccagaaactaaagttcaggatatggattgcgtatattacaattggcaatatttactctgttcttggaaacctggcataggtgtacttcttgataccaattacaac

SEQ ID NO: 111 - protein, 50 aa, Artificial Sequence
  Synthetic
SSWAETTYWISPQGIPETKVQDMDCVYYNWQYLLCSWKPGIGVLLDTNYN

SEQ ID NO: 112 - DNA, 150 bp, Artificial Sequence
  Synthetic
ctccagctcttggaggcagcagtggtcaaagtgcccctgaagaaatttaagtctatccgtgagaccatgaaggagaagggcttgctgggggagttcctgaggacccacaagtatgatcctgcttggaagtaccgctttggtgacctcagc

SEQ ID NO: 113 - protein, 50 aa, Artificial Sequence
  Synthetic
LQLLEAAVVKVPLKKFKSIRETMKEKGLLGEFLRTHKYDPAWKYRFGDLS

SEQ ID NO: 114 - DNA, 150 bp, Artificial Sequence
  Synthetic
tcaaaacatagcgggcctgaaaataaccagtgttccctccaccctttccaaatcagcttccgccagctgggttgggatcactggatcattgctccccctttctacaccccaaactactgtaaaggaacttgtctccgagtactacgcgat

SEQ ID NO: 115 - protein, 50 aa, Artificial Sequence
  Synthetic
SKHSGPENNQCSLHPFQISFRQLGWDHWIIAPPFYTPNYCKGTCLRVLRD

SEQ ID NO: 116 - DNA, 150 bp, Artificial Sequence
  Synthetic
gtcacctccctggggccgggagccgaggggctgcatccattcatggagcttcgagtcctagagaacacaaaacgttcccggcggaacctgggtctggactgcgacgagcactcaagcgagtcccgctgctgccgatatcccctcacagtg

SEQ ID NO: 117 - protein, 50 aa, Artificial Sequence
  Synthetic
VTSLGPGAEGLHPFMELRVLENTKRSRRNLGLDCDEHSSESRCCRYPLTV

SEQ ID NO: 118 - DNA, 150 bp, Artificial Sequence
  Synthetic
cacacggctgtggtgaaccagtaccgcatgcggggtctgaaccccggcacggtgaactcctgctgcattcccaccaagctgagcaccatgtccatgctgtacttcgatgatgagtacaacatcgtcaagcgggacgtgcccaacatgatt

SEQ ID NO: 119 - protein, 50 aa, Artificial Sequence
  Synthetic
HTAVVNQYRMRGLNPGTVNSCCIPTKLSTMSMLYFDDEYNIVKRDVPNMI

SEQ ID NO: 120 - DNA, 150 bp, Artificial Sequence
  Synthetic
atcttcagcttgctgggtctgcttggagagatcacctacattgtgctgctggtgcttcatactgtctggaacggcaatggcatgattggcttccaggtcctcctcagcattggggaactcagcttggccatcgtgatagctctcacgtct

SEQ ID NO: 121 - protein, 50 aa, Artificial Sequence
  Synthetic
IFSLLGLLGEITYIVLLVLHTVWNGNGMIGFQVLLSIGELSLAIVIALTS

SEQ ID NO: 122 - DNA, 150 bp, Artificial Sequence
  Synthetic
ctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcagga

SEQ ID NO: 123 - protein, 50 aa, Artificial Sequence
  Synthetic
LDQGKSSLDVRIACEQCQESGASLVLLGKKKKKEEEGEGKKKGGGEGGAG

SEQ ID NO: 124 - DNA, 150 bp, Artificial Sequence
  Synthetic
gagagaattgttgatgttgctggaccagggggttggaatgacccagatatgttagtgattggcaactttggcctcagctggaatcagcaagtaactcagatggccctctgggctatcatggctgctcctttattcatgtctaatgacctc

SEQ ID NO: 125 - protein, 50 aa, Artificial Sequence
  Synthetic
ERIVDVAGPGGWNDPDMLVIGNFGLSWNQQVTQMALWAIMAAPLFMSNDL

SEQ ID NO: 126 - DNA, 150 bp, Artificial Sequence
  Synthetic
gccccatgcgagcagcgctgcttcaactcctatgggaccttcctgtgtcgctgccaccagggctatgagctgcatcgggatggcttctcctgcagtgatattgatgagtgtagctactccagctacctctgtcagtaccgctgcatcaac

SEQ ID NO: 127 - protein, 50 aa, Artificial Sequence
  Synthetic
APCEQRCFNSYGTFLCRCHQGYELHRDGFSCSDIDECSYSSYLCQYRCIN

SEQ ID NO: 128 - DNA, 150 bp, Artificial Sequence
  Synthetic
tgcagtgatattgatgagtgtagctactccagctacctctgtcagtaccgctgcatcaacgagccaggccgtttctcctgccactgcccacagggttaccagctgctggccacacgcctctgccaagacattgatgagtgtgagtctggt

SEQ ID NO: 129 - protein, 50 aa, Artificial Sequence
  Synthetic
CSDIDECSYSSYLCQYRCINEPGRFSCHCPQGYQLLATRLCQDIDECESG

SEQ ID NO: 130 - DNA, 150 bp, Artificial Sequence
  Synthetic
cagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcg

SEQ ID NO: 131 - protein, 50 aa, Artificial Sequence
  Synthetic
QNGRCLREAQYSMLATWRRRTPRREATLELLGRVLRDMDLLGCLEDIEEA

SEQ ID NO: 132 - DNA, 150 bp, Artificial Sequence
  Synthetic
ttgggccgcgagctgatgctgcagctgtcggagtttctgtgcgaggagttccggaacaggaaccagcgcatcgtccagctcatccaggacacgcgcattcacatcctgccatccatgaaccccgacggctacgaggtggctgctgcccag

SEQ ID NO: 133 - protein, 50 aa, Artificial Sequence
  Synthetic
LGRELMLQLSEFLCEEFRNRNQRIVQLIQDTRIHILPSMNPDGYEVAAAQ

SEQ ID NO: 134 - DNA, 150 bp, Artificial Sequence
  Synthetic
ttccagaagctggccaaggtctactcctatgcacatggatggatgttccaaggttggaactgcggagattacttcccagatggcatcaccaatggggcttcctggtattctctcagcaagggaatgcaagactttaattatctccatacc

SEQ ID NO: 135 - protein, 50 aa, Artificial Sequence
  Synthetic
FQKLAKVYSYAHGWMFQGWNCGDYFPDGITNGASWYSLSKGMQDFNYLHT

SEQ ID NO: 136 - DNA, 150 bp, Artificial Sequence
  Synthetic
agcctgggagcccacgtggctggagaggcaggaagcaagactccaggcctgagcaggattacagggttggatcctgtagaagcaagtttcgagagtactcctgaagaggtgcgacttgatccctctgatgctgactttgttgatgtgatt

SEQ ID NO: 137 - protein, 50 aa, Artificial Sequence
  Synthetic
SLGAHVAGEAGSKTPGLSRITGLDPVEASFESTPEEVRLDPSDADFVDVI

SEQ ID NO: 138 - DNA, 150 bp, Artificial Sequence
  Synthetic
ggaagcaagactccaggcctgagcaggattacagggttggatcctgtagaagcaagtttcgagagtactcctgaagaggtgcgacttgatccctctgatgctgactttgttgatgtgattcacacggatgcagctcccctgatcccattc

SEQ ID NO: 139 - protein, 50 aa, Artificial Sequence
  Synthetic
GSKTPGLSRITGLDPVEASFESTPEEVRLDPSDADFVDVIHTDAAPLIPF

SEQ ID NO: 140 - DNA, 150 bp, Artificial Sequence
  Synthetic
aaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattct

SEQ ID NO: 141 - protein, 50 aa, Artificial Sequence
  Synthetic
KFPSGTFEQVSQLVKEVVSLTEACCAEGADPDCYDTRTSALSAKSCESNS

SEQ ID NO: 142 - DNA, 150 bp, Artificial Sequence
  Synthetic
tactacaagaggctgggccgcgacgcgctgctcagctgggacgacgtgctggccgtgcagagcctgtatgggaagcccctagggggctcagtggccgtccagctcccaggaaagctgttcactgactttgagacctgggactcctacagc

SEQ ID NO: 143 - protein, 50 aa, Artificial Sequence
  Synthetic
YYKRLGRDALLSWDDVLAVQSLYGKPLGGSVAVQLPGKLFTDFETWDSYS

SEQ ID NO: 144 - DNA, 150 bp, Artificial Sequence
  Synthetic
atgcggctgcggctccggcttctggcgctgctgcttctgctgctggcaccgcccgcgcgcgccccgaagccctcggcgcaggacgtgagcctgggcgtggactggctgactcgctatggttacctgccgccaccccaccctgcccaggcc

SEQ ID NO: 145 - protein, 50 aa, Artificial Sequence
  Synthetic
MRLRLRLLALLLLLLAPPARAPKPSAQDVSLGVDWLTRYGYLPPPHPAQA

SEQ ID NO: 146 - DNA, 150 bp, Artificial Sequence
  Synthetic
tctgggacgtactgtgtgaacctcaccctgggggatgacacaagcctggctctcacgagcaccctgatttctgttcctgacagagacccagcctcgcctttaaggatggcaaacagtgccctgatctccgttggctgcttggccatattt

SEQ ID NO: 147 - protein, 50 aa, Artificial Sequence
  Synthetic
SGTYCVNLTLGDDTSLALTSTLISVPDRDPASPLRMANSALISVGCLAIF

SEQ ID NO: 148 - DNA, 150 bp, Artificial Sequence
  Synthetic
aacgcgctcctgttcgcggaggaggaggacggggaagccggcgccgaggacaagcgctcccaggaggagacgccgggccaccggcggaaggaggccgaggggacagaggagggcggggaggaggaggacgacgaggagatggatccgcag

SEQ ID NO: 149 - protein, 50 aa, Artificial Sequence
  Synthetic
NALLFAEEEDGEAGAEDKRSQEETPGHRRKEAEGTEEGGEEEDDEEMDPQ

SEQ ID NO: 150 - DNA, 150 bp, Artificial Sequence
  Synthetic
ctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggat

SEQ ID NO: 151 - protein, 50 aa, Artificial Sequence
  Synthetic
LLLGPAGARAQEDEDGDYEELVLALRSEEDGLAEAPEHGTTATFHRCAKD

SEQ ID NO: 152 - DNA, 150 bp, Artificial Sequence
  Synthetic
cactcttacctgtgcacagcaacttgtgaatctaggaaattggaaaaaggaatccaggtggagatctactcttttcctaaggatccagagattcatttgagtggccctctggaggctgggaagccgatcacagtcaagtgttcagttgct

SEQ ID NO: 153 - protein, 50 aa, Artificial Sequence
  Synthetic
HSYLCTATCESRKLEKGIQVEIYSFPKDPEIHLSGPLEAGKPITVKCSVA

SEQ ID NO: 154 - DNA, 150 bp, Artificial Sequence
  Synthetic
aacagtgactgtacgcacgatgaggatgctggggtcatctgcaaagaccagcgcctccctggcttctcggactccaatgtcattgaggtagagcatcacctgcaagtggaggaggtgcgaattcgacccgccgttgggtggggcagacga

SEQ ID NO: 155 - protein, 50 aa, Artificial Sequence
  Synthetic
NSDCTHDEDAGVICKDQRLPGFSDSNVIEVEHHLQVEEVRIRPAVGWGRR

SEQ ID NO: 156 - DNA, 150 bp, Artificial Sequence
  Synthetic
gacagcgatcaagaccaggatggagacggacatcaggactctcgggacaactgtcccacggtgcctaacagtgcccaggaggactcagaccacgatggccagggtgatgcctgcgacgacgacgacgacaatgacggagtccctgacagt

SEQ ID NO: 157 - protein, 50 aa, Artificial Sequence
  Synthetic
DSDQDQDGDGHQDSRDNCPTVPNSAQEDSDHDGQGDACDDDDDNDGVPDS

SEQ ID NO: 158 - DNA, 150 bp, Artificial Sequence
  Synthetic
catcaggactctcgggacaactgtcccacggtgcctaacagtgcccaggaggactcagaccacgatggccagggtgatgcctgcgacgacgacgacgacaatgacggagtccctgacagtcgggacaactgccgcctggtgcctaacccc

SEQ ID NO: 159 - protein, 50 aa, Artificial Sequence
  Synthetic
HQDSRDNCPTVPNSAQEDSDHDGQGDACDDDDDNDGVPDSRDNCRLVPNP

SEQ ID NO: 160 - DNA, 150 bp, Artificial Sequence
  Synthetic
ggaagagtcccctatccacggccaggaacttgtcccagcaaaacatttggtggttttgactctacaaaggaccttcctgatgatgttataacctttgcaagaagtcatccagccatgtacaatccagtgtttcctatgaacaatcgccca

SEQ ID NO: 161 - protein, 50 aa, Artificial Sequence
  Synthetic
GRVPYPRPGTCPSKTFGGFDSTKDLPDDVITFARSHPAMYNPVFPMNNRP

SEQ ID NO: 162 - DNA, 150 bp, Artificial Sequence
  Synthetic
ggctacacagggcacggcattgtggtctccattctggacgatggcatcgagaagaaccacccggacttggcaggcaattatgatcctggggccagttttgatgtcaatgaccaggaccctgacccccagcctcggtacacacagatgaat

SEQ ID NO: 163 - protein, 50 aa, Artificial Sequence
  Synthetic
GYTGHGIVVSILDDGIEKNHPDLAGNYDPGASFDVNDQDPDPQPRYTQMN

SEQ ID NO: 164 - DNA, 150 bp, Artificial Sequence
  Synthetic
aatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccag

SEQ ID NO: 165 - protein, 50 aa, Artificial Sequence
  Synthetic
NDVETIRASVCAPCHASCATCQGPALTDCLSCPSHASLDPVEQTCSRQSQ

SEQ ID NO: 166 - DNA, 150 bp, Artificial Sequence
  Synthetic
aatgaaattttggggcctgttattcaatttcttggggttccatatgcagccccaccaacaggggaacgtcgttttcagcctccagaaccaccatctccctggtcagatatcagaaatgccactcaatttgctcctgtgtgtccccagaat

SEQ ID NO: 167 - protein, 50 aa, Artificial Sequence
  Synthetic
NEILGPVIQFLGVPYAAPPTGERRFQPPEPPSPWSDIRNATQFAPVCPQN

SEQ ID NO: 168 - DNA, 150 bp, Artificial Sequence
  Synthetic
gtggcctggtccaaatacaatccccgagaccagctctaccttcacatcgggctgaaaccaagggtccgagatcattaccgggccactaaggtggccttttggaaacatctggtgccccacctatacaacctgcatgacatgttccactat

SEQ ID NO: 169 - protein, 50 aa, Artificial Sequence
  Synthetic
VAWSKYNPRDQLYLHIGLKPRVRDHYRATKVAFWKHLVPHLYNLHDMFHY

SEQ ID NO: 170 - DNA, 150 bp, Artificial Sequence
  Synthetic
aagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagcc

SEQ ID NO: 171 - protein, 50 aa, Artificial Sequence
  Synthetic
KNWYKKSICGQKTTVLYECCPGYMRMEGMKGCPAVLPIDHVYGTLGIVGA

SEQ ID NO: 172 - DNA, 150 bp, Artificial Sequence
  Synthetic
ctggctgaggatgggaagaggtgtgtggctgtggactactgtgcctcagaaaaccacggatgtgaacatgagtgtgtaaatgctgatggctcctacctttgccagtgccatgaaggatttgctcttaacccagataaaaaaacgtgcaca

SEQ ID NO: 173 - protein, 50 aa, Artificial Sequence
  Synthetic
LAEDGKRCVAVDYCASENHGCEHECVNADGSYLCQCHEGFALNPDKKTCT

SEQ ID NO: 174 - DNA, 150 bp, Artificial Sequence
  Synthetic
aagatggagcctcaggaggtggagtccctgggggagacctatgacttcgacagcatcatgcattacgctcggaacacattctccaggggcatcttcctggataccattgtccccaagtatgaggtgaacggggtgaaacctcccattggc

SEQ ID NO: 175 - protein, 50 aa, Artificial Sequence
  Synthetic
KMEPQEVESLGETYDFDSIMHYARNTFSRGIFLDTIVPKYEVNGVKPPIG

SEQ ID NO: 176 - DNA, 150 bp, Artificial Sequence
  Synthetic
gcgaaaatcgacgacaaaggcgttgtaaccaagggtgctgacgttactgacgttaaagatccactggctaccctggacaaagcgctggcacaggttgacggcctgcgttcttccctgggtgcggtacagaaccgtttcgattctgttatc

SEQ ID NO: 177 - protein, 50 aa, Artificial Sequence
  Synthetic
AKIDDKGVVTKGADVTDVKDPLATLDKALAQVDGLRSSLGAVQNRFDSVI

SEQ ID NO: 178 - DNA, 255 bp, Artificial Sequence
  Synthetic
atgcgcagcctgagcgtgctggccctgctgctgctcctgctcctggcccctgcttctgccgcttggagtcatccccagttcgagaaaggcggcggcactggcggcggctcaggtggtggttcgggttcgggaggctcagggtcaggtcgaatgaagcaaatcgaggacaagttggaggagatcttgagcaagttgtaccacatcgagaacgaactagcgcgaatcaagaagttgttgggcgagcgaggatcctga

SEQ ID NO: 179 - protein, 84 aa, Artificial Sequence
  Synthetic
MRSLSVLALLLLLLLAPASAAWSHPQFEKGGGTGGGSGGGSGSGGSGSGRMKQIEDKLEEILSKLYHIENELARIKKLLGERGS

SEQ ID NO: 180 - DNA, 201 bp, Artificial Sequence
  Synthetic
atgggcgcttggagtcatccccagttcgagaaaggcggcggcactggcggcggctcaggtggtggttcgggttcgggaggctcagggtcaggtcgaatgaagcaaatcgaggacaagttggaggagatcttgagcaagttgtaccacatcgagaacgaactagcgcgaatcaagaagttgttgggcgagcgaggatcctga

SEQ ID NO: 181 - protein, 65 aa, Artificial Sequence
  Synthetic
MGAWSHPQFEKGGGTGGGSGGSGSGGSGSGRMKQIEDKLEEILSKLYHIENELARIKKLLGERGS

SEQ ID NO: 182 - protein, 84 aa, Artificial Sequence
  Synthetic
MRSLSVLALLLLLLLAPASAADYKDDDDKGGGTGGGSGGGSGSGGSGSGRMKQIEDKLEEILSKLYHIENELARIKKLLGERGS

SEQ ID NO: 183 - DNA, 335 bp, Artificial Sequence
  Synthetic
atgcgcagcctgagcgtgctggccctgctgctgctcctgctcctggcccctgcttctgccgctctgaacgacatcttcgaggcccagaagatcgagtggcacgagagcggcggcagcggcactagcagcagaaagaagcgcgcttggagtcatccccagttcgagaaaggcggcggcactggcggcggctcaggtggtggttcgggttcgggaggctcagggtcaggtcgaatgaagcaatcgaggacaagttggaggagatcttgagcaagttgtaccacatcgagaacgaactagcgcgaatcaagaagttgttgggcgagcgaggatcctga

SEQ ID NO: 184 - DNA, 336 bp, Artificial Sequence
  Synthetic
atgcgcagcctgagcgtgctggccctgctgctgctcctgctcctggcccctgcttctgcggcgctgaacgacatcttcgaggcccagaagatcgagtggcacgagagcggcggcagcggcactagcagcagaaagaagagagcatggagtcatccccagttcgagaaaggcggcggcactggcggcggctcaggtggtggttcgggttcgggaggctcagggtcaggtcgaatgaagcaaatcgaggacaagttggaggagatcttgagcaagttgtaccacatcgagaacgaactagcgcgaatcaagaagttgttgggcgagcgagggtcgtga

SEQ ID NO: 185 - protein, 111 aa, Artificial Sequence
  Synthetic
MRSLSVLALLLLLLLAPASAALNDIFEAQKIEWHESGGSGTSSRKKRAWSHPQFEKGGGTGGGSGGGSGSGGSGSGRMKQIEDKLEEILSKLYHIENELARIKKLLGERGS

SEQ ID NO: 186 - DNA, 582 bp, Artificial Sequence
  Synthetic
atgcgcagcctgagcgtgctggccctgctgctgctcctgctcctggcccctgcttctgccgcttccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcaggcccctgaagatttggacatggaggataacgatattattgaggctcacagagaacagattggcggcagcggcactagcagcagaaagaagcgcgcttggagtcatccccagttcgagaaaggcggcggcactggcggcggctcaggtggtggttcgggttcgggaggctcagggtcaggtcgaatgaagcaaatcgaggacaagttggaggagatcttgagcaagttgtaccacatcgagaacgaactagcgcgaatcaagaagttgttgggcgagcgaggatcctga

SEQ ID NO: 187 - protein, 193 aa, Artificial Sequence
  Synthetic
MRSLSVLALLLLLLLAPASAASLQDSEVNQEAKPEVKPEVKPETHINLKVSDGSSEIFFKIKKTTPLRRLMEAFAKRQGKEMDSLTFLYDGIEIQADQAPEDLDMEDNDIIEAHREQIGGSGTSSRKKRAWSHPQFEKGGGTGGGSGGGSGSGGSGSGRMKQIEDKLEEILSKLYHIENELARIKKLLGERGS

SEQ ID NO: 188 - protein, 204 aa, Artificial Sequence
  Synthetic
MRSLSVLALLLLLLLAPASAASDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSGTSSRKKRAWSHPQFEKGGGTGGGSGGGSGSGGSGSGRMKQIEDKLEEILSKLYHIENELARIKKLLGERGS

SEQ ID NO: 189 - DNA, 507 bp, Artificial Sequence
  Synthetic
atgcgcagcctgagcgtgctggccctgctgctgctcctgctcctggcccctgcttctgccgcttggagtcatccccagttcgagaaaggcggcggcactggcggcggctcaggtggtggttcgggttcgggaggctcagggtcaggtcgaatgaagcaaatcgaggacaagttggaggagatcttgagcaagttgtaccacatcgagaacgaactagcgcgaatcaagaagttgttgggcgagcgaggatcgggtggcgagaacctttacttccaaggtcgcggtggttccgagaacctttacttccaaggtgaaggcggtagcgatgacgacgacaagggcgggggttcggcggtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatcctga

SEQ ID NO: 190 - protein, 168 aa, Artificial Sequence
  Synthetic
MRSLSVLALLLLLLLAPASAAWSHPQFEKGGGTGGGSGGGSGSGGSGSGRMKQIEDKLEEILSKLYHIENELARIKKLLGERGSGGENLYFQGRGGSENLYFQGEGGSDDDDKGGGSAVGQDTQEVIVVPHSLPFKVVVISAILALVVLTIISLIILIMLWQKKPRGS

SEQ ID NO: 191 - DNA, 1089 bp, Artificial Sequence
  Synthetic
atgcgcagcctgagcgtgctggccctgctgctgctcctgctcctggcccctgcttctgccgcttggagtcatccccagttcgagaaaggcggcggcactggcggcggctcaggtggtggttcgggttcgggaggctcagggtcaggtgataaaactcacacatgcccaccgtgcccagcacctgaactcctggggggaccgtcagtatttctatttccgccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgagggtctgcacaaccactacacgcagaagagcctctccctgtctccgggtaaagggtcgggtggcgagaacctttacttccaaggtcgcggtggttccgagaacctttacttccaaggtgaaggcggtagcgatgacgacgacaagggcgggggttcggcggtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatcctga

SEQ ID NO: 192 - protein, 362 aa, Artificial Sequence
  Synthetic
MRSLSVLALLLLLLLAPASAAWSHPQFEKGGGTGGGSGGGSGSGGSGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGKGSGGENLYFQGRGGSENLYFQGEGGSDDDDKGGGSAVGQDTQEVIVVPHSLPFKVVVISAILALVVLTIISLIILIMLWQKKPRGS

SEQ ID NO: 193 - protein, 193 aa, Artificial Sequence
  Synthetic
MRSLSVLALLLLLLLAPASAALNDIFEAQKIEWHESGGSGTSSRKKRAWSHPQFEKGGGTGGGSGGGSGSGGSGSGRMKQIEDKLEEILSKLYHIENELARIKKLLGERGSGGENLYFQGRGGSENLYFQGEGGSDDDDKGGGSAVGQDTQEVIVVPHSLPFKVVVISAILALVVLTIISLIILIMLWQKKPR

SEQ ID NO: 194 - protein, 387 aa, Artificial Sequence
  Synthetic
MRSLSVLALLLLLLLAPASAALNDIFEAQKIEWHESGGSGTSSRKKRAWSHPQFEKGGGTGGGSGGGSGSGGSGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGKGSGGENLYFQGRGGSENLYFQGEGGSDDDDKGGGSAVGQDTQEVIVVPHSLPFKVVVISAILALVVLTIISLIILIMLWQKKPR

SEQ ID NO: 195 - protein, 26 aa, Artificial Sequence
  Synthetic
LNDIFEAQKIEWHESGGSGTSSRKKR

SEQ ID NO: 196 - DNA, 291 bp, Artificial Sequence
  Synthetic
tccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcaggcccctgaagatttggacatggaggataacgatattattgaggctcacagagaacagatt

SEQ ID NO: 197 - protein, 108 aa, Artificial Sequence
  Synthetic
SLQDSEVNQEAKPEVKPEVKPETHINLKVSDGSSEIFFKIKKTTPLRRLMEAFAKRQGKEMDSLTFLYDGIEIQADQAPEDLDMEDNDIIEAHREQIGGSGTSSRKKR

SEQ ID NO: 198 - protein, 119 aa, Artificial Sequence
  Synthetic
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSGTSSRKKR

SEQ ID NO: 199 - protein, 20 aa, Artificial Sequence
  Synthetic
WSHPQFEKGGGTGGGSGGGS

SEQ ID NO: 200 - protein, 20 aa, Artificial Sequence
  Synthetic
DYKDDDDKGGGTGGGSGGGS

SEQ ID NO: 201 - protein, 49 aa, Artificial Sequence
  Synthetic
AVGQDTQEVIVVPHSLPFKVVVISAILALVVLTIISLIILIMLWQKKPR

SEQ ID NO: 202 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 203 - DNA, 681 bp, Artificial Sequence
  Synthetic
gacaaaactcacacatgcccaccgtgcccagcacctgaactcctggggggaccgtcagtgttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgagggtctgcacaaccactacacgcagaagagcctctccctgtctccgggtaaa

SEQ ID NO: 204 - DNA, 681 bp, Artificial Sequence
  Synthetic
gataaaactcacacatgcccaccgtgcccagcacctgaactcctggggggaccgtcagtatttctatttccgccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgagggtctgcacaaccactacacgcagaagagcctctccctgtctccgggtaaa

SEQ ID NO: 205 - DNA, 24 bp, Artificial Sequence
  Synthetic
acctgaccctgagcctcccgaacc

SEQ ID NO: 206 - DNA, 115 bp, Artificial Sequence
  Synthetic
ctagaagcaaaagacggcatacgagatcaccatgcgcagcctgagcgtgctggccctgctgctgctcctgctcctggcccctgcttctgccgctacgtcttcagaattctgtcga

SEQ ID NO: 207 - DNA, 50 bp, Artificial Sequence
  Synthetic
aattctggatcctgagtgtcggtggtcgccgtatcatcttcgaatgtcga

SEQ ID NO: 208 - DNA, 142 bp, Artificial Sequence
  Synthetic
aattcagaagacacggttcgggaggctcagggtcaggtcgaatgaagcaaatcgaggacaagttggaggagatcttgagcaagttgtaccacatcgagaacgaactagcgcgaatcaagaagttgttgggcgagcgaggatc

SEQ ID NO: 209 - DNA, 68 bp, Artificial Sequence
  Synthetic
cgcttggagtcatccccagttcgagaaaggcggcggcactggcggcggctcaggtggtggttcgggtt

SEQ ID NO: 210 - DNA, 113 bp, Artificial Sequence
  Synthetic
cgctctgaacgacatcttcgaggcccagaagatcgagtggcacgagagcggcggcagcggcactagcagcagaaagaagcgcgctacgtcttcagaattcagaagacacggtt

SEQ ID NO: 211 - DNA, 54 bp, Artificial Sequence
  Synthetic
ctagaagcaaaagacggcatacgagatcaccatgggcgctacgtcttcagaatt

SEQ ID NO: 212 - DNA, 373 bp, Artificial Sequence
  Synthetic
cgtctcacgcttccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcaggcccctgaagatttggacatggaggataacgatattattgaggctcacagagaacagattggcggcagcggcactagcagcagaaagaagcgcgctacgtcttcagaattcagaagacacggtttgagacg

SEQ ID NO: 213 - DNA, 296 bp, Artificial Sequence
  Synthetic
cgtctcagatcgggtggcgagaacctttacttccaaggtcgcggtggttccgagaacctttacttccaaggtgaaggcggtagcgatgacgacgacaagggcgggggttcggcggtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatcctgagtgtcggtggtcgccgtatcatcttcgaa

SEQ ID NO: 214 - DNA, 978 bp, Artificial Sequence
  Synthetic
gaattcagaagacacggttcgggaggctcagggtcaggtgataaaactcacacatgcccaccgtgcccagcacctgaactcctggggggaccgtcagtatttctatttccgccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtgttctcatgctccgtgatgcatgagggtctgcacaaccactacacgcagaagagcctctccctgtctccgggtaaagggtcgggtggcgagaacctttacttccaaggtcgcggtggttccgagaacctttacttccaaggtgaaggcggtagcgatgacgacgacaagggcgggggttcggcggtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatcc

SEQ ID NO: 215 - protein, 44 aa, Artificial Sequence
  Synthetic
MLGPCMLLLLLLLGLRLQLSLGIIPVEEENPDFWNREAAEALGA

SEQ ID NO: 216 - protein, 54 aa, Artificial Sequence
  Synthetic
MLLLLLLLGLRLQLSLGGSGGRMKQIEDKIEEILSKIYHIENEIARIKKLIGER

SEQ ID NO: 217 - protein, 84 aa, Artificial Sequence
  Synthetic
MLLLLLLLGLRLQLSLGGSGSDCRTLNLSVVAVSLAVGQDTQEVIVVPHSLPFKVVVISAILALVVLTIISLIILIMLWQKKPR

SEQ ID NO: 218 - protein, 80 aa, Artificial Sequence
  Synthetic
MLLLLLLLGLRLQLSLGGSGGRMKQIEDKIEEILSKIYHIENEIARIKKLIGERGGASRVGRSLPTEDCENEEKEQAVHG

SEQ ID NO: 219 - protein, 110 aa, Artificial Sequence
  Synthetic
MLLLLLLLGLRLQLSLGGSGGRMKQIEDKIEEILSKIYHIENEIARIKKLIGERGGASLLAREQQSTGRVGRSLPTEDCENEEKEQAVHNVVQLLPGVGTFYNLGTALYG

SEQ ID NO: 220 - protein, 79 aa, Artificial Sequence
  Synthetic
MLLLLLLLGLRLQLSLGGSGGRMKQIEDKIEEILSKIYHIENEIARIKKLIGERGGASHQDSRDNCPTVPNSAQEDSDG

SEQ ID NO: 221 - protein, 108 aa, Artificial Sequence
  Synthetic
MLLLLLLLGLRLQLSLGGSGGRMKQIEDKIEEILSKIYHIENEIARIKKLIGERGGASDSDQDQDGDGHQDSRDNCPTVPNSAQEDSDHDGQDACDDDDDNDGVPDSG

SEQ ID NO: 222 - protein, 17 aa, Artificial Sequence
  Synthetic
MLLLLLLLGLRLQLSLG

SEQ ID NO: 223 - DNA, 48 bp, Artificial Sequence
  Synthetic
ctgctgctgctgctgctgctgggcctgaggctacagctctccctgggc

SEQ ID NO: 224 - protein, 22 aa, Artificial Sequence
  Synthetic
MWWRLWWLLLLLLLLWPMVWAA

SEQ ID NO: 225 - DNA, 63 bp, Artificial Sequence
  Synthetic
atgtggtggcgcctgtggtggctgctgctgctgctgctgctgctgtggcccatggtgtgggcc

SEQ ID NO: 226 - protein, 23 aa, Artificial Sequence
  Synthetic
MRPTWAWWLFLVLLLALWAPARG

SEQ ID NO: 227 - DNA, 69 bp, Artificial Sequence
  Synthetic
atgcgccccacctgggcctggtggctgttcctggtgctgctgctggccctgtgggcccccgcccgcggc

SEQ ID NO: 228 - protein, 28 aa, Artificial Sequence
  Synthetic
MAGPLRAPLLLLAILAVALAVSPAAGSS

SEQ ID NO: 229 - protein, 19 aa, Artificial Sequence
  Synthetic
MKLVFLVLLFLGALGLCLA

SEQ ID NO: 230 - DNA, 57 bp, Artificial Sequence
  Synthetic
atgaagctggtgttcctggtgctgctcttcctgggcgctctgggcctgtgcctggcc

SEQ ID NO: 231 - protein, 27 aa, Artificial Sequence
  Synthetic
MGVHECPAWLWLLLSLLSLPLGLPVLG

SEQ ID NO: 232 - protein, 23 aa, Artificial Sequence
  Synthetic
MERMLPLLALGLLAAGFCPAVLC

SEQ ID NO: 233 - protein, 23 aa, Artificial Sequence
  Synthetic
MGRMLPLLALLLLAAGFCPAVLA

SEQ ID NO: 234 - DNA, 69 bp, Artificial Sequence
  Synthetic
atgggcagcatgctgcccctgctggccctgctgctgctggccgctggattctgccccgctgtgctggcc

SEQ ID NO: 235 - protein, 16 aa, Artificial Sequence
  Synthetic
MLGIWTLLPLVLTSVA

SEQ ID NO: 236 - protein, 28 aa, Artificial Sequence
  Synthetic
MNIKGSPWKGSLLLLLVSNLLLCQSVAP

SEQ ID NO: 237 - protein, 16 aa, Artificial Sequence
  Synthetic
MRLAVVCLCLFGLASC

SEQ ID NO: 238 - protein, 21 aa, Artificial Sequence
  Synthetic
MRSLSVLALLLLLLLAPASAA

SEQ ID NO: 239 - DNA, 60 bp, Artificial Sequence
  Synthetic
atgcgcagcctgagcgtgctggccctgctgctgctcctgctcctggcccctgcttctgcc

SEQ ID NO: 240 - protein, 22 aa, Artificial Sequence
  Synthetic
MKSLSALVLLLLLLLLPGALAA

SEQ ID NO: 241 - DNA, 63 bp, Artificial Sequence
  Synthetic
atgaagagcctgagcgccctggtgctgctgctgctcctgctgctcctgcctggagccctggcc

SEQ ID NO: 242 - protein, 25 aa, Artificial Sequence
  Synthetic
MRGAALVLLLLLLLLLALALAAPVP

SEQ ID NO: 243 - protein, 23 aa, Artificial Sequence
  Synthetic
MRGAALVLLLLLLLLLAGVLAAP

SEQ ID NO: 244 - DNA, 63 bp, Artificial Sequence
  Synthetic
atgcgcggagctgcgctggtgctgctgctgctgctcctgctgctcctggctggcgtgctggcc

SEQ ID NO: 245 - protein, 21 aa, Artificial Sequence
  Synthetic
MRGAALVLLLLLLLLLSPALA

SEQ ID NO: 246 - protein, 4 aa, Artificial Sequence
  Synthetic
KDEL

(19) United States
(12) Patent Application Publication
(10) Pub. No.: US 2010/0305002 A1
(43) Pub. Date: Dec. 2, 2010

(54) Reagents and Methods for Producing Bioactive Secreted Peptides

(75) Inventors: Alex Chenchik, Redwood City, CA (US); Andrei Gudkov, East Aurora, NY (US); Andrei Komarov, San Mateo, CA (US); Venkatesh Natarajan, Cheektowaga, NY (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ROSWELL PARK CANCER INSTITUTE, Buffalo, NY (US)

(21) Appl. No.: 12/768,721

(22) Filed: Apr. 27, 2010

Related U.S. Application Data
(60) Provisional application No. 61/173,122, filed on Apr. 27, 2009.

(51) Int. Cl. C40B 30/06; C12N 15/85; C40B 50/14

(52) U.S. Cl. 506/10; 435/320.1; 506/14

(57) ABSTRACT

This invention discloses reagents and methods for identifying peptides that modulate biological activities in cells, tissues, organs and organisms.

### CROSS-REFERENCES TO RELATED APPLICATIONS

This application claims the benefit of priority from U.S. Provisional Application No. 61/173,122, filed on Apr. 27, 2009, which is explicitly incorporated herein by reference in its entirety for all purposes.

### STATEMENT REGARDING FEDERALLY-SPONSORED RESEARCH OR DEVELOPMENT

This invention was supported in part by grant No. CA60730 from the National Institutes of Health, National Cancer Institute, and grant No. RR02432 from the National Center for Research Resources. The government may have certain rights in this invention.

### BACKGROUND OF THE INVENTION

1. Field of the Invention

This invention relates to reagents and methods for identifying bioactive secreted peptides (BASPs) in animals, particularly humans. Generally, the invention relates to reagents and methods for identifying such BASPs derived from the entire natural proteome or all known bioactive peptides expressed and secreted to the outside of the cell, which act at or upon the cellular membrane. Specifically, the invention provides a plurality of recombinant expression constructs encoding peptide fragments of proteins comprising the natural proteome and known peptides with biological activities and methods for using said constructs to identify specific peptide species having a biological effect when expressed in recipient cells. Also provided by the invention are said peptides useful for the treatment of cancer, neuronal and muscle degeneration, and metabolic, immunological, and infectious diseases.

2. Summary of the Related Art

All aspects of cellular function, including localization, metabolism, proliferation, differentiation, and cell death, among others, involve regulatory proteins that interact and activate specific cellular sensor protein molecules (receptors). The vast majority of cellular control mechanisms regulating these and other aspects of cellular physiology are regulated by mechanisms involving signal transduction through plasma membrane receptors. Thus, developing pharmacological agents that activate or inhibit such regulatory mechanisms could provide an effective approach for treating diseases, disorders, and other pathological disruptions of cellular functions.

The molecules involved in regulating cellular function in nature are predominantly proteins, specifically regulatory molecules interacting with receptors that are also predominantly proteins. There are a number of protein-based drugs, including predominantly antibodies and growth factors, known in the art and approved by government regulators. In all of these cases, however, it has been full-length proteins that have been used as drugs, and these molecules have intrinsic limitations and drawbacks. For example, due to their length and complexity, full-length proteins cannot be chemically synthesized (with the exception of only the simplest of these molecules, such as somatostatin, for example). Accordingly, these proteins must be produced by either mammalian or bacterial cells (i.e., biologics), which have the disadvantages associated with pharmaceutical agents that have been produced from such sources.

An attractive alternative would be to make drugs from peptides, i.e., short amino acid polymers of less than about 100 amino acids, which can be chemically synthesized. Peptides offer unique advantages over small molecule drugs in terms of increased specificity and affinity to targets as a result of their apparent ability to recognize active or biologically relevant sites within a protein target. While the need for peptide drugs was recognized long ago, peptide drugs, particularly peptide drugs derived from the proteome, have been very difficult to identify and develop in the past. This is due to a number of technical problems, including: low chemical stability, low specific activity of peptides compared to proteins, and a lack of efficient methods for screening bioactive peptides with desirable activity to be suitable as pharmacological agents from extremely high complexity peptide libraries. In addition, to be effective as drugs, peptide drug screening should identify molecules that act at the cell surface. Currently available technologies only allow for the functional identification of intracellular peptides, which are not viable drug candidates because they require, inter alia, methods for effectively delivering them inside target cells.

Historically, the first peptide libraries were developed by combinatorial chemical synthesis methods. Concurrent advances in molecular biological methods have facilitated the development of biological peptide libraries. Among them, phage display technology has emerged as a powerful tool for isolating peptide ligands for numerous antibodies, receptors, enzymes, carbohydrates, affinity chromatography, for targeting tumor vasculature, tumor cell types, and more recently, for cancer biomarker discovery and in vivo imaging. While phage display libraries are powerful tools to identify peptides based on in vitro binding to purified target proteins (Livnah et al., 1996, Science 273: 464-71), they are not suitable for isolating peptide modulators of cellular functions in cell based assays due to several of the technical limitations discussed herein.

Since peptides are genetically encoded molecules, peptide-encoding libraries prepared using recombinant genetic methods have been used for screening (Xu et al., 2001, Nature Genet. 27: 23-29; de Chassey et al., 2007, Mol. Cell Proteomics 6: 451-59; Tolstrup et al., 2001, Gene 263: 77-84). However, this technology has been applied for isolating intracellular peptides and has not resulted in peptidic drugs due to difficulties in delivery as discussed herein. Another genetic technology for screening bioactive peptides—genetic suppressor element (GSE) methodology—takes advantage of libraries expressing randomly fragmented pieces of cDNAs (see, e.g., U.S. Pat. Nos. 5,217,889; 5,665,550; 5,753,432; 5,811,234; 5,942,389; 6,060,244; 6,083,745; 6,083,746; 6,197,521; 6,268,134; 6,281,011; 6,326,134; 6,376,241; 6,541,603; and 6,982,313). While GSE libraries carry natural sequences and are therefore enriched for bioactive clones, they are not adapted to be efficiently or effectively screened for secreted peptides. Moreover, not a single excreted peptide has been reported to have been isolated using this technology.

A previously published report on screening secreted molecules was limited to bioactive full-length proteins and did not allow for high-throughput capabilities (Lin et al., 2008, Science 320: 807-11).

Alternative approaches for identifying bioactive molecules have been developed. Over the last decade, the high-throughput (HT) screening approach has gained widespread popularity in drug discovery research. With the advent of automated technologies and development of a wide range of cell-based assays, functional screening of complex small molecule libraries has become routine in the search for pharmacological agents. For example, RNAi screening strategies demonstrate great promise in the identification of therapeutic targets. However, RNAi molecules result in complete or partial loss of all protein functions, whereas peptides, due to their apparent ability to recognize active or biologically relevant sites within a protein target, are likely to interfere with only one of several functions of a target protein, much like a drug. Moreover, recent innovations in peptide design, delivery, and improvement in protease resistance have increased drug development efforts with peptides. Despite these advances and the attractive therapeutic potential of peptides as drugs, progress in developing functional high-throughput screening platforms for peptide drug discovery is lagging.

Thus, there exists a need in the art for developing robust methods for producing libraries of peptide molecules derived from entire proteome of all kingdoms (i.e., eukaryotic, prokaryotic, or viral origin), preferably from known proteins and peptides with known biological activities for producing peptide-derived drugs. There exists a related need to produce such drugs, particularly peptides that bind to, interact with, or otherwise cause phenotypic effects on mammalian, preferably human, cells by interaction with cellular plasma membranes and the receptors and other molecules comprising said cellular membranes.

### SUMMARY OF THE INVENTION

This invention provides reagents and methods for producing libraries of peptide molecules derived from a mammalian, preferably human, proteome for producing peptide-derived drugs, and the peptides produced therefrom. The reagents and methods disclosed herein enable biologically-active secreted peptides (BASPs) to be isolated from proteins comprising the entire natural proteome or known bioactive peptides for any biological activity that can be selected for or against or can be observed as a phenotypic change, either of a biological activity encoded endogenously in a cellular genome or introduced, for example, as a detectable reporter gene (or its expressed encoded protein). Examples of said biological activities include, but are not limited to, cell survival (including selection for and against senescence, apoptosis, and cytotoxicity), metabolism, differentiation, and immune responses. Specific signal transduction pathways assayed using the reagents and methods of the invention include p53, NF-κB, HIF 1 alpha, HSF-1, AP1, differentiation markers, and peptide hormones.

The invention provides reagents for producing libraries of peptide molecules derived from an extracellular mammalian proteome or all known bioactive peptides for producing peptide-derived drugs, and the peptides produced therefrom. As set forth in greater detail herein, the reagents of the invention comprise recombinant expression constructs capable of expressing peptides derived from the extracellular proteome in a eukaryotic cell. Said recombinant expression constructs comprise vector sequences, preferably virus-derived vector sequences, that can be replicated in cells, particularly eukaryotic cells and specifically mammalian cells, and that can comprise a nucleic acid encoding said peptide molecules derived from a mammalian, preferably human, extracellular proteome. In particular embodiments, the vectors are viral vectors, specifically adenovirus, adeno-associated virus, and retrovirus particularly lentivirus. In certain embodiments, plasmid sequences comprise the vector or provide functions (such as an origin of replication and selectable marker sequences) for producing the recombinant expression construct in bacteria or other prokaryotes.

The recombinant expression constructs of the invention further comprise a promoter functional in a eukaryotic, particularly a mammalian and specifically a human cell, preferably positioned 5′ to a site containing at least one and preferably a plurality of restriction enzyme recognition sequences (otherwise known as a multicloning site) into which nucleic acids encoding peptide molecules derived from natural proteins or bioactive peptides can be introduced. In certain embodiments, said promoter is a viral promoter, for example a cytomegalovirus promoter. In other embodiments, the promoter is an inducible promoter that naturally, or as the result of genetic engineering, can be regulated by contacting a cell comprising the recombinant expression vector with an inducing molecule. Inducible promoters are known in the art and include promoters induced by tetracycline or doxicycline or promoters derived from bacterial beta-galactosidase that are induced with X-gal and similar reagents.

The recombinant expression constructs of the invention further comprise nucleic acid encoding a secretion signal positioned 3′ to the promoter and 5′ to the cloning site sequences, wherein the nucleic acids encoding peptide molecules from a mammalian, preferably human, extracellular proteome are introduced to produce a transcript wherein the secretion signal is in-frame with the peptide-encoding sequences. In certain embodiments, the secretion signal is the secreted alkaline phosphatase signal sequence, naturally-occurring or genetically-enhanced interleukin-1 signal sequence, or a hematopoietic cell surface marker signal sequence (e.g., CD14).

The recombinant expression constructs of the invention may further comprise a nucleic acid encoding an oligomerization sequence, particularly a sequence encoding a leucine zipper peptide, which are positioned in the construct either between the secretory protein sequence and the nucleic acids encoding peptide molecules derived from a mammalian, preferably human, extracellular proteome, or positioned 3′ to the nucleic acids encoding peptide molecules derived from a mammalian, preferably human, extracellular proteome, in either case arranged so that the leucine zipper-encoding nucleic acid is introduced into the construct at the proper position and in-frame with the reading frame of the secretory protein sequence and the peptide-encoding nucleic acids.

The recombinant expression constructs of the invention further comprise a nucleic acid encoding a peptide molecule derived from a mammalian, preferably human, extracellular proteome. As provided herein, said nucleic acid encodes a peptide comprising 4 to 100 amino acids, more specifically peptides comprising from 20 to 50 amino acids, and even more specifically from 5 to 20 amino acids. In certain embodiments, said nucleic acids are produced in vitro using computer-assisted solid substrate synthetic methods, wherein a plurality (up to about 10⁶) nucleic acids each having a unique sequence can be prepared. The peptides preferably comprise an overlapping set of peptides from each member of the natural proteins or bioactive peptides and selected to comprise the portion of the proteome represented in the plurality of nucleic acids. In certain embodiments, the plurality of encoded peptide sequences comprise one or more structural or sequence motifs or protein domains or subdomains. Preferably, each such single-stranded nucleic acid is detachably affixed to the solid substrate, and comprises sequences at each of the 5′ and 3′ ends that are complementary to oligonucleotide primers that are used for in vitro amplification. Upon being liberated by chemical treatment from the solid substrate, the plurality of such nucleic acids encoding peptide molecules derived from a mammalian, preferably human, extracellular proteome are amplified and introduced using recombinant genetic methods into the construct at a site ′5 to the promoter and secretory protein portions of the construct. As set forth in more detail below, the primer and vector sequences are arranged so that each of the peptide-encoding nucleic acids is introduced into the construct at the proper position and in-frame with the reading frame of the secretory protein sequence.

In certain embodiments, the recombinant expression constructs comprise additional sequences. In certain of these embodiments, a nucleic acid encoding a peptide sequence that mediates cyclization of the encoded peptide is introduced flanking the nucleic acids encoding peptide molecules derived from a mammalian, preferably human, extracellular proteome, i.e., one such sequence positioned in the construct 5′ and another such sequence positioned in the construct 3′ to the nucleic acids encoding peptide molecules derived from a mammalian, preferably human, extracellular proteome. These sequences are introduced into the construct so that each of the cyclization peptide-encoding nucleic acids is introduced into the construct at the proper position and in-frame with the reading frame of the secretory protein sequence and the peptide-encoding nucleic acids. In certain embodiments, a nucleic acid encoding a transmembrane-localization peptide or protein is positioned in the construct 3′ to the nucleic acids encoding peptide molecules or fusion sequences between peptide sequence and sequence of multimerization domain, and is so that the transmembrane-localizing nucleic acid is introduced into the construct at the proper position and in-frame with the reading frame of the secretory protein sequence and the peptide-encoding nucleic acids. In certain of these embodiments, the transmembrane localization peptide or protein is a transmembrane domain-comprising portion of human PDGF receptor.

The recombinant expression construct of the invention advantageously further comprises a reading-frame selection marker for selecting cells comprising the components of the construct as set forth herein in proper reading frame. In certain embodiments, such markers comprise a selectable marker protein, such as genes encoding drug resistance (e.g., puromycin) that can be used to select for cells comprising constructs wherein the components set forth herein are properly positioned to produce transcripts having the peptide-encoding components in-frame with one another (i.e., without a frameshift mutation).

The skilled worker will also recognize that it is advantageous for the recombinant expression vector of the invention to comprise sequences complementary to oligonucleotide primers useful for in vitro amplification, nucleotide sequencing, or combinations thereof, wherein said primer binding sites do not otherwise interfere with the other functions of the recombinant expression construct. The recombinant expression constructs of the invention can also comprise post-transcriptional regulatory elements, generally positioned 3′ to the peptide-encoding nucleic acid components of the construct. A non-limiting example of such a sequence is the woodchuck hepatitis virus post-transcriptional regulatory element.

The invention also provides cell cultures into which a plurality of recombinant expression constructs are introduced, thereby comprising a library of said constructs in cells wherein the phenotype of the peptide encoded by the construct can be assessed. In certain embodiments, the cells of the cell culture further comprise a second recombinant expression construct encoding a detectable marker protein operatively linked to a promoter regulated by interaction of a cell surface protein and a protein from the extracellular proteome. In these embodiments, expression in the cell of a peptide encoded by one of the plurality of first recombinant expression constructs encoding a peptide molecule derived from known proteins or peptides, preferably bioactive protein and peptides, and regulates expression of the detectable marker protein encoded by the second recombinant expression construct. As provided herein, the detectable marker protein (also called a “reporter gene” or “reporter protein” herein) can encode a selectable biological activity, such as drug resistance. In certain embodiments, the detectable marker protein can produce a detectable signal, such as with green fluorescent protein. Cell cultures useful for the practice of the methods of the invention include any eukaryotic cell, and in certain embodiments can be a yeast cell, a mammalian cell, or a human cell. In certain embodiments, the second recombinant expression construct encodes a detectable marker protein that is operatively linked to a promoter responsive to p53, NF-κB, HIF1alpha, HSF-1, Ap1, a differentiation marker, or a peptide hormone. In alternative embodiments, the cells of the cell culture comprising a library of recombinant expression constructs encoding a peptide molecule derived from a mammalian, preferably human, extracellular proteome are useful according to the methods of the invention for identifying peptides associated with senescence, apoptosis, or cell death, by identifying the members of the plurality of peptides that do not persist in the cells of the library during cell culture (i.e., because cells encoding such peptides do not proliferate).

The invention further provides methods for using cell cultures comprising the libraries of recombinant expression constructs encoding peptide molecules derived from a mammalian, preferably human, extracellular proteome to identify particular peptide-encoding embodiments thereof that produce or mediate a desired cellular phenotype. In certain embodiments, the cell culture is incubated under selective pressure. In alternative embodiments, the cells of the cell culture comprise a second recombinant expression construct encoding a reporter protein that produces a signal, for example, green fluorescent protein, that permits cells comprising reporter-gene activating peptides to be detected and in preferred embodiments, sorted using, for example, fluorescence activated cell sorting (FACS).

The invention also provides bioactive secreted peptides that can be used as drugs, either directly or after modification to improve the stability thereof, for a variety of diseases and disorders. Included among the diseases and disorders for which the methods of the invention provide peptide-based drugs are, without limitation, cancer, immunological diseases (such as, but not limited to, inflammations, allergies, and transplant rejection), cardiovascular diseases, neuronal and muscle degeneration, infection diseases, and metabolic diseases.

The reagents and methods of the invention have several advantages over what was known in the prior art. Natural peptides are expected to be particularly effective in drug discovery inter alia because of their apparent ability to recognize active or biologically relevant sites of protein targets. There are several reasons that can account for the apparent specificity of peptides for active sites. First, most proteins interact with other proteins through several small epitopes, which very often work cooperatively with each other. Cooperative interaction of critical residues in the active center of peptides (usually comprising from between three and ten amino acid residues) leads to a more specific protein-protein interaction than is observed for small molecules (see, e.g., Kay et al., 1998, Drug Discov. Today 8: 370-78). Second, peptide (or protein-protein) binding involves recesses or cavities present in the active or binding sites of the receptor, wherein binding is driven by displacement of water molecules from recesses or cavities in the target molecule (Ringe, 1995, Curr. Opin. Struct. Biol. 5: 825-29). In addition, peptides are unique, highly complex structures comprising a combinatorial set of hydrophobic, basic, acidic, aromatic, amide, and nucleophilic groups that differ from the “chemical space” available in small molecule libraries. Third, because the peptides encoded by the recombinant expression constructs of the invention comprise 4 to 100 amino acids, and more particularly 20 to 50 amino acids, and even more specifically from 5 to 20 amino acids, their interactions with cellular protein targets can be highly specific due to the extended contact surface area. For example, in contrast with G-protein-coupled receptors, small-molecule agonists of the cytokine and growth factor receptor families are difficult to identify because receptor ligand binding sites are found over large areas without significant invaginations (Deshayes, 2005, “Exploring protein-protein interactions using peptide libraries displayed on phage,” in PHAGE DISPLAY IN BIOTECHNOLOGY AND DRUG DISCOVER, pp. 255-82, Sidhu, ed.). It also appears that many cytokine receptors preferentially bind sets of epitopes that resemble “miniproteins” (id.). Certain monoclonal antibody-based drugs, for example, infliximab (Remicade) block the interaction of TNFα with its cognate receptor on B cells and can target these types of “extended” protein interactions very effectively due to their large surface area and structural complexity. It is possible, however, that subdomain-like peptides (comprising about 30 to 50 amino acids) could be as effective as monoclonal antibodies at modulating receptor-ligand interactions, and possess the most suitable characteristics for synthesis and delivery.

Although in nature two interacting proteins can be rather large, protein-protein interaction sites are often present in a single modular domain. It is now well understood that, in most cases, proteins were evolutionarily created by the combinatorial exchange of multiple domains with different specific functions, all acting in concert to contribute to total protein function. Moreover, long peptides (comprising from about 30 to about 50 amino acids) can often effectively mimic the functions of individual domains, and thus supply independent therapeutic functions distinct from those of the holoprotein (Lorens et al., 2000, Mol. Therapy 1: 438-47; Watt, 2006, Nat. Biotechnol. 24: 177-83; Santonico et al., 2005, Drug Discov. Today 10: 1111-17). For example, systematic analyses of ligand-receptor interactions by alanine scanning mutagenesis has revealed that receptor-binding epitopes, even in comparatively small molecules such as cytokines, are organized into exchangeable modules (domains), and at least two sites (site I and site II) in many cytokines and growth factors lead to dimerization and activation of receptors (Schooltink and Rose-John, 2005, Comb. Chem. High Throughput Screen. 8: 173-79).

Peptide ligands, as modulators of cellular functions, can also be powerful tools for target validation in the drug discovery process. Identification of therapeutic targets currently relies more on observation than on experimental methods. Human genetics, SNP analysis, mapping of protein-protein interactions, expression profiling, and proteomics, when combined with clinical studies, establish correlations between mutations, protein interactions or expression levels, and disease. A correlation is not a causal link, however, and thus the putative targets identified by these technologies must be subsequently validated. The use of peptides in phenotypic assays has two considerable advantages. First, these reagents might inhibit or activate the function of their cognate target proteins; this advantage enhances opportunities to identify drug targets and reveal new mechanisms of action. Second, target validation can be more quickly achieved with peptides than with gene knockouts, and the use of peptides does not depend on the stability of protein targets, as do siRNAs knockdowns. Moreover, peptides actually offer a better model of drug action; a peptide will probably interfere with only one of several functions of a target protein, much like a drug, whereas genetic knockout or knockdown will result in complete or partial loss of all protein functions (Baines and Colas, 2005, Drug Discov. Today 11: 334-41).

In addition, the methods of the invention are capable of distinguishing between autocrine and paracrine events. All previous attempts to isolate peptide-encoding sequences by functional genetic screening were made with the libraries of intracellular peptides. These approaches did not allow for the identification of pharmacologically feasible peptides expected to act through the cell surface, and not requiring intracellular penetration. The inclusion in the recombinant expression constructs of the invention of a secretory peptide leader sequence at the amino terminus directs the newly-translated peptide product to the endoplasmic reticulum (ER) or Golgi apparatus in the transformed cells. Importantly, this allows the bioactive peptides to cause a biological effect when functional interaction with their cognate targets occurs intracellularly, i.e., between the peptide and a specific receptor already in ER, both of them meeting during processing along protein secretory pathway. This feature results in stronger autocrine biological effects than paracrine effects, making it more likely that peptide-producing cells are identified; this has been verified by detected abrogation of biological activity in constructs lacking the secretory leader peptide-encoding sequences.

The methods of the invention also overcome the problem of excessive complexity encountered using conventional random sequence peptide libraries. The enormous complexity of random peptide libraries results in the problem of practical handling large-scale screenings. Instead of random fragment libraries, the methods of the invention use a rational design-based library, wherein the peptides encoded by the library are derived from peptides, preferably overlapping peptides from proteins comprising the extracellular proteome. These include proteins from blood (hormones, growth factors, cytokines, etc.), cell-cell interactions (integrins, other molecular junctions, receptors of immunocytes, stroma, etc.), extracellular matrix proteins and pathogens/parasites (viruses, bacteria, protozoan parasites, etc.). In common among these sources is that effector molecules are encoded by genomes of existing organisms, suggesting that the extracellular proteome contains the majority of cell surface receptor recognition patterns and therefore provides an ideal source for bioactive secreted peptides of the invention.

The methods of the invention also provide peptides, particularly in embodiments comprising leucine zipper dimers, trimers, or oligomers, for enhancing the biological effects of the peptides encoded in the recombinant expression construct library. Short peptides can have weaker biological effects than full-length proteins due to less rigid tertiary structure resulting in lower affinity to the substrates. Using leucine zipper technology increases the likelihood of identifying peptides in the library from the extracellular proteome that can act as agonists for cell surface receptors. Surprisingly, said peptides can also act as antagonists when expressed in the absence of leucine zipper sequences, presumably due to binding at the same or similar sites and blocking natural aggregation of said receptors that facilitates transmembrane signaling.

The methods of the invention also have the advantage over traditional methods for identifying bioactive peptides that the methods are capable of identifying both positively-selected and negatively-selected phenotypes and peptides. In order to select bioactive secreted peptides that are not associated with growth advantages (e.g., such peptides causing cell differentiation, growth arrest, activation of signaling pathway that is not associated with growth alterations, specifically toxic for the cells of choice), the methods of the invention rely on monitoring relative representation of different library clones in selected cell populations. These embodiments of the claimed methods use high-throughput sequencing of PCR-rescued library inserts or specific sequence tags or barcodes introduced to label each individual clone, wherein appropriate structural elements have been introduced into vectors. Computational analysis of the frequency of specific sequence tags isolated from cell populations before and after growth of cells after introduction of a plurality of BASP-encoding recombinant expression constructs of the invention permits identification of those clones having a representational frequency in the plurality that reliably changes indicative of their specific biological function, including those that cause growth suppression or cell killing.

Specific preferred embodiments of the present invention will become evident from the following more detailed description of certain preferred embodiments and the claims.

### BRIEF DESCRIPTION OF THE DRAWINGS

FIG. 1 is a schematic presentation of the vector map for expression of secreted peptides in free (monomer), dimer (leucine zipper), trimer (leucine zipper), cyclic (EFLIVIKS dimerization domain), and as a fusion product with a transmembrane domain, albumin, or Fc with an upstream secretion signal.

FIG. 2 shows the general design and nucleotide sequence of the pRP-CMV-HTS Peptide (Protein) Expression/Secretion Vector (SEQ ID NO: 1) for cloning linear peptides in BpiI sites. Primers shown in FIG. 2 are: Fwd-CMV12 (SEQ ID NO: 2), Fwd-CMV43 (SEQ ID NO: 3), Gex1 (SEQ ID NO: 4), GexSeq (SEQ ID NO: 5), Gex2 (SEQ ID NO: 6), Rev-WPRE60 (SEQ ID NO: 7), and Rev-WPRE90 (SEQ ID NO: 8). Cloning sites are denoted with nucleotides in lowercase letters.

FIG. 3 shows the nucleotide sequence of the Linear Peptide Cassette (after cloning a 20aa peptide insert into the BpiI sites of the pRP-CMV-HTS vector) (SEQ ID NO: 9), as well as nucleotide sequences of primers Gex1 (SEQ ID NO: 4), GexSeqCC (SEQ ID NO: 10), GexSeqA (SEQ ID NO: 11), and Gex2 (SEQ ID NO: 6). Cloning sites are denoted with nucleotides in lowercase letters.

FIG. 4 shows the nucleotide sequence of the LeuZip Dimer Peptide Cassette (after cloning a 20aa peptide insert into the BpiI sites of the pRP-CMV-LeuZipD-HTS vector) (SEQ ID NO: 12), as well as nucleotide sequences of primers Gex1 (SEQ ID NO: 4), GexSeqCC (SEQ ID NO: 10), GexSeqA (SEQ ID NO: 11), and Gex2 (SEQ ID NO: 6). Cloning sites are denoted with nucleotides in lowercase letters.

FIG. 5 shows the nucleotide sequence of the LeuZip Trimer Peptide Cassette (after cloning a 20aa peptide insert into the BpiI sites of the pRP-CMV-LeuZipT-HTS vector) (SEQ ID NO: 13), as well as nucleotide sequences of primers Gex1 (SEQ ID NO: 4), GexSeqCC (SEQ ID NO: 10), GexSeqA (SEQ ID NO: 11), and Gex2 (SEQ ID NO: 6). Cloning sites are denoted with nucleotides in lowercase letters.

FIG. 6 shows the nucleotide sequence of the Cyclic Peptide Cassette (after cloning a 20aa peptide insert into the BpiI sites of the pRP-CMV-Cyc-HTS vector) (SEQ ID NO: 14), as well as nucleotide sequences of primers Gex1 (SEQ ID NO: 4), GexSeqCY (SEQ ID NO: 15), GexSeqA (SEQ ID NO: 11), and Gex2 (SEQ ID NO: 6). Cloning sites are denoted with nucleotides in lowercase letters.

FIG. 7 shows the nucleotide sequence of the PDGF Transmembrane Domain Fusion Cassette (after cloning a 20aa peptide insert into the BpiI sites of the pRP-CMV-PDGFtm-HTS vector) (SEQ ID NO: 16), as well as nucleotide sequences of primers Gex1 (SEQ ID NO: 4), GexSeqA (SEQ ID NO: 11), and Gex2 (SEQ ID NO: 6). Cloning sites are denoted with nucleotides in lowercase letters.

FIG. 8 shows the nucleotide sequence of Design 1 of the Oligo Pool for peptide library construction (SEQ ID NO: 17), as well as nucleotide sequences for primers FwdPool-PL1 (SEQ ID NO: 18) and RevPool-PL1 (SEQ ID NO: 19). Cloning sites are denoted with nucleotides in lowercase letters.

FIG. 9 is a flowchart of computational tools for the prediction of a comprehensive set of human extracellular proteins and domains.

FIG. 10 is a graphical depiction of autocrine and paracrine activation of reporter gene expression in cells comprising NF-κB-reporter gene constructs.

FIG. 11 is an outline of the screening assay used for NF-κB modulators by transduction of the lentiviral peptide library into reporter cells, selection by FACS of cell fractions displaying modulation of the reporter gene, and identification of all positive peptide hits in the selected cell fractions by HT sequencing (in contrast to the conventional procedure of isolating and analyzing a limited number of single cell clones).

FIG. 12 is a diagrammatic representation of 50K lentiviral ligand peptide library construction. Peptide templates are synthesized on the microarray surface, detached, amplified by PCR, digested, and cloned into the lentiviral vectors with pR-CMV-S3 backbone. The library is packaged into pseudoviral particles in HEK293T cells.

FIG. 13 is a map of the lentiviral secreted vector pR-CMV-S3-TNF. Expression of control TNFα (or peptide) is driven by the CMV promoter. The secreted alkaline phosphatase (SEAP) signal sequence enables secretion of protein/peptides. In the lentiviral peptide cassette, BamHI and EcoRI restriction sites between the SEAP signal sequence and peptide insert allow cloning of leucine zipper dimerization sequence.

FIG. 14 is an outline of the screening assay used for NF-κB modulators by transduction of the lentiviral peptide library into reporter cells, selection by FACS of cell fractions displaying modulation of the reporter gene, and identification of all positive peptide hits in the selected cell fractions by single cell cloning in multiwell plates and conventional sequencing.

FIG. 15 is a photomicrograph of NF-κB-reporter cells secreting TNF and NF-κB-reporter cells without secretion were mixed at 1:10K, and plated with (panels A, B) or without (panels C, D) agar overlay. Autocrine activation of TNF secreting cells induced the reporter cells to become GFP-positive without affecting bystanders.

FIG. 16 shows enrichment of NF-κB agonists only in the GFP+ cell fraction with the test cytokine library. NF-κB-GFP reporter cells were infected with the test 10K cytokine library. After two rounds of FACS sorting, genomic DNA was isolated, and the inserts were rescued by PCR using primers specific to each cytokine Lanes A1, A2, and A3 represent the gene-specific PCR products for each cytokine using genomic DNA from total, GFP-positive (GFP+), and GFP-negative (GFP−) cell fractions.

FIG. 17 is a graphical depiction of high-throughput screening methods of the invention using extracellular proteome-encoding recombinant expression constructs, selection, and lead candidate validation.

FIG. 18 shows the frequency of GFP-positive clones in 293-NFκB-GFP reporter cells transduced with four different 50K secreted 20aa-long (lower panels) and 50aa-long (upper panels) peptide libraries after two rounds of FACS sorting.

FIG. 19 depicts amino acid sequences, structures, and agonist efficacy of peptides furin (26-75) (SEQ ID NO: 20), RTN3 reticulon 3 (2357-2503) (SEQ ID NO: 21), apolipoprotein F (121-170) (SEQ ID NO: 22), apolipoprotein F (121-170, with deletion) (SEQ ID NO: 23), apolipoprotein F (141-190) (SEQ ID NO: 24), cartilage oligomeric matrix protein (429-478) (SEQ ID NO: 25), cartilage oligomeric matrix protein (439-458) (SEQ ID NO: 26), apolipoprotein F (151-180) (SEQ ID NO: 27), and cholecystokinin (95-115) (SEQ ID NO: 28), where were identified in the primary screen of NF-κB effectors in 293-NFκB-GFP reporter cells with a set of 50K secreted peptide libraries. Homology regions between different peptide clones are indicated in bold face or by double-underlining.

FIG. 20 shows the results of 293-NFκB-GFP reporter cells transduced with 50K 20aa (lower panels) or 50aa (upper panels) BASP libraries and sorted by FACS (after two rounds of sorting) for each of the libraries comprising different embodiments of the extracellular proteome-derived peptides.

FIG. 21 shows the results of screening BASP libraries for elements modulating activity of indicated signal transduction pathways. Note that cells with activated p53 have different morphology and do not proliferate.

FIG. 22 is a schematic diagram of an HT viability screen with an updated NCI-60 cancer cell line panel, wherein the screen comprises the steps of constructing a pooled lentiviral BASP library, performing HTS of cytotoxic BASP constructs using a 50K BASP library, rationally designing and constructing primary hits and their mutant 50K BASP sublibraries, confirming and optimizing the viability screen with the 50K BASP hit sublibraries in a pooled format, developing a synthetic BASP hit mimic compound library, performing a secondary round of the validation viability screen in an arrayed format with a BASP compound library, and then data mining and depositing in the DTP NCI-60 database.

FIG. 23 shows the structure of the BASP expression cassette in the pBASP lentiviral vector, along with the mechanism of autocrine activation of death receptors with genetic or synthetic BASP constructs. The pre-pro-BASP design mimics the typical pre-pro-peptide structure of most secreted cytokines and growth factors, which are processed with Sec- and Furin-type proteases and secreted through a conventional ER-Golgi pathway to the extracellular space. In the figure, “Pre” is the consensus secretion signal MRSLSVLALLLLLLLAPASAA (SEQ ID NO: 29), “Pro” is a SUMO or thioredoxin “transport” module, “Peptide” is a 4-20 amino acid rationally designed peptide, “Linker” is the flexible amino acid flexible GGGSGGGSGG (SEQ ID NO: 30), and “LeuZip” is the pLI-GCN4 parallel tetrameric alpha-helical module (Li et al., 2006, J. Mol. Biol. 361: 522-36).

FIGS. 24A and 24B show the general design and nucleotide sequence, respectively, of vector pRPA2-C-SS5-LZ4+8-HTS (SEQ ID NO: 31), a standard vector with not fully characterized secretion properties. Also shown in FIG. 24B are nucleotide sequences for primers Fwd-CMV12 (SEQ ID NO: 2), Fwd-CMV43 (SEQ ID NO: 3), Gex1MS (SEQ ID NO: 32), GexSeqP (SEQ ID NO: 33), and Gex2 (SEQ ID NO: 6), as well as amino acid sequences of the SS5 signal sequence (SEQ ID NO: 34) and the LeuZip tetramerization sequence with flanking 8aa linker and BamHI site (SEQ ID NO: 35). Cloning sites are denoted with nucleotides in lowercase letters.

FIGS. 25A and 25B show the general design and nucleotide sequence, respectively, of vector pRPA2cyto-C-LZ4+8-HTS (SEQ ID NO: 36), a control vector without a secretion signal for transport of tetrameric peptides to the cytoplasm. Also shown in FIG. 25B are nucleotide sequences for primers Fwd-CMV12 (SEQ ID NO: 2), Fwd-CMV43 (SEQ ID NO: 3), Gex1MS (SEQ ID NO: 32), GexSeqP (SEQ ID NO: 33), and Gex2 (SEQ ID NO: 6), as well as the amino acid sequence of the LeuZip tetramerization sequence with flanking Baa linker and BamHI site (SEQ ID NO: 35). Cloning sites are denoted with nucleotides in lowercase letters.

FIGS. 26A and 26B show the general design and nucleotide sequence, respectively, of vector pRPA3-C-SS5-AviTag-Furin-LZ4+8-HTS (SEQ ID NO: 37), a vector with an AviTag pre-pro-peptide to be processed by Furin in the trans-Golgi before secretion. Also shown in FIG. 26B are nucleotide sequences for primers Fwd-CMV12 (SEQ ID NO: 2), Fwd-CMV43 (SEQ ID NO: 3), Gex1MS (SEQ ID NO: 32), GexSeqP (SEQ ID NO: 33), and Gex2 (SEQ ID NO: 6), as well as amino acid sequences of the SS5 signal sequence with AviTag and Furin sequences (SEQ ID NO: 38) and the LeuZip tetramerization sequence with flanking Baa linker and BamHI site (SEQ ID NO: 35). Cloning sites are denoted with nucleotides in lowercase letters.

FIGS. 27A and 27B show the general design and nucleotide sequence, respectively, of vector pRPA4-C-SS5-SUMO-Furin-LZ4+8-HTS (SEQ ID NO: 39), a vector with a SUMO protein carrier to be processed by Furin in the trans-Golgi before secretion. Also shown in FIG. 27B are nucleotide sequences for primers Fwd-CMV12 (SEQ ID NO: 2), Fwd-CMV43 (SEQ ID NO: 3), Gex1MS (SEQ ID NO: 32), GexSeqP (SEQ ID NO: 33), and Gex2 (SEQ ID NO: 6), as well as amino acid sequences of the SS5 signal sequence with SUMO and Furin sequences (SEQ ID NO: 40) and the LeuZip tetramerization sequence with flanking Baa linker and BamHI site (SEQ ID NO: 35). Cloning sites are denoted with nucleotides in lowercase letters.

FIGS. 28A and 28B show the general design and nucleotide sequence, respectively, of vector PRPA5-C-SS5-LZ4+8-HTS-TEV-ENT-PDGFtm (SEQ ID NO: 41), a cell surface display vector for leucine zipper tetrameric peptides. Also shown in FIG. 28B are nucleotide sequences for primers Fwd-CMV12 (SEQ ID NO: 2), Fwd-CMV43 (SEQ ID NO: 3), Gex1MS (SEQ ID NO: 32), GexSeqP (SEQ ID NO: 33), and Gex2 (SEQ ID NO: 6), as well as amino acid sequences of the SS5 signal sequence (SEQ ID NO: 34) and the LeuZip tetramerization sequence with flanking Baa linker, TEV, ENT, PDGFtm, and BamHI site sequences (SEQ ID NO: 42). Cloning sites are denoted with nucleotides in lowercase letters.

FIGS. 29A and 29B show the general design and nucleotide sequence, respectively, of vector PRPA6-C-SS5-Fc+8-HTS-TEV-ENT-PDGFtm (SEQ ID NO: 43), a cell surface display vector for Fc dimeric peptides. Also shown in FIG. 29B are nucleotide sequences for primers Fwd-CMV12 (SEQ ID NO: 2), Fwd-CMV43 (SEQ ID NO: 3), Gex1MS (SEQ ID NO: 32), GexSeqP (SEQ ID NO: 33), and Gex2 (SEQ ID NO: 6), as well as amino acid sequences of the SS5 signal sequence (SEQ ID NO: 34) and the Fc sequence with flanking Baa linker, TEV, ENT, PDGFtm, and BamHI site sequences (SEQ ID NO: 44). Cloning sites are denoted with nucleotides in lowercase letters.

### DETAILED DESCRIPTION OF THE INVENTION

The reagents and methods provided by this invention address and overcome limitations in the prior art that have hindered or prevented peptide-based drug development. Historically, combinatorial chemical synthesis methods have enabled the development of the first peptide libraries synthesized in different formats (soluble or attached to beads, resins, or other solid supports). Concurrent advances in molecular biological methods have facilitated the development of biological peptide libraries (Mersich and Jungbauer, 2008, J. Chromatography 861: 160-70). Traditionally, expression libraries of full-length proteins, domains, or small peptide fragments have been used to discover modulators of cellular functions. Functional screening with plasmid or viral cDNA libraries has become routinely used over the last two decades in the discovery of novel oncogenes, receptor ligands, and cell signaling modulators, in the study of protein-protein interactions (two hybrid system), and in the isolation of beneficial protein mutants by combinatorial or site-directed mutagenesis (see, e.g., Michiels et al., 2002, Nat. Biotechnol. 20: 1154-57; Chanda and Caldwell, 2003, Drug Discov. Today 8: 168-74; Ying, 2004, Mol. Biotechnol. 27: 245-52; Yashiroda et al., 2008, Curr. Opin. Chem. Biol. 12: 55-59). cDNA libraries of secreted cytokines and extracellular proteins have been successfully used for the discovery of novel receptor modulators (Lin et al., 2008). Random fragment library screening using genetic suppressor elements have been used to identify both intracellular truncated proteins and antisense RNAs that act as dominant effectors or inhibitory molecules modulating cell signaling pathways (Roninson et al., 1995, Cancer Res. 55: 4023-25; Delaporte et al., 1999, Ann. N.Y. Acad. Sci. 886: 187-90).

Also known in the prior art are retroviral expression peptide libraries containing random sequences (Lorens et al., 2000; Xu et al., 2001; Tolstrup et al., 2001). Retroviral libraries expressing cyclic peptides flanked with EFLIVKS (SEQ ID NO: 45) dimerization sequences have been successfully used in functional screens of cell cycle inhibitors (Xu et al., 2001). In spite of the high potential for the discovery of novel drug targets and the development of novel peptide drugs, GSE and random peptide intracellular expression libraries have not had broad application, mainly due to difficulties in construction, low efficacy, and complicated HT functional screening methodology.

Among peptide libraries, phage display technology has been most widely employed, both in biotechnology industries and academic laboratories (Kay et al., 1998; PHAGE DISPLAY: A PRACTICAL APPROACH, 2003, Clackson and Lowman, eds.; PHAGE DISPLAY IN BIOTECHNOLOGY AND DRUG DISCOVERY, 2005, Sidhu, ed.; Dennis, 2005, “Selection and screening strategies,” in PHAGE DISPLAY IN BIOTECHNOLOGY AND DRUG DISCOVERY, pp. 143-64, Sidhu, ed.). This technology is based on peptides or proteins being capable of being fused to phage coat proteins without loss in the phage's infectivity; these proteins are also accessible for molecular interactions. In contrast to synthetic peptide libraries, biological libraries are inexpensive to construct, being readily amplifiable in bacteria. Phage libraries displaying of 10⁸-10¹⁰ different peptides (a complexity far surpassing combinatorial synthetic peptide libraries) can be readily constructed from degenerate oligonucleotides (PHAGE DISPLAY: A PRACTICAL APPROACH, 2003; PHAGE DISPLAY IN BIOTECHNOLOGY AND DRUG DISCOVERY, 2005). Phage display technology has been used for isolating several peptide antagonists and agonists for different classes of cell surface receptors (Miller, 2000, Drug Discov. Today 5: S77-83; Schooltink and Rose-John, 2005; Kallen et al., 2000, Trends Biotechnol. 18: 455-61; Deshayes, 2005). One class of successful targets identified using phage display technology is the integrins, a family of heterodimeric proteins involved in binding various extracellular matrix proteins (e.g., fibronectin, laminin) Biologically-active peptides that bind to the platelet integrin gpIIb/IIIa and inhibit platelet aggregation have been isolated from a library of cyclized peptides possessing the CXXRGDC (SEQ ID NO: 46) motif (O'Neil et al., 1992, Proteins 14: 509-15). Another example of peptides isolated using phage display technology are peptides that bind to the thrombin receptor of whole platelets; such platelets have been shown to inhibit platelet aggregation at a ten-fold lower concentration than previously reported antagonists of the thrombin receptor (Doorbar and Winter, 1994, J. Mol. Biol. 244: 361-69). Another example of peptides isolated using phage display technology are selectins, a class of molecules that bind carbohydrates and glycoproteins on cell surfaces. E-selectin was used to screen a phage library, leading to isolation of peptides with nanomolar dissociation constants that inhibit neutrophil cell adhesion in vitro and neutrophil cell migration to sites of inflammation in vivo (Martens et al., 1995, J. Biol. Chem. 270: 21129-36). Peptide ligands for the erythropoietin (EPO) receptor were discovered in a library of cyclized combinatorial peptides (Wrighton et al., 1996, Science 273: 458-64). One particular 14-mer peptide, while lacking any obvious primary structural similarity to EPO, bound as a dimer within the receptor binding pocket (Livnah et al., 1996), was a potent agonist in cell assays and in mice, and could compete with EPO binding to its receptor with an IC₅₀ of 2 nM (Wrighton et al., 1996, Nat. Biotechnol. 15: 1262-65). Peptides (14-mers) that bind to the thrombopoietin (TPO) receptor as a dimer with a 2 nM dissociation constant and are potent agonists of the TPO molecule itself have also been recently described (Cwirla et al., 1997, Science 276: 1696-99)

Most protein therapeutics currently on the market are agonists, and thus are needed only in small quantities in order to activate their targeted receptor. In addressing cancer and inflammation, however, antagonists are most commonly sought in order to prevent the activation of receptors involved in disease progression (Ladner et al., 2004, Drug Discov. Today 9: 525-29). Many such receptors (e.g., the interleukin-1 receptor, IL-1R) are activated by binding to protein or peptide ligands. Phage-derived peptide antagonists have been developed that bind to the IL-1R and that have both antagonist activity (IC₅₀=2 nM) in vitro and the ability to block IL-1-driven responses in human cells (Yanofsky et al., 1996, Proc. Natl. Acad. Sci. U.S.A. 93:

7381-86; Deschyes et al., 2002, Chem. Biol. 9: 495-505). Hetian et al., 2002 (J. Biol. Chem. 277: 43137-42) used the display of multiple gIIIp peptides on M13 phages to identify the HTMYYHHYQHHL peptide (SEQ ID NO: 47), which binds to the vascular endothelial growth factor (VEGF) receptor domain-containing receptor kinase. This peptide slows the growth of breast carcinoma tumors in mice (Hetian et al., 2002; Pan et al., 2002, J. Mol. Biol. 316: 769-87). Karasseva et al. (2002, J. Prot. Chem. 21: 287-96) identified a peptide that binds to recombinant human ErbB-2 tyrosine kinase receptor, which is implicated in many human malignancies. Although phage display technology has successfully been used to discover specific, high-affinity peptide ligands for a wide range of different receptors, the probability of identifying peptide ligands with agonist or antagonist activity through random screening appears to be much lower than for binding peptides (Mersich and Jungbauer, 2008; Watt, 2006; Santonico et al., 2005).

Despite these impressive achievements, phage display libraries are not currently considered as a promising approach for functional screening in cell-based assays (PHAGE DISPLAY: A PRACTICAL APPROACH, 2003; PHAGE DISPLAY IN BIOTECHNOLOGY AND DRUG DISCOVERY, 2005) due to the low biological activity of the displayed peptides at the phage concentration used in the screen and the high level of non-specific binding to the cell surface. In addition, random peptide phage display libraries possess a complexity that is too high, even for short peptides (for example, peptides comprising six amino acids require 20⁶ peptides (6.4×10⁶), while 10-mers require 20¹⁰ or 1.02×10¹³ peptides), and as a result they cannot be effectively used in cell-based assays, which are limited in terms of the cell numbers used in the screen (less than 1×10⁸ cells).

Compared with random peptide libraries, protein domains (ranging from 30 amino acids to 300 amino acids in length) and subdomains (being from 20 amino acids to 70 amino acids in length) of natural proteins have been optimized by evolution for stable folding. In addition, the bioactive peptide folds have undergone natural selection for high potency (key contact residues to impart function), in vivo stability (against proteases), and low immunogenicity (Li et al., 2006; Lader and Ley, 2001, Curr. Opin. Biotechnol. 12: 406-10). Since these evolutionarily conserved domains are modular, they often comprise independent functional motifs with distinct binding, activation, repression, or catalytic activities. These units are combined in a modular fashion to fine-tune the function of the full protein. Based on several distinct modeling approaches, all proteins from natural species may be derived from a combinatorial assembly of only about 12,000 domain models (families) curated in NCBI's Conserved Domain Database (CDD) (Marchler-Bauer et al., 2009, Nucl. Acids Res. 37: D205-10). Based on the 12,000 domains described to date, only a limited set of highly structured domains with stable folds has been significantly evolved in about 2,500 superfamily clusters. It is interesting to note that the distribution of amino acids in different stable folds (domain superfamilies) is not random when amino acids are considered within their chemical groups (Baud and Karlin, 1999, Proc. Natl. Acad. Sci. U.S.A. 96: 12494-99).

Moreover, similar fold structures can be encoded by highly divergent sequences because biological molecules often recognize shape and charge rather than merely the primary sequence (Watt, 2006; Yang and Honig, 2000, J. Mol. Biol. 301: 691-711). A good example of structural domain homology can be found in the nuclear hormone receptor superfamily. These proteins possess a structurally conserved ligand-binding domain that binds rather specifically to a wide range of hydrophobic molecules as diverse as steroid and thyroid hormones, retinoids, fatty acids, prostaglandins, leukotrienes, bile acids, and xenobiotics (Koch and Waldmann, 2005, Drug. Discov. Today 10: 471-83). Furthermore, as demonstrated by Anantharaman et al. (2003, Curr. Opin. Chem. Biol. 7: 12-20), the same domain folds can have differing functional roles in a number of higher organisms. Considering that most peptide drugs developed thus far are of human origin, only a small fraction of the true diversity of naturally occurring bioactive peptides has been sampled in the search for new drug candidates. To fully exploit the rich diversity of peptides encoding domain/subdomain structures, it is possible to create comprehensive peptide libraries that comprise all sequence motifs found in the natural kingdom. Because there are a limited number of extracellular protein subdomain structures in nature, diverse libraries containing several hundred thousand different subdomains constitute virtually all of the available classes of protein fold structures and will provide a rich source of peptides that could modulate receptor-mediated cell signaling.

The invention provides recombinant expression constructs comprising vector sequences, a promoter functional in eukaryotic, particularly mammalian and specifically human cells, a protein secretory “signal” sequence, a plurality of nucleic acid sequences encoding peptides from 4 to 100 amino acids in length, more particularly 20 to 50 amino acids in length, and even more specifically from 5 to 20 amino acids, and positioned in-frame with the signal sequence, and optionally in alternative embodiments one, two, or three copies of a sequence such as a leucine zipper sequence that produces monomer, dimmer, or trimer embodiments of the encoded peptide sequence, or a cyclization sequence, or a transmembrane domain sequence. Non-limiting examples of constructions of the invention are arranged as set herein.

Certain embodiments of the invention provide lentiviral vectors that secrete peptides into the extracellular space, wherein the vector comprises a protein secretory sequence, or “signal” sequence, which in particular embodiments is the signal sequence of alkaline phosphatase (SEAP), which was found to consistently mediate secretion of all positive control proteins (TNFα, IL-1β, and flagellin). Several approaches exist for the design of BASP libraries to provide effective secretion of bioactive secreted peptides into the extracellular space. For example, BASP libraries can be designed to yield pro-peptides, which can be processed by convertases (e.g., furin, PC1, PC2, PC4, PC5, PACE4, and PC7). Alternatively, a protease cleavage site for a site-specific protease (e.g., Factor IX or Enterokinase) can be included between the pro sequence and the bioactive secreted peptide sequence, and the pro-peptide can be activated by the treatment of cells with the site-specific protease.

In another embodiment, effective secretion may be provided by using membrane anchoring. Receptor ligands, such as TNFα, are attached to the membrane through a transmembrane domain and such ligands activate their corresponding receptor through cell-cell interactions or after shedding by proteases (like metalloprotease) or other stimuli. This approach has been used for the cell surface display of antibodies and peptides.

In another embodiment, effective secretion may be provided by removal of carbohydrate groups from the peptides. At least 50% of secreted peptides and proteins are glycosylated. While glycosylation of proteins is important for correct folding and possibly secretion, carbohydrate groups are large and rigid, and may block the activity of peptides. Thus, the carbohydrate group could be removed by processing by adding N-glycanase to culture media.

The recombinant expression constructs of the invention can be used in high-throughput screening (HTS) assays using lentiviral peptide libraries in a pooled format. In certain embodiments, these assays exploit the advantages of high-throughput (HT) sequencing platforms to rapidly identify enriched peptide inserts, inter alia, in FACS-selected cell fractions wherein particular members of the library are identified by activation of a detectable reporter gene. The identities of the peptides in the sorted population are then ascertained by rescue of the peptide inserts from the vectors integrated into the cellular genomes by, inter alia, polymerase chain reaction (PCR) amplification and cloning thereof. To this end, as illustrated above, the constructs of the invention comprise primer binding sites (designated Gex1, Gex2, and GexSeq primer-binding sites herein) (or alternatively comprise a unique restriction site for ligation of the adaptor to the Gex binding sequence) flanking the peptide expression cassette. This vector design permits amplification and HT sequencing. As set forth herein, in certain embodiments of the invention, the construct also comprises a unique restriction site internally (BbsI) to clone the peptide inserts directly or to introduce additional cassettes for expression of constrained peptides or peptides in the scaffold of other proteins.

In certain embodiments of the invention, the promoter functional in eukaryotic, particularly mammalian and specifically human cells, is a cytomegalovirus promoter. In specific embodiments, this promoter is altered as set forth herein to provide tetracycline (tet)-dependent regulation of secreted peptide expression, using a well-characterized CMV-TetO7 promoter (Clonetech, Mountain View, Calif.). Tet-regulated expression is particularly useful for HTS of toxic or growth arrest-inducing peptides and receptor agonists with feed-back regulation of induced cell signaling.

Most cytokine mimetics identified by phage display approaches bind to the receptor as dimers or trimers; for example, the TRAIL ligand (Li et al., 2006) is trimeric. In certain embodiments of the invention, recombinant expression constructs comprise in the alternative free linear peptides and “constrained” peptides comprising sequences that form dimers or trimers of each of the peptides encoded in the library. These embodiments seek to interrogate the complexity and diversity of ligand-receptor interactions, by comparing the functional activity of free linear peptides and constrained peptides exposed in different protein scaffolds. In these embodiments, nucleotide sequences encoding leucine zipper dimerization and trimerization domains were introduced into the recombinant expression constructs of the invention downstream of the signal sequence (into the BbsI site, for example, as shown herein). Leucine zipper cassettes are designed with an internal Bbs I site to allow for in-frame cloning of peptide libraries downstream of the leucine zipper sequences.

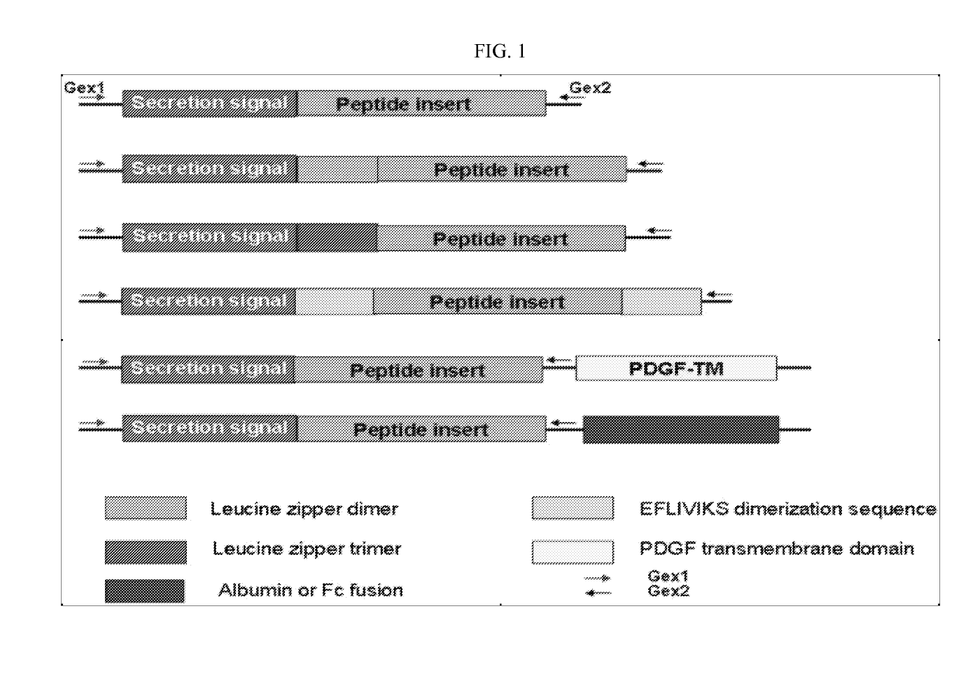

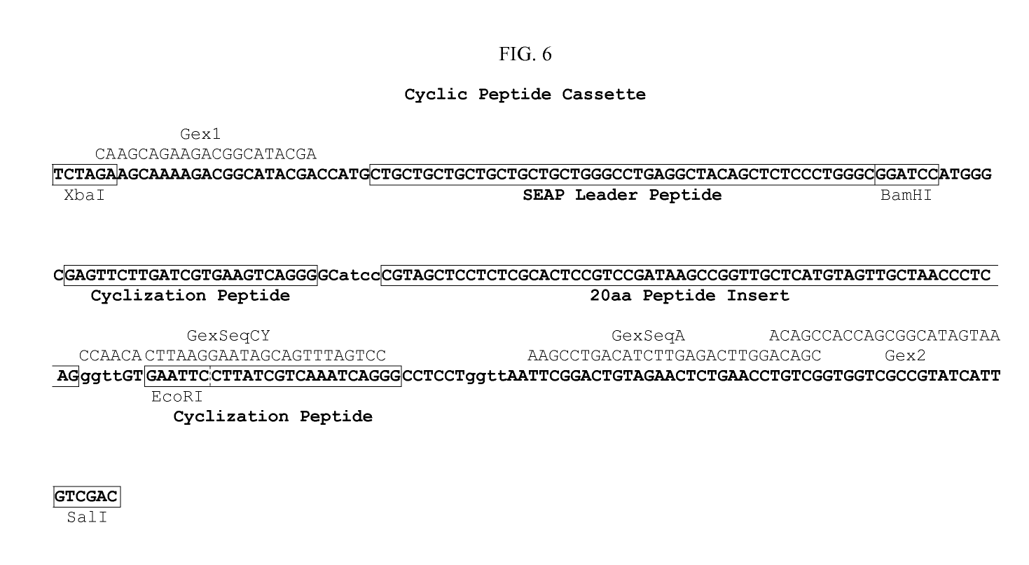

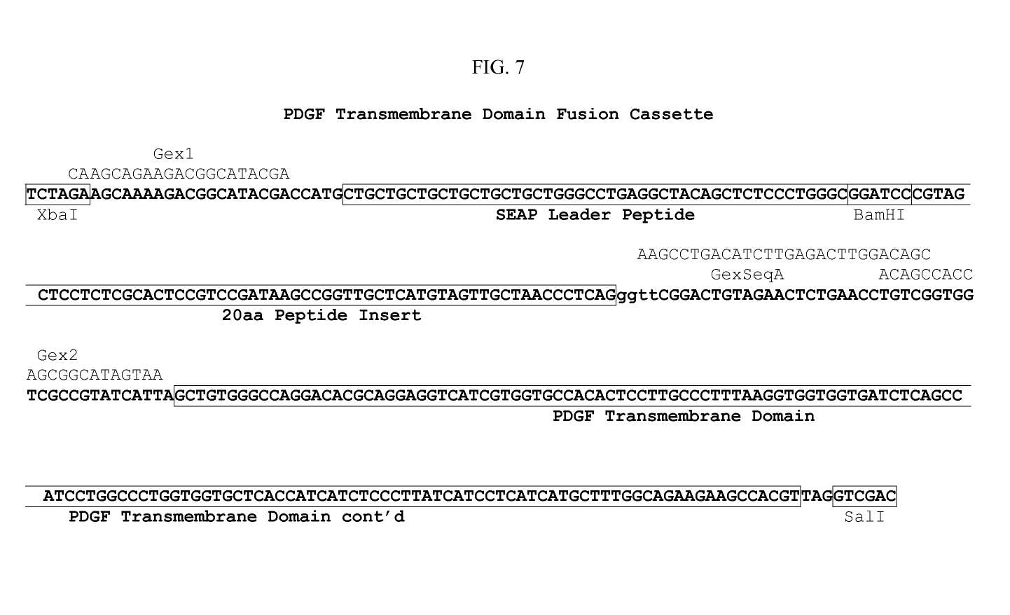

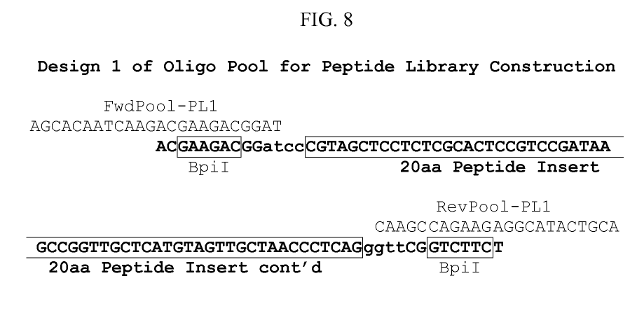

Linear peptides are prone to proteolysis and often possess low biological activity due to their conformational flexibility (Hosse et al., 2006, Protein Sci. 15: 14-27; Skerra, 2007, Curr. Opin. Biotech. 18: 295-304; Binz et al., 2005, Nature Biotechnol. 23: 1257-68). Constrained cyclic peptide libraries resistant to proteolysis are provided by introducing nucleic acid sequences encoding dimerization sequences (EFLIVKS; SEQ ID NO: 45) (see, e.g., FIGS. 1 and 6) flanking the peptide-encoding inserts (Lorens et al., 2000). In alternative embodiments, constructs are provided wherein the secreted peptides are fused to the transmembrane domain of PDGF (see, e.g., FIGS. 1 and 7). The rationale for the transmembrane embodiments of the invention is that peptide-transmembrane PDGF fusion constructs can activate receptors more effectively due to the increase of local concentrations of peptides on the cell surface, and reduce the “bystander effect” by lowering the concentration of free peptides in solution. In other embodiments, the invention provides recombinant expression constructs wherein the peptide inserts are fused to antibody Fc domain (Baud and Karlin, 1999; Yang and Honig, 2000; Koch and Waldmann, 2005) or albumin (Zhang et al., 2003, Biochem. Biophys. Res. Comm. 310: 1181-87), in order to explore the functional activity of peptide modulators in the carrier protein constructs, which have previously been successfully used for the development of biologics with high efficacy and stability in serum.

In other embodiments, the invention provides a reading-frame selection lentiviral vector (Lutz et al., 2002, Prot. Engineer. 15: 1025-30). In such embodiments, the reading-frame peptide expression vector will comprise an internal CMV-Tet promoter for co-expression of the peptide cassette and a drug resistance (puro) or reporter (renilla fluorescent protein, RFP) gene separated by a self-cleavable 2A peptide (Felp et al., 2006, FRENDS Biotech. 24: 68-75). The use of puromycin as a selection marker (or RFP) in these vectors provides the capacity to exploit enrichment of transduced cells that express the correct peptide cassettes (i.e., without a frame shift).

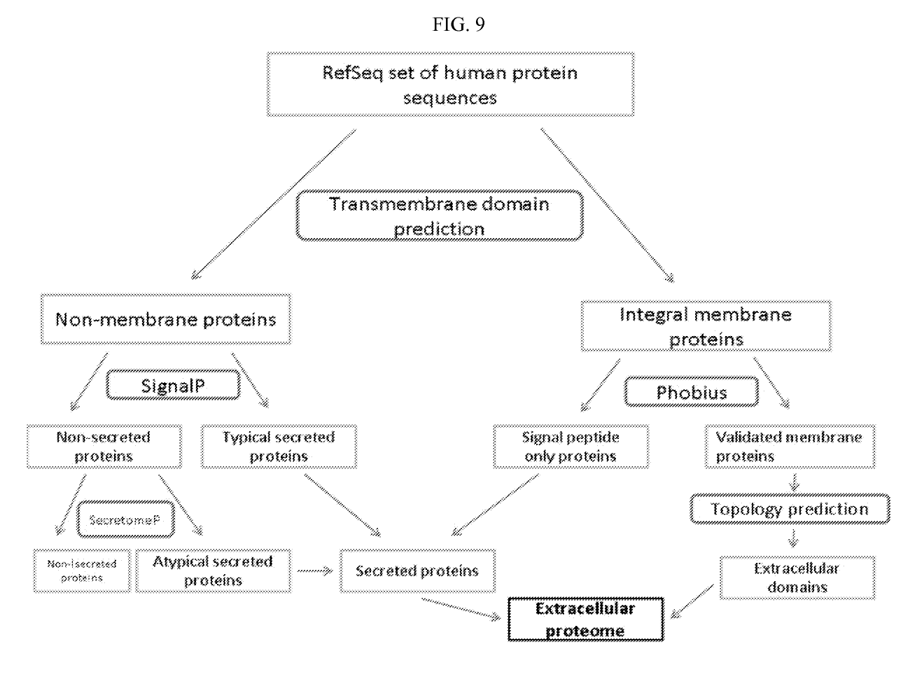

The invention provides a plurality of recombinant expression constructs as described herein encoding peptides derived from the eukaryotic, particularly the mammalian and specifically the human, extracellular proteome. In order to delineate a robust, comprehensive set of human extracellular proteins and domains, protein topology prediction methods are combined in a customized pipeline as shown in FIG. 9. This pipeline also includes annotation of the predicted extracellular protein moieties for functional domains and experimentally characterized functions that are required for analysis and evaluation of the experimental results. The pipeline can be implemented to function in a semiautomatic regime using custom PERL scripts to run all the incorporated software tools and integrate the results.

The peptide delineation protocol begins with a prediction of transmembrane regions for the entire reference set of human proteins. To ensure that the prediction is both robust and as complete as possible, multiple predictive methods are applied and only those putative transmembrane regions that are consistently predicted by at least two methods are scored as positive. The following software tools can be applied for transmembrane region prediction: PredictProtein (Rost et al., 1995, Protein Sci. 4: 521-33; Rost, 1996, Meth. Enzymol. 266: 424-539), TMAP (Persson and Argos, 1997, J. Prot. Chem. 16: 453-57), TMHMM (Kali et al., 2004, J. Mol. Biol. 338: 1027-36), and TMPRED (Hoffmann and Stoffel, 1993, Biol. Chem. 347: 166)—as generally recommended for reliable transmembrane region prediction (Bigelow and Rost, 2009, Methods Mol. Biol. 528: 3-23). All software is executed automatically on the entire set of validated human proteins from the NCBI RefSeq database. Those proteins for which at least two methods predict at least one transmembrane segment with an overlap of at least 15 amino acid residues are classified as “integral membrane” proteins and the remaining proteins classified as “non-membrane.”

The great majority of soluble, extracellular proteins possess N-terminal signal peptides.

Signal peptides can be predicted in the set of non-membrane proteins using the SignalP program (Bendtsen et al., 2004, J. Mol. Biol. 340: 783-95; Emanuelsson et al., 2007, Nat. Protoc. 2: 953-71), and the proteins for which signal peptides are predicted are classified as “typical secreted.” The remaining non-membrane proteins can be analyzed for the presence of non-canonical secretion signals using the SecretomeP program (Bendtsen et al., 2004, Protein Eng. Des. Sci.

17: 349-56), and those proteins for which such signals are predicted are classified as “atypical secreted.” For the “integral membrane” proteins, Phobius software (Kali et al., 2007, Nucl. Acids Res. 35: W429-32) can be used to identify signal peptides erroneously predicted as transmembrane regions, and the proteins containing signal peptides only are moved to the secreted protein set. For the remaining predicted integral membrane proteins, membrane topology can be predicted using the HMMTOP (Tusnady and Simon, 2001, Bioinformatics 17: 849-50) and PredictProtein (Rost et al., 1996, Protein Sci. 5: 1704-14) methods, and the extracellular regions consistently predicted by both methods to exceed 20 amino acid residues in length can be extracted from each protein sequence using a custom script.

The set of secreted proteins and extracellular domains of membrane proteins (estimated approximately 2,000) predicted as described herein are annotated for the presence of known functional domains using the Conserved Domain Database (CDD) at the NCBI (Marchler-Bauer et al., 2009). In addition, the annotation from the GenBank database can be extracted and linked to each sequence in a customized database. The developed set of the predicted proteins can be validated against a list of known extracellular and membrane proteins, including well-characterized sets of human cytokines, chemokines, growth factors and receptors. At least 90% overlap between predicted and known sets of secreted and membrane proteins can be expected. If the overlap is less than 90%, prediction tools can be further optimized and the protein database amended to include with protein candidates selected from NCBI RefSeq and the Entrez Protein Database using MeSH term key word search for, inter alia, cytokine, chemokine, growth factor, receptor (extracellular domains), cell surface, extracellular, and cell-cell communication. One embodiment of a portion of the human extracellular proteome used for preparing libraries of peptide-encoding recombinant expression constructs as set forth herein is shown in Table 1.

TABLE 1

| | | GenBank |
|---|---|---|
| Abbreviation | Name | Accession No. |
| V3 | | |
| A1BG | alpha-1-B glycoprotein | BC035719 |
| ACE | angiotensin I converting enzyme (peptidyl-dipeptidase A) 1 | BC036375 |
| ACE2 | angiotensin I converting enzyme (peptidyl-dipeptidase A) 2 | BC048094 |
| ACHE | acetylcholinesterase (Yt blood group) | BC143469 |
| ADAMTS4 | ADAM metallopeptidase with thrombospondin type 1 motif, 4 | BC063293 |
| ADAMTS5 | ADAM metallopeptidase with thrombospondin type 1 motif, 5 | BC093777 |
| ADCYAP1 | adenylate cyclase activating polypeptide 1 (pituitary) | BC101803 |
| ADFP | adipose differentiation-related protein | BC005127 |
| ADIPOQ | adiponectin, C1Q and collagen domain containing | BC096308 |
| ADM | adrenomedullin | BC015961 |
| AFM | afamin | BC109020 |
| AGGF1 | angiogenic factor with G patch and FHA domains 1 | BC032844 |
| AGRP | agouti related protein homolog (mouse) | BC110443 |
| AGT | angiotensinogen (serpin peptidase inhibitor, clade A, member 8) | BC011519 |
| AHSG | alpha-2-HS-glycoprotein | BC052590 |
| AKR1B1 | aldo-keto reductase family 1, member B1 (aldose reductase) | BC010391 |
| ALB | albumin | BC034023 |
| AMBN | ameloblastin (enamel matrix protein) | BC106932 |
| AMBP | alpha-1-microglobulin/bikunin precursor | BC041593 |
| AMELX | amelogenin (amelogenesis imperfecta 1, X-linked) | BC074951 |
| AMH | anti-Mullerian hormone | BC049194 |
| AMP18 | | |
| AMTN | amelotin | BC121817 |
| AMY2A | amylase, alpha 2A (pancreatic) | BC146997 |
| ANG | angiogenin, ribonuclease, RNase A family, 5 | BC020704 |
| ANGPT1 | angiopoietin 1 | BC152419 |
| ANGPT2 | angiopoietin 2 | BC143902 |
| ANGPT4 | angiopoietin 4 | BC111978 |
| ANGPTL1 | angiopoietin-like 1 | BC050640 |
| ANGPTL3 | angiopoietin-like 3 | BC058287 |
| ANGPTL4 | angiopoietin-like 4 | BC023647 |
| APCS | amyloid P component, serum | BC007058 |
| APLP1 | amyloid beta (A4) precursor-like protein 1 | BC012889 |
| APOA1 | apolipoprotein A-I | BC110286 |
| APOA1BP | apolipoprotein A-I binding protein | BC100934 |
| APOA2 | apolipoprotein A-II | BC005282 |
| APOA4 | apolipoprotein A-IV | BC074764 |
| APOA5 | apolipoprotein A-V | BC101789 |
| APOC2 | apolipoprotein C-II | BC005348 |
| APOC3 | apolipoprotein C-III | BC134419 |
| APOD | apolipoprotein D | BC007402 |
| APOE | apolipoprotein E | BC003557 |
| APOF | apolipoprotein F | BC026257 |
| APOH | apolipoprotein H (beta-2-glycoprotein I) | BC026283 |
| APOL1 | apolipoprotein L, 1 | BC143039 |
| APP | amyloid beta (A4) precursor protein | BC065529 |
| AREG | amphiregulin | BC146967 |
| ARP2 | activation-induced cytidine deaminase | BC006296 |
| ARTN | artemin | BC062375 |
| ATG4C | ATG4 autophagy related 4 homolog C (S. cerevisiae) | BC033024 |
| AZGP1 | alpha-2-glycoprotein 1, zinc-binding | BC033830 |
| AZU1 | azurocidin 1 | BC093933 |
| B7-H3 | CD276 molecule | BC062581 |
| B7H2 | inducible T-cell co-stimulator ligand | BC064637 |
| BCHE | butyrylcholinesterase | BC018141 |
| BDNF | brain-derived neurotrophic factor | BC029795 |
| BGLAP | bone gamma-carboxyglutamate (gla) protein | BC113434 |
| BGN | biglycan | BC002416 |
| BMP1 | bone morphogenetic protein 1 | BC136679 |
| BMP2 | bone morphogenetic protein 2 | BC140325 |
| BMP3 | bone morphogenetic protein 3 | BC117514 |
| BMP4 | bone morphogenetic protein 4 | BC020546 |
| BMP5 | bone morphogenetic protein 5 | BC027958 |
| BMP6 | bone morphogenetic protein 6 | BC160106 |
| BMP8 | bone morphogenetic protein 8b (BMP8B) | NM_001720 |
| BMP15 | bone morphogenetic protein 15 | BC069155 |
| BPIL2 | bactericidal/permeability-increasing protein-like 2 | BC131582 |
| BRE | brain and reproductive organ-expressed (TNFRSF1A modulator) | BC001251 |
| BTC | betacellulin | BC011618 |
| C19orf2 | chromosome 19 open reading frame 2 | BC067259 |
| C1QA | complement component 1, q subcomponent, A chain | BC071986 |
| C1QB | complement component 1, q subcomponent, B chain | BC008983 |
| C1QC | complement component 1, q subcomponent, C chain | BC009016 |
| C1QTNF3 | C1q and tumor necrosis factor related protein 3 | BC112925 |
| C1R | complement component 1, r subcomponent | BC035220 |
| C1S | complement component 1, s subcomponent | BC056903 |
| C2 | complement component 2 | BC043484 |
| C20orf1 | | |
| C20orf9 | | |
| C4BPA | complement component 4 binding protein, alpha | BC022312 |
| C4BPB | complement component 4 binding protein, beta | BC005378 |
| C6 | complement component 6 | BC035723 |
| C7 | complement component 7 | BC063851 |
| C8A | complement component 8, alpha polypeptide | BC132913 |
| C8B | complement component 8, beta polypeptide | BC130575 |
| C8G | complement component 8, gamma polypeptide | BC113626 |
| CABP4 | calcium binding protein 4 | BC033167 |
| CALCB | calcitonin-related polypeptide beta | BC092468 |
| CARTPT | CART prepropeptide | BC029882 |
| CCK | cholecystokinin | BC093055 |
| CCL1 | chemokine (C-C motif) ligand 1 | BC105075 |
| CCL2 | chemokine (C-C motif) ligand 2 | BC009716 |
| CCL3 | chemokine (C-C motif) ligand 3 | BC171831 |
| CCL3L1 | chemokine (C-C motif) ligand 3-like 1 | BC107710 |
| CCL3L3 | chemokine (C-C motif) ligand 3-like 3 | BC146914 |
| CCL4 | chemokine (C-C motif) ligand 4 | BC104227 |
| CCL4L1 | chemokine (C-C motif) ligand 4-like 1 | BC144394 |
| CCL5 | chemokine (C-C motif) ligand 5 | BC008600 |
| CCL7 | chemokine (C-C motif) ligand 7 | BC092436 |
| CCL8 | chemokine (C-C motif) ligand 8 | BC126242 |
| CCL11 | chemokine (C-C motif) ligand 11 | BC017850 |
| CCL13 | chemokine (C-C motif) ligand 13 | BC008621 |
| CCL14 | chemokine (C-C motif) ligand 14 | BC045165 |
| CCL15 | chemokine (C-C motif) ligand 15 | BC140941 |
| CCL16 | chemokine (C-C motif) ligand 16 | BC099662 |
| CCL17 | chemokine (C-C motif) ligand 17 | BC069107 |
| CCL18 | chemokine (C-C motif) ligand 18 (pulmonary and activation- | BC096125 |
| | regulated) | |
| CCL19 | chemokine (C-C motif) ligand 19 | BC027968 |
| CCL20 | chemokine (C-C motif) ligand 20 | BC020698 |
| CCL21 | chemokine (C-C motif) ligand 21 | BC027918 |
| CCL22 | chemokine (C-C motif) ligand 22 | BC027952 |
| CCL23 | chemokine (C-C motif) ligand 23 | BC143310 |
| CCL24 | chemokine (C-C motif) ligand 24 | BC069391 |
| CCL25 | chemokine (C-C motif) ligand 25 | BC144463 |
| CCL26 | chemokine (C-C motif) ligand 26 | BC101665 |
| CCL27 | chemokine (C-C motif) ligand 27 | BC148263 |
| CCL28 | chemokine (C-C motif) ligand 28 | BC062668 |
| CD14 | CD14 molecule | BC010507 |
| CD248 | CD248 molecule, endosialin | BC051340 |
| CD27 | CD27 molecule | BC012160 |
| CD40LG | CD40 ligand | BC074950 |
| CD5L | CD5 molecule-like | BC033586 |
| CD86 | CD86 molecule | BC040261 |
| CDA | cytidine deaminase | BC054036 |
| CDH13 | cadherin 13, H-cadherin (heart) | BC030653 |
| CEACAM8 | carcinoembryonic antigen-related cell adhesion molecule 8 | BC026263 |
| CECR1 | cat eye syndrome chromosome region, candidate 1 | BC051755 |
| CEL | carboxyl ester lipase (bile salt-stimulated lipase) | BC042510 |
| CER1 | cerberus 1, cysteine knot superfamily, homolog (Xenopus laevis) | BC103976 |
| CETP | cholesteryl ester transfer protein, plasma | BC025739 |
| CFB | complement factor B | BC007990 |
| CFD | complement factor D (adipsin) | BC057807 |
| CFHR1 | complement factor H-related 1 | BC107771 |
| CFHR3 | complement factor H-related 3 | BC058009 |
| CFHR5 | complement factor H-related 5 | BC111773 |
| CFP | complement factor properdin | BC015756 |
| CGA | glycoprotein hormones, alpha polypeptide | BC055080 |
| CGB | chorionic gonadotropin, beta polypeptide | BC128603 |
| CGB5 | chorionic gonadotropin, beta polypeptide 5 | BC106724 |
| CGB7 | chorionic gonadotropin, beta polypeptide 7 | BC160150 |
| CGB8 | chorionic gonadotropin, beta polypeptide 8 | BC103969 |
| CHAD | chondroadherin | BC073974 |
| CHGB | chromogranin B (secretogranin 1) | BC000375 |
| CHI3L1 | chitinase 3-like 1 (cartilage glycoprotein-39) | BC039132 |
| CHI3L2 | chitinase 3-like 2 | BC011460 |
| CHIA | chitinase, acidic | BC106910 |
| CHIT1 | chitinase 1 (chitotriosidase) | BC105681 |
| CHRDL1 | chordin-like 1 | BC002909 |
| CKLF | chemokine-like factor | BC091478 |
| CKLFSF2 | chemokine-like factor super family member 2 | AF479260 |
| CKLFSF3 | chemokine-like factor super family member 3 | AF479813 |
| CKLFSF4 | chemokine-like factor super family member 4 | AF521889 |
| CKLFSF5 | chemokine-like factor super family member 5 | AF479262 |
| CKLFSF6 | chemokine-like factor super family member 6 | AF479261 |
| CKLFSF7 | chemokine-like factor super family member 7 | AF479263 |
| CKLFSF8 | chemokine-like factor super family member 8 | AF474370 |
| CLC | Charcot-Leyden crystal protein | BC119711 |
| CLCA3 | chloride channel, calcium activated, family member 3 | AL356270 |
| CLCF1 | cardiotrophin-like cytokine factor 1 | BC066229 |
| CLEC11A | C-type lectin domain family 11 | BC005810 |
| CLEC3B | C-type lectin domain family 3, member B | BC011024 |
| CLU | clusterin | BC019588 |
| CNP | 2′,3′-cyclic nucleotide 3′ phosphodiesterase | BC011046 |
| CNTF | ciliary neurotrophic factor | BC074964 |
| COL6A2 | collagen, type VI, alpha 2 | BC065509 |
| COL8A1 | collagen, type VIII, alpha 1 | BC013581 |
| COL8A2 | collagen, type VIII, alpha 2 | BC096296 |
| COL9A1 | collagen, type IX, alpha 1 | BC063646 |
| COL9A2 | collagen, type IX, alpha 2 | BC136326 |
| COL9A3 | collagen, type IX, alpha 3 | BC011705 |
| COL10A1 | collagen, type X, alpha 1 | BC130623 |
| COL13A1 | collagen, type XIII, alpha 1 | BC136385 |
| COL25A1 | collagen, type XXV, alpha 1 | BC036669 |
| COLQ | collagen-like tail subunit (single strand of homotrimer) of | BC074828 |
| | asymmetric acetylcholinesterase | |
| COMP | cartilage oligomeric matrix protein | BC125092 |
| CORT | cortistatin | BC119724 |
| CPA1 | carboxypeptidase A1 (pancreatic) | BC005279 |
| CPB2 | carboxypeptidase B2 (plasma) | BC007057 |
| CPN1 | carboxypeptidase N, polypeptide 1 | BC027897 |
| CPN2 | carboxypeptidase N, polypeptide 2 | BC137403 |
| CRH | corticotropin releasing hormone | BC002599 |
| CRISP1 | cysteine-rich secretory protein 1 | BC160072 |
| CRISP2 | cysteine-rich secretory protein 2 | BC022011 |
| CRISP3 | cysteine-rich secretory protein 3 | BC101539 |
| CRLF1 | cytokine receptor-like factor 1 | BC044634 |
| CRP | C-reactive protein, pentraxin-related | BC125135 |
| CSF1 | colony stimulating factor 1 (macrophage) | BC021117 |
| CSF2 | colony stimulating factor 2 (granulocyte-macrophage) | BC108724 |
| CSF3 | colony stimulating factor 3 (granulocyte) | BC033245 |
| CSH1 | chorionic somatomammotropin hormone 1 (placental lactogen) | BC057768 |
| CSH2 | chorionic somatomammotropin hormone 2 | BC119748 |
| CSHL1 | chorionic somatomammotropin hormone-like 1 | BC119747 |
| CSN3 | casein kappa | BC010935 |
| CSPG5 | CSPG5 protein | BC111583 |
| CTF1 | cardiotrophin 1 | BC064416 |
| CTGF | connective tissue growth factor | BC087839 |
| CTRB1 | chymotrypsinogen B1 | BC005385 |
| CTRL | chymotrypsin-like | BC063475 |
| CTSD | cathepsin D | BC016320 |
| CTSL1 | cathepsin L1 | BC142983 |
| CTSS | cathepsin S | BC002642 |
| CX3CL1 | chemokine (C-X3-C motif) ligand 1 | BC016164 |
| CXCL1 | chemokine (C—X—C motif) ligand 1 (melanoma growth stimulating | BC011976 |
| | activity, alpha) | |
| CXCL2 | chemokine (C—X—C motif) ligand 2 | BC015753 |
| CXCL3 | chemokine (C—X—C motif) ligand 3 | BC065743 |
| CXCL5 | chemokine (C—X—C motif) ligand 5 | BC008376 |
| CXCL6 | chemokine (C—X—C motif) ligand 6 (granulocyte chemotactic | BC013744 |
| | protein 2) | |
| CXCL9 | chemokine (C—X—C motif) ligand 9 | BC095396 |
| CXCL10 | chemokine (C—X—C motif) ligand 10 | BC010954 |
| CXCL11 | chemokine (C—X—C motif) ligand 11 | BC110986 |
| CXCL12 | chemokine (C—X—C motif) ligand 12 (stromal cell-derived factor 1) | BC039893 |
| CXCL13 | chemokine (C—X—C motif) ligand 13 | BC012589 |
| CXCL14 | chemokine (C—X—C motif) ligand 14 | BC003513 |
| CXCL16 | chemokine (C—X—C motif) ligand 16 | BC044930 |
| CYR61 | cysteine-rich, angiogenic inducer, 61 | BC009199 |
| CYTL1 | cytokine-like 1 | BC031391 |
| DBH | dopamine beta-hydroxylase (dopamine beta-monooxygenase) | BC017174 |
| DCD | dermcidin | BC069108 |
| DEFB103 | defensin, beta 103A | NM_018661 |
| DEFB106 | beta-defensin (DEFB106) | AF529417 |
| DGCR6 | DiGeorge syndrome critical region gene 6 | BC047039 |
| DKK1 | dickkopf homolog 1 (Xenopus laevis) | BC001539 |
| DKK2 | dickkopf homolog 2 (Xenopus laevis) | BC126330 |
| DKK3 | dickkopf homolog 3 (Xenopus laevis) | BC007660 |
| DKKL1 | dickkopf-like 1 (soggy) | BC030581 |
| DLK1 | delta-like 1 homolog (Drosophila) | BC007741 |
| DLL1 | delta-like 1 (Drosophila) | BC152803 |
| DLL3 | delta-like 3 (Drosophila) | BC000218 |
| DLL4 | delta-like 4 (Drosophila) | BC106950 |
| DMP1 | dentin matrix acidic phosphoprotein 1 | BC132865 |
| DNASE1 | deoxyribonuclease I | BC029437 |
| EBI3 | Epstein-Barr virus induced 3 | BC046112 |
| ECM1 | extracellular matrix protein 1 | BC023505 |
| ECM2 | extracellular matrix protein 2, female organ and adipocyte specific | BC107493 |
| EDN1 | endothelin 1 | BC009720 |
| EDN2 | endothelin 2 | BC034393 |
| EDN3 | endothelin 3 | BC008876 |
| EFEMP1 | EGF-containing fibulin-like extracellular matrix protein 1 | BC098561 |
| EFEMP2 | EGF-containing fibulin-like extracellular matrix protein 2 | BC010456 |
| EFNA1 | ephrin-A1 | BC095432 |
| EFNA2 | ephrin-A2 | BC146278 |
| EFNA3 | ephrin-A3 | BC110406 |
| EFNA4 | ephrin-A4 | BC107483 |
| EFNA5 | ephrin-A5 | BC075054 |
| EFNB1 | ephrin-B1 | BC052979 |
| EFNB2 | ephrin-B2 | BC105956 |
| EGFL6 | EGF-like-domain, multiple 6 | BC038587 |
| EGFL7 | EGF-like-domain, multiple 7 | BC088371 |
| ELA2 | elastase 2, neutrophil | BC074817 |
| ELA2B | elastase 2B | BC069412 |
| ELA3B | elastase 3B, pancreatic | BC005216 |
| ELN | elastin | BC065566 |
| ENPP1 | ectonucleotide pyrophosphatase/phosphodiesterase 1 | BC059375 |
| ENSA | endosulfine alpha | BC069208 |
| EPGN | epithelial mitogen homolog (mouse) | BC127938 |
| EPO | erythropoietin | BC143225 |
| ERAP1 | endoplasmic reticulum aminopeptidase 1 | BC030775 |
| EREG | epiregulin | BC136404 |
| ESDN | discoidin, CUB and LCCL domain containing 2 | BC029658 |
| ESM1 | endothelial cell-specific molecule 1 | BC011989 |
| F2 | coagulation factor II (thrombin) | BC051332 |
| F3 | coagulation factor III (thromboplastin, tissue factor) | BC011029 |
| F7 | coagulation factor VII (serum prothrombin conversion accelerator) | BC130468 |
| F8A | coagulation factor VIII, procoagulant component | BC166700 |
| F9 | coagulation factor IX | BC109214 |
| F10 | coagulation factor X | BC046125 |
| F11 | coagulation factor XI | BC122863 |
| F12 | coagulation factor XII (Hageman factor) | BC168381 |
| F13A1 | coagulation factor XIII, A1 polypeptide | BC027963 |
| F13B | coagulation factor XIII, B polypeptide | BC148333 |
| FAM12A | family with sequence similarity 12, member A | BC106712 |
| FAM12B | family with sequence similarity 12, member B (epididymal) | BC128030 |
| FAM3B | family with sequence similarity 3, member B | BC057829 |
| FAM3C | family with sequence similarity 3, member C | BC068526 |
| FAM3D | family with sequence similarity 3, member D | BC015359 |
| FASLG | Fas ligand (TNF superfamily, member 6) | BC017502 |
| FBLN1 | fibulin 1 | BC022497 |
| FBLN5 | fibulin 5 | BC022280 |
| FBS1 | F-box protein 2 | BC096747 |
| FCN3 | ficolin (collagen/fibrinogen domain containing) 3 (Hakata antigen) | BC020731 |
| FETUB | fetuin B | BC074734 |
| FGA | fibrinogen alpha chain | BC101935 |
| FGB | fibrinogen beta chain | BC106760 |
| FGF1 | fibroblast growth factor 1 (acidic) | BC032697 |
| FGF2 | fibroblast growth factor 2 | BC166646 |
| FGF3 | fibroblast growth factor 3 (murine mammary tumor virus | BC113739 |
| | integration site (v-int-2) oncogene homolog) | |
| FGF4 | fibroblast growth factor 4 | BC172495 |
| FGF5 | fibroblast growth factor 5 | BC131502 |
| FGF6 | fibroblast growth factor 6 | BC121098 |
| FGF7 | fibroblast growth factor 7 | BC010956 |
| FGF8 | fibroblast growth factor 8 (androgen-induced) | BC128235 |
| FGF9 | fibroblast growth factor 9 (glia-activating factor) | BC103979 |
| FGF10 | fibroblast growth factor 10 | BC105021 |
| FGF11 | fibroblast growth factor 11 | BC108265 |
| FGF12 | fibroblast growth factor 12 | BC022524 |
| FGF13 | fibroblast growth factor 13 | BC034340 |
| FGF14 | fibroblast growth factor 14 | BC100922 |
| FGF16 | fibroblast growth factor 16 | BC148639 |
| FGF17 | fibroblast growth factor 17 | BC143789 |
| FGF18 | fibroblast growth factor 18 | BC006245 |
| FGF19 | fibroblast growth factor 19 | BC017664 |
| FGF20 | fibroblast growth factor 20 | BC137447 |
| FGF21 | fibroblast growth factor 21 | BC018404 |
| FGF22 | fibroblast growth factor 22 | BC137445 |
| FGF23 | fibroblast growth factor 23 | BC096713 |
| FGFBP1 | fibroblast growth factor binding protein 1 | BC003628 |
| FGG | fibrinogen gamma chain | BC021674 |
| FGL1 | fibrinogen-like 1 | BC007047 |
| FGL2 | fibrinogen-like 2 | BC073986 |
| FIGF | c-fos induced growth factor (vascular endothelial growth factor D) | BC027948 |
| FKTN | fukutin | BC117700 |
| FLJ2113 | | |
| FLRT1 | fibronectin leucine rich transmembrane protein 1 | BC018370 |
| FLRT2 | fibronectin leucine rich transmembrane protein 2 | BC143936 |
| FLRT3 | fibronectin leucine rich transmembrane protein 3 | BC020870 |
| FLT3LG | fms-related tyrosine kinase 3 ligand | BC136464 |
| FMOD | fibromodulin | BC035281 |
| FN1 | fibronectin 1 | BC143763 |
| FRZB | frizzled-related protein | BC027855 |
| FSHB | follicle stimulating hormone, beta polypeptide | BC113490 |
| FST | follistatin | BC004107 |
| FSTL1 | follistatin-like 1 | BC000055 |
| FSTL3 | follistatin-like 3 (secreted glycoprotein) | BC005839 |
| FURIN | furin (paired basic amino acid cleaving enzyme) | BC012181 |
| FXYD6 | FXYD domain containing ion transport regulator 6 | BC093040 |
| GAL | galanin prepropeptide | BC030241 |
| GALP | galanin-like peptide | BC141468 |
| GAS | | |
| GC | group-specific component (vitamin D binding protein) | BC057228 |
| GCG | glucagon | BC005278 |
| GDF1 | growth differentiation factor 1 | BC022450 |
| GDF2 | growth differentiation factor 2 | BC074921 |
| GDF3 | growth differentiation factor 3 | BC030959 |
| GDF5 | growth differentiation factor 5 | BC032495 |
| GDF7 | growth differentiation factor 7 | BC160118 |
| GDF9 | growth differentiation factor 9 | BC096230 |
| GDF10 | growth differentiation factor 10 | BC028237 |
| GDF11 | growth differentiation factor 11 | BC148591 |
| GDF15 | growth differentiation factor 15 | BC000529 |
| GDNF | glial cell derived neurotrophic factor | BC128108 |
| GFER | growth factor, augmenter of liver regeneration | BC002429 |
| GH1 | growth hormone 1 | BC090045 |
| GH2 | growth hormone 2 | BC020760 |
| GHRH | growth hormone releasing hormone | BC099727 |
| GHRL | ghrelin/obestatin prepropeptide | BC025791 |
| GIP | gastric inhibitory polypeptide | BC096148 |
| GLA | galactosidase, alpha | BC002689 |
| GLMN | glomulin, FKBP associated protein | BC001257 |
| GMFB | glia maturation factor, beta | BC005359 |
| GMFG | glia maturation factor, gamma | BC143548 |
| GNAS | GNAS complex locus | BC108315 |
| GNG8 | guanine nucleotide binding protein (G protein), gamma 8 | BC095514 |
| GNGT2 | guanine nucleotide binding protein (G protein), gamma | BC008663 |
| | transducing activity polypeptide 2 | |
| GNL1 | guanine nucleotide binding protein-like 1 | BC013959 |
| GNLY | granulysin | BC023576 |
| GNRH1 | gonadotropin-releasing hormone 1 (luteinizing-releasing hormone) | BC126437 |
| GNRH2 | gonadotropin-releasing hormone 2 | BC115400 |
| GPB5 | glycoprotein hormone beta 5 | BC069113 |
| GPC1 | glypican 1 | BC051279 |
| GPHA2 | glycoprotein hormone alpha 2 | BC101523 |
| GPI | glucose phosphate isomerase | BC004982 |
| GPX3 | glutathione peroxidase 3 (plasma) | BC050378 |
| GREM1 | gremlin 1, cysteine knot superfamily, homolog (Xenopus laevis) | BC101611 |
| GREM2 | gremlin 2, cysteine knot superfamily, homolog (Xenopus laevis) | BC046632 |
| GRN | granulin | BC000324 |
| GRP | galectin-related protein | BC062691 |
| GSN | gelsolin (amyloidosis, Finnish type) | BC026033 |
| GUCA2A | guanylate cyclase activator 2A (guanylin) | BC140428 |
| GUCA2B | guanylate cyclase activator 2B (uroguanylin) | BC093781 |
| HABP2 | hyaluronan binding protein 2 | BC031412 |
| HAMP | hepcidin antimicrobial peptide | BC020612 |
| HAPLN1 | hyaluronan and proteoglycan link protein 1 | BC057808 |
| HBEGF | heparin-binding EGF-like growth factor | BC033097 |
| HCRT | HCRT protein | BC111915 |
| HDGF | hepatoma-derived growth factor (high-mobility group protein 1- | BC018991 |
| | like) | |
| HGFAC | HGF activator | BC112190 |
| HMOX1 | heme oxygenase (decycling) 1 | BC001491 |
| HPX | hemopexin | BC005395 |
| HRG | histidine-rich glycoprotein | BC150591 |
| HS3ST4 | heparan sulfate (glucosamine) 3-O-sulfotransferase 4 | BC156387 |
| HTN1 | histatin 1 | BC017835 |
| HTN3 | histatin 3 | BC095438 |
| HTRA1 | HtrA serine peptidase 1 | BC172536 |
| HYAL1 | hyaluronoglucosaminidase 1 | BC035695 |
| IAPP | islet amyloid polypeptide precursor | DQ516082 |
| ICAM1 | intercellular adhesion molecule 1 | BC015969 |
| IDE | insulin-degrading enzyme | BC096339 |
| IFI30 | interferon, gamma-inducible protein 30 | BC031020 |
| IFNA1 | interferon, alpha 1 | BC112302 |
| IFNA2 | interferon, alpha 2 | BC104164 |
| IFNA4 | interferon, alpha 4 | BC113640 |
| IFNA5 | interferon, alpha 5 | BC093757 |
| IFNA6 | interferon, alpha 6 | BC098357 |
| IFNA7 | interferon, alpha 7 | BC074991 |
| IFNA8 | interferon, alpha 8 | BC104830 |
| IFNA10 | interferon, alpha 10 | BC103972 |
| IFNA13 | interferon, alpha 13 | BC093988 |
| IFNA14 | interferon, alpha 14 | BC104159 |
| IFNA16 | interferon, alpha 16 | BC140290 |
| IFNA17 | interferon, alpha 17 | BC098355 |
| IFNA21 | interferon, alpha 21 | BC101638 |
| IFNAR2 | interferon (alpha, beta and omega) receptor 2 | BC002793 |
| IFNB1 | interferon, beta 1, fibroblast | BC096150 |
| IFNG | interferon, gamma | BC070256 |
| IFNK | interferon, kappa | BC140280 |
| IFNT1 | interferon, epsilon | BC100872 |
| IFNW1 | interferon, omega 1 | BC069095 |
| IFRD1 | interferon-related developmental regulator 1 | BC001272 |
| IGF2 | insulin-like growth factor 2 (somatomedin A) | BC000531 |
| IGFALS | insulin-like growth factor binding protein, acid labile subunit | BC025681 |
| IGFBP1 | insulin-like growth factor binding protein 1 | BC035263 |
| IGFBP3 | insulin-like growth factor binding protein 3 | BC064987 |
| IGFBP5 | insulin-like growth factor binding protein 5 | BC011453 |
| IGJ | immunoglobulin J polypeptide, linker protein for immunoglobulin | BC038982 |
| | alpha and mu polypeptides | |
| IHH | Indian hedgehog homolog (Drosophila) | BC136588 |
| IK | IK cytokine, down-regulator of HLA II | BC071964 |
| IL1A | interleukin 1, alpha | BC013142 |
| IL1B | interleukin 1, beta | BC008678 |
| IL1F5 | interleukin 1 family, member 5 (delta) | BC024747 |
| IL1F6 | interleukin 1 family, member 6 (epsilon) | BC107043 |
| IL1F7 | interleukin 1 family, member 7 (zeta) | BC020637 |
| IL1F8 | interleukin 1 family, member 8 (eta) | BC101833 |
| IL1F9 | interleukin 1 family, member 9 | BC098155 |
| IL1RN | interleukin 1 receptor antagonist | BC009745 |
| IL2 | interleukin 2 | BC070338 |
| IL3 | interleukin 3 (colony-stimulating factor, multiple) | BC066275 |
| IL4 | interleukin 4 | BC070123 |
| IL5 | interleukin 5 (colony-stimulating factor, eosinophil) | BC066282 |
| IL5RA | interleukin 5 receptor, alpha | BC027599 |
| IL6 | interleukin 6 (interferon, beta 2) | BC015511 |
| IL6R | interleukin 6 receptor | BC132686 |
| IL7 | interleukin 7 | BC047698 |
| IL8 | interleukin 8 | BC013615 |
| IL9 | interleukin 9 | BC066285 |
| IL9R | interleukin 9 receptor | BC051337 |
| IL10 | interleukin 10 | BC104253 |
| IL11 | interleukin 11 | BC012506 |
| IL12A | interleukin 12A (natural killer cell stimulatory factor 1, cytotoxic | BC104984 |
| | lymphocyte maturation factor 1, p35) | |
| IL12B | interleukin 12B (natural killer cell stimulatory factor 2, cytotoxic | BC074723 |
| | lymphocyte maturation factor 2, p40) | |
| IL13 | interleukin 13 | BC096141 |
| IL13RA2 | interleukin 13 receptor, alpha 2 | BC033705 |
| IL15 | interleukin 15 | BC100962 |
| IL16 | interleukin 16 (lymphocyte chemoattractant factor) | BC136660 |
| IL17A | interleukin 17A | BC067505 |
| IL17B | interleukin 17B | BC113946 |
| IL17C | interleukin 17C | BC069152 |
| IL17D | interleukin 17D | BC036243 |
| IL17E | interleukin 17E | AF461739 |
| IL17F | interleukin 17F | BC070124 |
| IL18 | interleukin 18 (interferon-gamma-inducing factor) | BC007461 |
| IL18BP | interleukin 18 binding protein | BC044215 |
| IL19 | interleukin 19 | BC172584 |
| IL20 | interleukin 20 | BC074949 |
| IL21 | interleukin 21 | BC069124 |
| IL22 | interleukin 22 | BC070261 |
| IL22RA2 | interleukin 22 receptor, alpha 2 | BC125168 |
| IL24 | interleukin 24 | BC009681 |
| IL25 | interleukin 25 | BC104931 |
| IL26 | interleukin 26 | BC066270 |
| IL27 | interleukin 27 | BC062422 |
| IL29 | interleukin 29 (interferon, lambda 1) | BC126183 |
| IL32 | interleukin 32 | BC105602 |
| IMPG1 | interphotoreceptor matrix proteoglycan 1 | BC117450 |
| INHA | inhibin, alpha | BC006391 |
| INHBA | inhibin, beta A | BC007858 |
| INHBB | inhibin, beta B | BC030029 |
| INHBC | inhibin, beta C | BC130326 |
| INHBE | inhibin, beta E | BC005161 |
| INS | insulin | BC005255 |
| INSL3 | insulin-like 3 (Leydig cell) | BC106722 |
| INSL4 | insulin-like 4 (placenta) | BC026254 |
| INSL5 | insulin-like 5 | BC101646 |
| INSL6 | insulin-like 6 | BC126473 |
| INT4 | integrator complex subunit 4 | BC009995 |
| ISG15 | ISG15 ubiquitin-like modifier | BC009507 |
| ITIH1 | inter-alpha (globulin) inhibitor H1 | BC069464 |
| ITIH2 | inter-alpha (globulin) inhibitor H2 | BC132685 |
| ITIH3 | inter-alpha (globulin) inhibitor H3 | BC107605 |
| ITIH4 | inter-alpha (globulin) inhibitor H4 (plasma Kallikrein-sensitive | BC136392 |
| | glycoprotein) | |
| KAL1 | Kallmann syndrome 1 sequence | BC137427 |
| KDSR | 3-ketodihydrosphingosine reductase | BC008797 |
| KERA | keratocan | BC032667 |
| KIRREL3 | kin of IRRE like 3 (Drosophila) | BC101775 |
| KISS1 | KiSS-1 metastasis-suppressor | BC022819 |
| KITLG | KIT ligand | BC143899 |
| KL | klotho | NM_004795 |
| KLK3 | kallikrein-related peptidase 3 | BC056665 |
| KLK4 | kallikrein-related peptidase 4 | BC096177 |
| KLK5 | kallikrein-related peptidase 5 | BC008036 |
| KLK6 | kallikrein-related peptidase 6 | BC015525 |
| KLK8 | kallikrein-related peptidase 8 | BC040887 |
| KLK10 | kallikrein-related peptidase 10 | BC002710 |
| KLK13 | kallikrein-related peptidase 13 | BC069334 |
| KLK14 | kallikrein-related peptidase 14 | BC114614 |
| KLK15 | kallikrein-related peptidase 15 | BC144046 |
| KLKB1 | kallikrein B, plasma (Fletcher factor) 1 | BC117351 |
| KLKL5 | kallikrein-related peptidase 12 | BC136341 |
| KNG1 | kininogen 1 | BC060039 |
| KRTAP1- | | |
| KRTAP5- | | |
| KS1 | zinc finger protein 382 | BC132675 |
| LALBA | lactalbumin, alpha- | BC112318 |
| LAMA4 | laminin, alpha 4 | BC066552 |
| LBP | lipopolysaccharide binding protein | BC022256 |
| LCAT | lecithin-cholesterol acyltransferase | BC014781 |
| LECT2 | leukocyte cell-derived chemotaxis 2 | BC101579 |
| LEFTB | left-right determination factor 1 | BC027883 |
| LEFTY2 | left-right determination factor 2 | BC035718 |
| LEP | leptin | BC069452 |
| LFNG | LFNG O-fucosylpeptide 3-beta-N-acetylglucosaminyltransferase | BC014851 |
| LGALS3 | lectin, galactoside-binding, soluble, 3 | BC068068 |
| LGALS7 | lectin, galactoside-binding, soluble, 7B | BC073743 |
| LGALS8 | lectin, galactoside-binding, soluble, 8 | BC016486 |
| LHB | luteinizing hormone beta polypeptide | BC160107 |
| LIF | leukemia inhibitory factor (cholinergic differentiation factor) | BC093733 |
| LOXL1 | lysyl oxidase-like 1 | BC068542 |
| LOXL2 | lysyl oxidase-like 2 | BC000594 |
| LOXL3 | lysyl oxidase-like 3 | BC071865 |
| LPAL2 | lipoprotein, Lp(a)-like 2 (LPAL2) | BC166644 |
| LPL | lipoprotein lipase | BC011353 |
| LRG1 | leucine-rich alpha-2-glycoprotein 1 | BC070198 |
| LTA | lymphotoxin alpha (TNF superfamily, member 1) | BC034729 |
| LTB | lymphotoxin beta (TNF superfamily, member 3) | BC069330 |
| LUM | lumican | BC035997 |
| LYZ | lysozyme (renal amyloidosis) | BC004147 |
| MAP2K2 | mitogen-activated protein kinase kinase 2 | BC018645 |
| MAPK15 | mitogen-activated protein kinase 15 | BC028034 |
| MASP1 | mannan-binding lectin serine peptidase 1 (C4/C2 activating | BC106946 |
| | component of Ra-reactive factor) | |
| MASP2 | mannan-binding lectin serine peptidase 2 | BC156086 |
| MATN1 | matrilin 1, cartilage matrix protein | BC160064 |
| MATN2 | matrilin 2 | BC010444 |
| MATN3 | matrilin 3 | BC139907 |
| MATN4 | matrilin 4 | BC151219 |
| MBL2 | mannose-binding lectin (protein C) 2, soluble (opsonic defect) | BC096181 |
| MDK | midkine (neurite growth-promoting factor 2) | BC011704 |
| MEP1A | meprin A, alpha (PABA peptide hydrolase) | BC143651 |
| MEP1B | meprin A, beta | BC136559 |
| MEPE | matrix extracellular phosphoglycoprotein | BC128158 |
| MFAP4 | microfibrillar-associated protein 4 | BC062415 |
| MFNG | MFNG O-fucosylpeptide 3-beta-N-acetylglucosaminyltransferase | BC094814 |
| MGP | matrix Gla protein | BC093078 |
| MIA | melanoma inhibitory activity | BC005910 |
| MIF | macrophage migration inhibitory factor (glycosylation-inhibiting | BC053376 |
| | factor) | |
| MLN | motilin | BC112314 |
| MMP2 | matrix metallopeptidase 2 (gelatinase A, 72 kDa gelatinase, 72 kDa | BC002576 |
| | type IV collagenase) | |
| MMP3 | matrix metallopeptidase 3 (stromelysin 1, progelatinase) | BC107490 |
| MMP7 | matrix metallopeptidase 7 (matrilysin, uterine) | BC003635 |
| MMP8 | matrix metallopeptidase 8 (neutrophil collagenase) | BC074988 |
| MMP9 | matrix metallopeptidase 9 (gelatinase B, 92 kDa gelatinase, 92 kDa | BC006093 |
| | type IV collagenase) | |
| MMP10 | matrix metallopeptidase 10 (stromelysin 2) | BC002591 |
| MMP11 | matrix metallopeptidase 11 (stromelysin 3) | BC057788 |
| MMP13 | matrix metallopeptidase 13 (collagenase 3) | BC074808 |
| MMP19 | matrix metallopeptidase 19 | BC050368 |
| MMP20 | matrix metallopeptidase 20 | BC152741 |
| MMP25 | matrix metallopeptidase 25 | BC167800 |
| MMP26 | matrix metallopeptidase 26 | BC101541 |
| MMP28 | matrix metallopeptidase 28 | BC002631 |
| MSLN | mesothelin | BC009272 |
| MSMB | microseminoprotein, beta- | BC005257 |
| MST1 | macrophage stimulating 1 (hepatocyte growth factor-like) | BC048330 |
| MSTN | myostatin | BC074757 |
| MYOC | myocilin, trabecular meshwork inducible glucocorticoid response | BC029261 |
| NAMPT | nicotinamide phosphoribosyltransferase | BC106046 |
| NDP | Norrie disease (pseudoglioma) | NM_000266 |
| NELL2 | NEL-like 2 (chicken) | BC020544 |
| NGF | nerve growth factor (beta polypeptide) | BC126150 |
| NLGN1 | neuroligin 1 | BC032555 |
| NLGN3 | neuroligin 3 | BC051715 |
| NLGN4X | neuroligin 4, X-linked | BC034018 |
| NMB | neuromedin B | BC007407 |
| NMU | neuromedin U | BC012908 |
| NODAL | nodal homolog (mouse) | BC104976 |
| NOG | noggin | BC034027 |
| NPFF | neuropeptide FF-amide peptide precursor | BC104234 |
| NPPA | natriuretic peptide precursor A | BC005893 |
| NPPB | natriuretic peptide precursor B | BC025785 |
| NPPC | natriuretic peptide precursor C | BC105067 |
| NPTX1 | neuronal pentraxin I | BC089441 |
| NPTX2 | neuronal pentraxin II | BC048275 |
| NPY | neuropeptide Y | BC029497 |
| NRG1 | neuregulin 1 | BC150609 |
| NRG2 | neuregulin 2 | BC166615 |
| NRG3 | neuregulin 3 | BC136811 |
| NRTN | neurturin | BC137400 |
| NTF3 | neurotrophin 3 | BC107075 |
| NTF4 | neurotrophin 4 | BC012421 |
| NTN1 | netrin 1 | NM_004822 |
| NTS | neurotensin | BC010918 |
| NUCB1 | nucleobindin 1 | BC002356 |
| NUCB2 | nucleobindin 2 | NM_005013 |
| NUDT6 | nudix (nucleoside diphosphate linked moiety X)-type motif 6 | BC009842 |
| NXPH1 | neurexophilin 1 | BC047505 |
| NXPH2 | neurexophilin 2 | BC104741 |
| NXPH3 | neurexophilin 3 | BC022541 |
| NXPH4 | neurexophilin 4 | BC036679 |
| OGN | osteoglycin | BC095443 |
| OPTC | opticin | BC074943 |
| ORM1 | orosomucoid 1 | BC143314 |
| ORM2 | orosomucoid 2 | BC056239 |
| OSGIN1 | oxidative stress induced growth inhibitor 1 | BC113417 |
| OSM | oncostatin M | BC011589 |
| OTOR | otoraplin | BC101688 |
| OXT | oxytocin | BC101843 |
| P4HB | prolyl 4-hydroxylase, beta polypeptide | BC071892 |
| PAP21 | chromosome 2 open reading frame 7 | BC005069 |
| PC5 | proprotein convertase subtilisin/kexin type 5 | BC012064 |
| PCSK1 | proprotein convertase subtilisin/kexin type 1 | BC136486 |
| PCSK1N | proprotein convertase subtilisin/kexin type 1 inhibitor | BC002851 |
| PCSK2 | proprotein convertase subtilisin/kexin type 2 | BC005815 |
| PCSK6 | proprotein convertase subtilisin/kexin type 6 | NM_138322 |
| PCSK9 | proprotein convertase subtilisin/kexin type 9 | BC166619 |
| PDCD1L1 | CD274 molecule | BC074984 |
| PDGF2 | platelet-derived growth factor beta polypeptide(simian sarcoma | BC077725 |
| | viral (v-sis) oncogene homolog) | |
| PDGFA | PDGFA associated protein 1 | BC007873 |
| PDGFB | platelet-derived growth factor beta polypeptide(simian sarcoma | BC077725 |
| | viral (v-sis) oncogene homolog) | |
| PDGFC | platelet derived growth factor C | BC136662 |
| PDYN | prodynorphin | BC026334 |
| PECAM1 | platelet/endothelial cell adhesion molecule | BC051822 |
| PENK | proenkephalin | BC032505 |
| PF4 | platelet factor 4 | BC112093 |
| PF4V1 | platelet factor 4 variant 1 | BC130657 |
| PGC | progastricsin (pepsinogen C) | BC073740 |
| PGCP | plasma glutamate carboxypeptidase | BC020689 |
| PGF | placental growth factor | BC007789 |
| PGLYRP1 | peptidoglycan recognition protein 1 | BC096155 |
| PI3 | peptidase inhibitor 3 | BC010952 |
| PIP | prolactin-induced protein | BC010951 |
| PLA2G10 | phospholipase A2, group X | BC106732 |
| PLA2G12 | phospholipase A2, group XIIB | BC143532 |
| PLA2G1B | phospholipase A2, group IB | BC106726 |
| PLA2G2A | phospholipase A2, group IIA(platelets, synovial fluid) | BC005919 |
| PLA2G2D | phospholipase A2, group IID | BC025706 |
| PLA2G2E | phospholipase A2, group IIE | BC140240 |
| PLA2G2F | phospholipase A2, group IIF | BC156847 |
| PLA2G3 | phospholipase A2, group III | BC025316 |
| PLA2G4B | JMJD7-PLA2G4B | BC172355 |
| PLA2G5 | phospholipase A2, group V | BC036792 |
| PLA2G7 | phospholipase A2, group VII | BC038452 |
| PLAT | plasminogen activator, tissue | BC002795 |
| PLG | plasminogen | BC060513 |
| PLGL | plasminogen-like protein | HUMPLGL |
| PLTP | phospholipid transfer protein | BC019898 |
| PLUNC | palate, lung and nasal epithelium associated | BC012549 |
| PMCH | pro-melanin-concentrating hormone | BC018048 |
| PNLIPRP | | |
| PNOC | prepronociceptin | BC034758 |
| PON1 | paraoxonase 1 | BC074719 |
| PON3 | paraoxonase 3 | BC070374 |
| POSTN | periostin, osteoblast specific factor | BC106709 |
| PPBP | pro-platelet basic protein | BC028217 |
| PPIA | peptidylprolyl isomerase A (cyclophilin A) | BC137058 |
| PPT1 | palmitoyl-protein thioesterase 1 | BC008426 |
| PPY | pancreatic polypeptide | BC040033 |
| PRB1 | proline-rich protein BstNI subfamily 1 | BC141917 |
| PRB4 | proline-rich protein BstNI subfamily 4 | BC130386 |
| PRELP | proline/arginine-rich end leucine-rich repeat protein | BC032498 |
| PRG2 | proteoglycan 2, bone marrow (natural killer cell activator, | BC005929 |
| | eosinophil granule major basic protein) | |
| PRH | | |
| PRH1 | proline-rich protein HaeIII subfamily 1 | BC133676 |
| PRL | prolactin | BC088370 |
| PROC | protein C (inactivator of coagulation factors Va and VIIIa) | BC034377 |
| PROK1 | prokineticin 1 | BC025399 |
| PROK2 | prokineticin 2 | BC098110 |
| PROS1 | protein S (alpha) | BC015801 |
| PRR4 | proline rich 4 (lacrimal) | BC058035 |
| PRSS1 | protease, serine, 1 (trypsin 1) | BC128226 |
| PRSS2 | protease, serine, 2 (trypsin 2) | BC103997 |
| PRSS3 | protease, serine, 3 | BC069476 |
| PRSS8 | protease, serine, 8 | BC001462 |
| PSAP | prosaposin | BC001503 |
| PSG11 | pregnancy specific beta-1-glycoprotein 11 | BC020711 |
| PSG3 | pregnancy specific beta-1-glycoprotein 3 | BC005924 |
| PSG4 | pregnancy specific beta-1-glycoprotein 4 | BC063127 |
| PSPN | persephin (PSPN) | BC152717 |
| PTGDS | prostaglandin D2 synthase 21 kDa (brain) | BC005939 |
| PTH | parathyroid hormone | BC096144 |
| PTHLH | parathyroid hormone-like hormone | BC005961 |
| PTN | pleiotrophin | BC005916 |
| PTX3 | pentraxin-related gene, rapidly induced by IL-1 beta | BC039733 |
| PVR | poliovirus receptor | BC015542 |
| PYY | peptide YY | BC041057 |
| QSOX1 | quiescin Q6 sulfhydryl oxidase 1 | BC017692 |
| RAB35 | RAB35, member RAS oncogene family | BC015931 |
| RBP4 | retinol binding protein 4, plasma | BC020633 |
| REG1A | regenerating islet-derived 1 alpha | BC005350 |
| REG1B | regenerating islet-derived 1 beta | BC027895 |
| REG3A | regenerating islet-derived 3 alpha | BC036776 |
| REN | renin | BC047752 |
| RETN | resistin | BC101560 |
| RETNLB | resistin like beta | BC069318 |
| RFNG | RFNG O-fucosylpeptide 3-beta-N-acetylglucosaminyltransferase | BC146805 |
| RFRP | neuropeptide VF precursor (NPVF) | BC160068 |
| RHCE | Rh blood group, CcEe antigens | BC139905 |
| RHD | Rh blood group, D antigen | BC139922 |
| RLN1 | relaxin 1 | BC005956 |
| RLN2 | relaxin 2 | BC126415 |
| RLN3 | relaxin 3 | BC140935 |
| RNASE2 | ribonuclease, RNase A family, 2 (liver, eosinophil-derived | BC096059 |
| | neurotoxin) | |
| RNASE3 | ribonuclease, RNase A family, 3 (eosinophil cationic protein) | BC096061 |
| RNASE6 | ribonuclease, RNase A family, k6 | BC020848 |
| RNASE7 | ribonuclease, RNase A family, 7 | BC074960 |
| RNASET2 | ribonuclease T2 | BC001819 |
| RNH1 | ribonuclease/angiogenin inhibitor 1 | BC014629 |
| RNPEP | arginyl aminopeptidase (aminopeptidase B) | BC001064 |
| RS1 | retinoschisin 1 (RS1) | BC140343 |
| RTN3 | reticulon 3 (RTN3) | BC148632 |
| S100A13 | S100 calcium binding protein A13 | BC070291 |
| S100A14 | S100 calcium binding protein A14 | BC005019 |
| S100A3 | S100 calcium binding protein A3 | BC012893 |
| S100A7 | S100 calcium binding protein A7 | BC034687 |
| SAA1 | serum amyloid A1 | BC007022 |
| SAA4 | serum amyloid A4, constitutive | BC007026 |
| SCDGF-B | platelet derived growth factor D | BC030645 |
| SCG2 | secretogranin II (chromogranin C) | BC022509 |
| SCG3 | secretogranin III | BC014539 |
| SCGB1A1 | secretoglobin, family 1A, member 1 (uteroglobin) | BC004481 |
| SCGB1D1 | secretoglobin, family 1D, member 1 | BC069289 |
| SCGB1D2 | secretoglobin, family 1D, member 2 | BC104838 |
| SCGB3A1 | secretoglobin, family 3A, member 1 | BC072673 |
| SCRG1 | scrapie responsive protein 1 | BC152791 |
| SCUBE1 | signal peptide, CUB domain, EGF-like 1 | BC156731 |
| SCUBE3 | signal peptide, CUB domain, EGF-like 3 | BC052263 |
| SCYE1 | small inducible cytokine subfamily E, member 1 | BC014051 |
| SDCBP | syndecan binding protein (syntenin) | BC143915 |
| SDF1 | | |
| SDF2 | | |
| SECTM1 | secreted and transmembrane 1 | BC017716 |
| SELE | selectin E | BC142677 |
| SELP | selectin P | BC068533 |
| SELPLG | selectin P ligand | BC029782 |
| SELS | selenoprotein S | BC107774 |
| SEMA3A | sema domain, immunoglobulin domain (Ig), short basic domain, | BC111416 |
| | secreted, (semaphorin) 3A | |
| SEMA3B | sema domain, immunoglobulin domain (Ig), short basic domain, | BC013975 |
| | secreted, (semaphorin) 3B | |
| SEMA3E | sema domain, immunoglobulin domain (Ig), short basic domain, | BC140706 |
| | secreted, (semaphorin) 3E | |
| SEMA3F | sema domain, immunoglobulin domain (Ig), short basic domain, | BC042914 |
| | secreted, (semaphorin) 3F | |
| SEMG1 | semenogelin I | BC055416 |
| SEMG2 | semenogelin II | BC070306 |
| SEPN1 | selenoprotein N, 1 | BC156071 |
| SEPP1 | selenoprotein P, plasma, 1 | BC046152 |
| SERPINA | | |
| SERPINC | | |
| SERPIND | | |
| SERPINE | | |
| SERPING | | |
| SFN | stratifin | BC000329 |
| SFRP1 | secreted frizzled-related protein 1 | BC036503 |
| SFRP4 | secreted frizzled-related protein 4 | BC047684 |
| SFRP5 | secreted frizzled-related protein 5 | BC050435 |
| SFTPD | surfactant protein D | BC022318 |
| SHBG | sex hormone-binding globulin | BC112186 |
| SHH | SHH protein | BC111925 |
| SIVA1 | SIVA1, apoptosis-inducing factor | BC034562 |
| SLURP1 | secreted LY6/PLAUR domain containing 1 | BC105135 |
| SMPDL3A | sphingomyelin phosphodiesterase, acid-like 3A | BC018999 |
| SMR3A | submaxillary gland androgen regulated protein 3A | BC140927 |
| SMR3B | submaxillary gland androgen regulated protein 3B | BC144529 |
| SOCS2 | suppressor of cytokine signaling 2 | BC010399 |
| SOD1 | superoxide dismutase 1 | NM_000454 |
| SPACA1 | sperm acrosome associated 1 | BC029488 |
| SPACA3 | acrosomal vesicle protein 1 | BC014588 |
| SPAG11B | sperm associated antigen 11B | BC160085 |
| SPARC | secreted protein, acidic, cysteine-rich (osteonectin) | BC008011 |
| SPC | | |
| SPINT1 | serine peptidase inhibitor, Kunitz type 1 | BC018702 |
| SPINT2 | serine peptidase inhibitor, Kunitz type 2 | BC007705 |
| SPN | sialophorin | BC012350 |
| SPOCK2 | sparc/osteonectin, cwcv and kazal-like domains proteoglycan | BC023558 |
| | (testican) 2 | |
| SPP1 | secreted phosphoprotein 1 | BC093033 |
| SPP2 | secreted phosphoprotein 2 | BC069401 |
| SPRED1 | sprouty-related, EVH1 domain containing 1 | BC137481 |
| SPRED2 | sprouty-related, EVH1 domain containing 2 | BC136334 |
| SRGN | serglycin | BC015516 |
| SST | somatostatin | BC032625 |
| STATH | statherin | BC067219 |
| STC1 | stanniocalcin 1 | BC029044 |
| STC2 | stanniocalcin 2 | BC006352 |
| SULF1 | sulfatase 1 | BC068565 |
| SULF2 | sulfatase 2 | BC110539 |
| TAC1 | tachykinin, precursor 1 | BC018047 |
| TAC3 | tachykinin 3 | BC032145 |
| TCN2 | transcobalamin II; macrocytic anemia | BC001176 |
| TDGF1 | teratocarcinoma-derived growth factor 1 | BC067844 |
| TF | transferrin | BC059367 |
| TFF1 | trefoil factor 1 | BC032811 |
| TFF2 | trefoil factor 2 | BC032820 |
| TFF3 | trefoil factor 3 (intestinal) | BC017859 |
| TFPI | tissue factor pathway inhibitor (lipoprotein-associated coagulation | BC015514 |
| | inhibitor) | |
| TFPI2 | tissue factor pathway inhibitor 2 | BC005330 |
| TFRC | transferrin receptor (p90, CD71) | BC001188 |
| TGFA | transforming growth factor, alpha | BC005308 |
| TGFB1 | transforming growth factor, beta 1 | BC022242 |
| TGFB2 | transforming growth factor, beta 2 | BC096235 |
| TGFB3 | transforming growth factor, beta 3 | BC018503 |
| TGFBI | transforming growth factor, beta-induced, 68 kDa | BC000097 |
| THBS3 | thrombospondin 3 | BC113847 |
| THBS4 | thrombospondin 4 | BC050456 |
| TIMP1 | TIMP metallopeptidase inhibitor 1 | BC000866 |
| TIMP4 | TIMP metallopeptidase inhibitor 4 | BC010553 |
| TINAG | tubulointerstitial nephritis antigen | BC070278 |
| TINAGL1 | tubulointerstitial nephritis antigen-like 1 | BC064633 |
| TLL1 | tolloid-like 1 | BC136429 |
| TLL2 | tolloid-like 2 | BC112341 |
| TMPO | thymopoietin | BC053675 |
| TMPRSS1 | hepsin | BC025716 |
| TNF | tumor necrosis factor (TNF superfamily, member 2) | BC028148 |
| TNFAIP2 | tumor necrosis factor, alpha-induced protein 2 | BC128449 |
| TNFSF1 | lymphotoxin alpha (TNF superfamily, member 1) | BC034729 |
| TNFSF4 | tumor necrosis factor (ligand) superfamily, member 4 | BC041663 |
| TNFSF7 | tumor necrosis factor (ligand) superfamily, member 7 | EF064709 |
| TNFSF8 | tumor necrosis factor (ligand) superfamily, member 8 | BC111939 |
| TNFSF9 | tumor necrosis factor (ligand) superfamily, member 9 | BC104805 |
| TNFSF10 | tumor necrosis factor (ligand) superfamily, member 10 | BC032722 |
| TNFSF11 | tumor necrosis factor (ligand) superfamily, member 11 | BC074823 |
| TNFSF12 | tumor necrosis factor (ligand) superfamily, member 12 | BC071837 |
| TNFSF13 | tumor necrosis factor (ligand) superfamily, member 13 | BC008042 |
| TNFSF14 | tumor necrosis factor (ligand) superfamily, member 14 | NM_003807 |
| TNFSF15 | tumor necrosis factor (ligand) superfamily, member 15 | BC104463 |
| TNFSF18 | tumor necrosis factor (ligand) superfamily, member 18 | BC112032 |
| TNXB | tenascin XB | BC125114 |
| TPSB2 | tryptase beta 2 | BC074974 |
| TPT1 | tumor protein, translationally-controlled 1 | BC003352 |
| TRAP1 | TNF receptor-associated protein 1 | BC023585 |
| TRH | thyrotropin-releasing hormone | BC110515 |
| TRIP6 | thyroid hormone receptor interactor 6 | BC002680 |
| TSHB | thyroid stimulating hormone, beta | BC069298 |
| TSLP | thymic stromal lymphopoietin | BC040592 |
| TTR | transthyretin | BC020791 |
| TUFT1 | tuftelin 1 | BC008301 |
| TWSG1 | twisted gastrulation homolog 1 (Drosophila) | BC020490 |
| TXLNA | taxilin alpha | BC103824 |
| TYMP | thymidine phosphorylase | BC052211 |
| UCN | urocortin | BC104471 |
| UCN2 | urocortin 2 | BC002647 |
| UTP11L | UTP11-like, U3 small nucleolar ribonucleoprotein, (yeast) | BC005182 |
| UTS2 | urotensin 2 | BC126443 |
| VCAM1 | vascular cell adhesion molecule 1 | BC068490 |
| VEGF | | |
| VEGFA | vascular endothelial growth factor A | BC172307 |
| VEGFB | vascular endothelial growth factor B | BC008818 |
| VEGFC | vascular endothelial growth factor C | BC063685 |
| VGF | VGF nerve growth factor inducible | BC044212 |
| VPREB1 | pre-B lymphocyte 1 | BC152786 |
| VTN | vitronectin | BC005046 |
| VWC2 | von Willebrand factor C domain containing 2 | BC110857 |
| WFDC1 | WAP four-disulfide core domain 1 | BC029159 |
| WFDC12 | WAP four-disulfide core domain 12 | BC140217 |
| WFDC2 | WAP four-disulfide core domain 2 | BC046106 |
| WISP1 | WNT1 inducible signaling pathway protein 1 | BC074841 |
| WISP3 | WNT1 inducible signaling pathway protein 3 | BC105940 |
| WNT1 | wingless-type MMTV integration site family, member 1 | BC074799 |
| WNT2 | wingless-type MMTV integration site family member 2 | BC078170 |
| WNT2B | wingless-type MMTV integration site family, member 2B | BC141825 |
| WNT3 | WNT3 protein (WNT3) mRNA | BC111600 |
| WNT3A | wingless-type MMTV integration site family, member 3A | BC103922 |
| WNT4 | wingless-type MMTV integration site family, member 4 | BC057781 |
| WNT5A | wingless-type MMTV integration site family, member 5A | BC064694 |
| WNT5B | wingless-type MMTV integration site family, member 5B | BC001749 |
| WNT6 | wingless-type MMTV integration site family, member 6 | BC004329 |
| WNT7A | wingless-type MMTV integration site family, member 7A | BC008811 |
| WNT7B | wingless-type MMTV integration site family, member 7B | BC034923 |
| WNT8A | wingless-type MMTV integration site family, member 8A | BC156844 |
| WNT8B | wingless-type MMTV integration site family, member 8B | BC156632 |
| WNT9A | wingless-type MMTV integration site family, member 9A | BC113431 |
| WNT9B | wingless-type MMTV integration site family, member 9B | BC064534 |
| WNT10A | wingless-type MMTV integration site family, member 10A | BC052234 |
| WNT10B | wingless-type MMTV integration site family, member 10B | BC096353 |
| WNT11 | wingless-type MMTV integration site family, member 11 | BC074790 |
| WNT16 | wingless-type MMTV integration site family, member 16 | BC104945 |
| XCL1 | chemokine (C motif) ligand 1 | BC069817 |
| XCL2 | chemokine (C motif) ligand 2 | BC070308 |
| YARS | tyrosyl-tRNA synthetase | BC004151 |

In certain embodiments of the invention, and to illustrate the practice of the method of the invention with a plurality of peptide-encoded nucleic acids at a lower complexity than is supported by the robustness of the reagents and methods of the invention, libraries comprising about 50,000 peptide-encoded sequences are provided in each of the five lentiviral vector constructs set forth herein. These libraries are prepared by designing about 50,000 peptide template oligonucleotides targeting approximately 2,000 predicted and known extracellular and membrane (extracellular domain) proteins, including TNFα, IL-1β, and flagellin, as positive controls. For each target protein, a redundant scanning set of about 25 peptides with lengths of 20aa (epitope-like) and 50aa (subdomain-like) are designed. For the 50aa peptides, their length is sufficient to match structures of known protein domains and subdomains with stable folds selected from the NCBI Conserved Domain Database. In making a set of such 50K cytokine lentiviral peptide libraries, two pools of 50,000 oligonucleotides are synthesized for the 20aa and 50aa peptide libraries on the surface of glass slides (two custom 55K Agilent custom microarrays with a size of about 100 and 200 nucleotides). An example of the design of oligonucleotides encoding a particular exemplary peptide is shown below.

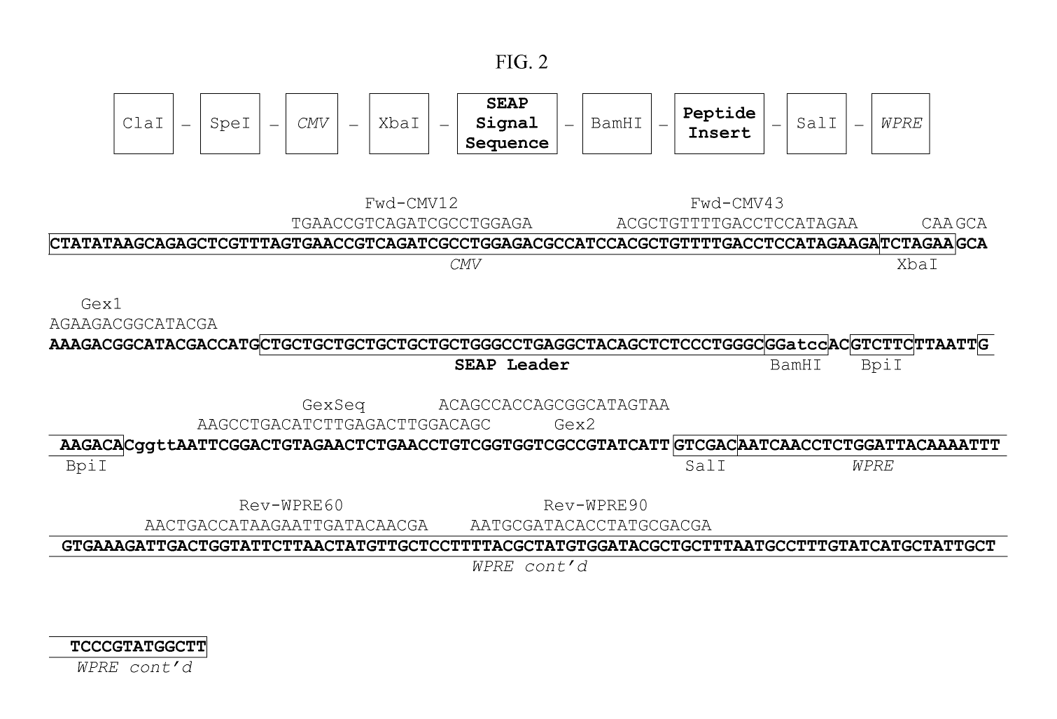

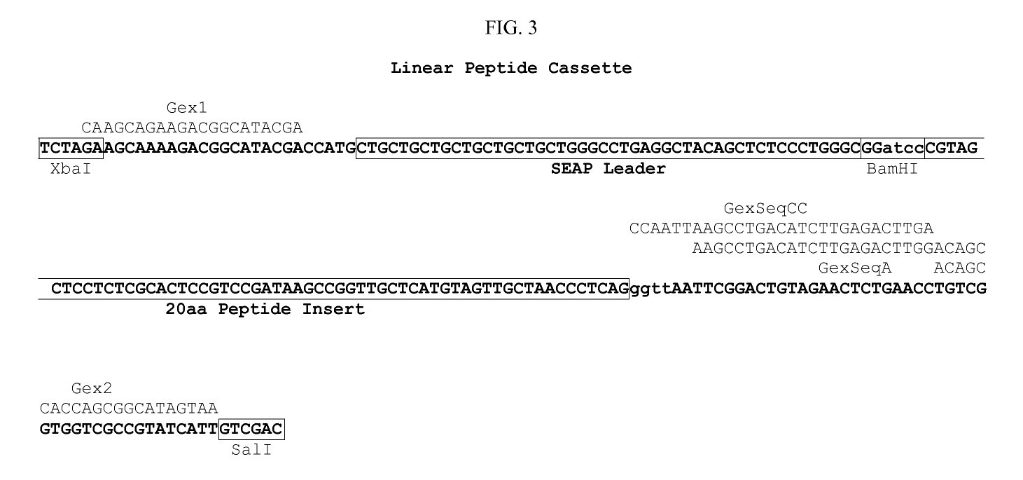

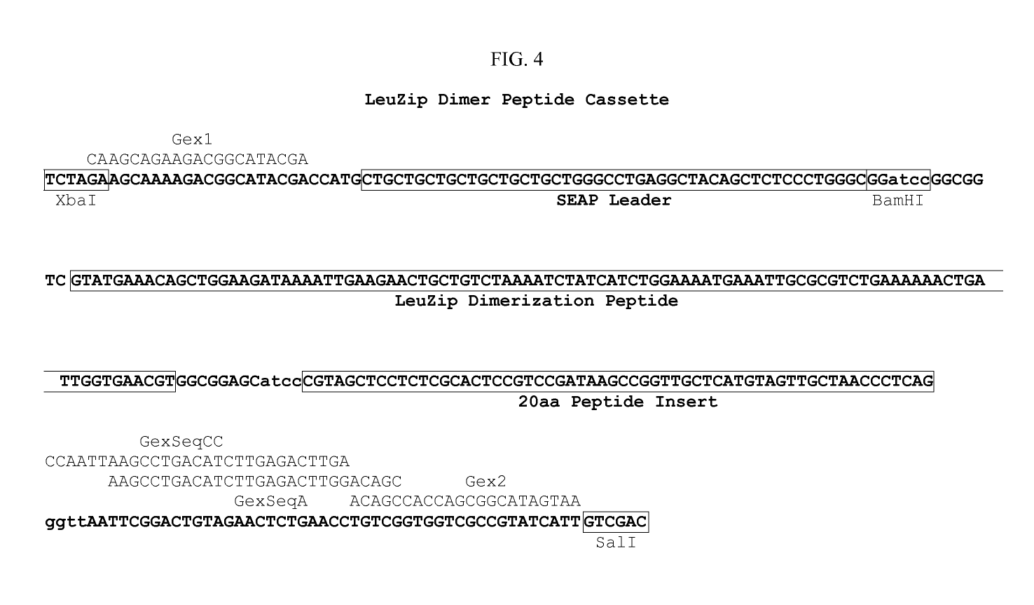

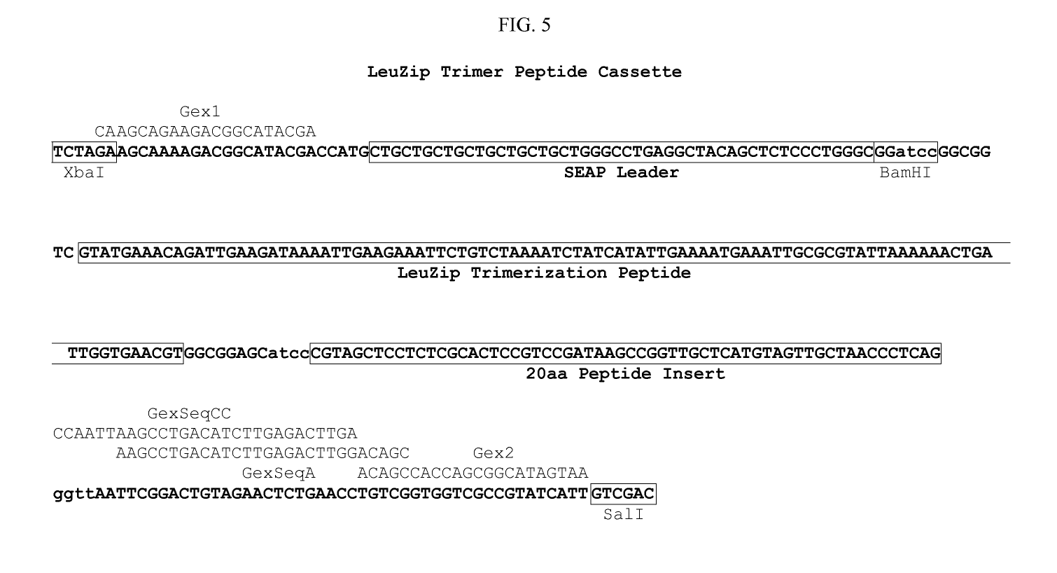

These pools of oligonucleotides are then amplified by PCR (12 cycles) using primers complementary to the common flanking sequences engineered into each oligonucleotide. Amplified peptide cassettes are digested at Bbs I sites engineered into the oligonucleotides and contained in each amplified, peptide-encoding PCR fragment, and each set of fragments amplified from each oligonucleotide pool is cloned into the set of five lentiviral extracellular peptide expression vectors constructed as described herein. As a result of these experiments, five “epitope-like” (20aa) and five “subdomain-like” (50aa) 50K cytokine peptide libraries are provided that express and secrete peptides as monomer, dimer, trimer, cyclic peptide, or membrane-bound on mammalian cell surfaces through the PDGF transmembrane domain. Representation of peptide cassettes in the lentiviral libraries can be ascertained by HT sequencing using, for example, the Solexa (Illumina, San Diego, Calif.) platform (approximately 5×10⁶ reads per sample). Peptide cassettes are amplified using Gex1 and Gex2 flanking vector primers (see, e.g., FIGS. 1-7). The 50K peptide libraries provided as set forth herein can be expected to achieve a representation of at least 95% of the peptides (with less than a 10-fold difference compared to the average abundance level) in the final library. In addition, in each lentiviral peptide library, sequence analysis of 20 randomly selected clones is performed as a quality control check. The libraries are expected to have about a 95% insert rate and less than a 0.2% mutation rate (one mutation in 300 nucleotides) of the peptide inserts.

The construction of 50K receptor peptide ligand libraries representing over 300 well-characterized cytokines, growth factors, chemokines, and hormones is based on recent innovations in HT chip-based oligonucleotide synthesis (200n length) and cloning of peptide cassettes in phage display or viral expression vectors

The invention also provides a set of genome-wide secreted peptide lentiviral libraries that express hundreds of thousands of potentially biologically active receptor peptide ligands rationally designed from all known extracellular and cell-surface proteins of eukaryotic, prokaryotic, and viral genomes. These complex lentiviral secreted peptide libraries, which are highly enriched with functional peptide motifs and subdomain folds that are evolutionarily selected, can be advantageously developed in pooled formats that are compatible with in vitro cell-based functional selection assays. The peptide effectors modulating receptor-mediated cell signaling pathways in functional screens are then identified by HT sequencing.

The peptides identified using the reagents and methods of the invention as set forth herein also provide the basis for peptide-based drugs. New technologies improve the stability, longevity, and targeting of peptides in the body via their modification with various soluble polymers (e.g., polyethylene glycol), the addition of a group that adheres to serum albumin or other serum proteins, their incorporation into protein scaffold microparticular drug carriers, and the use of targeting moieties, transduction peptides, and proteins (see, e.g., Lorens et al., 2000; Torchilin and Lukyanov, 2003, Drug Discov. Today 8: 259-65; Sato et al., 2006, Curr. Opin. Biotechnol. 17: 638-42; Duncan and McGregor, 2008, Curr. Opin. Pharmacol. 8: 616-19). For example, the PEGylated peptide erythropoietin agonist Hematide developed by Affymax has completed Phase II clinical trials (Stead et al., 2006, Blood 108: 1830-34). Significant extension of the serum half-life was achieved by fusion of the AMG 531 (Vaccaro et al., 2005, Nat. Biotechnol. 23: 1283-88), Enbrel (Bitonti and Dumont, 2006, Adv. Drug Deliv. Rev. 58: 1106-18) and CovX peptides (Abraham et al., 2007, Proc. Natl. Acad. Sci. U.S.A. 104: 5584-89) to the antibody Fc domain or to albumin (albumin-interferon a fusion; Subramanian et al., 2007, Nat. Biotchnol. 25: 1411-19).

It is often advantageous to express peptides (peptide aptamers) in the context of a protein scaffold to increase their half-life, limit the number of possible configurations and, in most cases, also improve their binding affinity (Binz et al., 2005; Hosse et al., 2006; Skerra, 2007). A good scaffold should be nontoxic, inert, and soluble, be expressed in a variety of cells, and retain its conformation after insertion of the fused peptide. The first protein scaffold based on the active site loop of E. coli thioredoxin was used to express a combinatorial library of constrained peptides, with the subsequent use of two hybrid systems to select peptides bound to human cdk2 (Colas et al., 1996, Nature 380: 548-50). The GFP, Staphylococcal nuclease, and immunoglobulin chains have been extensively used to express constrained short peptides (Binz et al., 2005; Hosse et al., 2006; Skerra, 2007). Several naturally occurring scaffolds such as leucine zipper and Ig-like domains have also been employed for expression of peptide mimetics of large proteins (Binz et al., 2005; Hosse et al., 2006; Li et al., 2006; Skerra, 2007). Considerable commercial interest is now focused on the use of small scaffolds such as affibodies (Affibody), affilins (Sci1 Proteins), avidins (Avida), anticalins (Pieris), adNectins (Compound Therapeutics), and Kunitz domains (Dyax) (Binz et al., 2005; Lader and Ley, 2001). Additional embodiments of peptide-based drugs that overcome the limitations of stability and delivery are peptidomimetics and non-peptide therapeutics. Peptidomimetics, the process of replacing genetically encoded amino acids with other non-natural molecular residues, is often capable of increasing the plasma stability of peptides by preventing their cleavage by proteases (Ladner et al., 2004). For peptidomimetic design, it is also advantageous to have the smallest possible constrained peptide ligand in terms of conformation (Kay et al., 1998). Typically, the binding strength and stability of a peptide sequence to its target is enhanced when the peptides are cyclized by intramolecular disulfide bonds (Uchiyama et al., 2005, J. Biosci Bioeng. 99: 448-56). Such peptides have been developed, for example, as ligands for integrins and the TNF receptor (Kay et al., 1998).

Peptide leads have traditionally been derived from three sources: natural protein/peptides, synthetic peptide libraries, and recombinant libraries. As potential therapeutics, peptides offer several advantages over small molecules (increased specificity and affinity, low toxicity) and antibodies (small size). Germane to the invention, nearly all peptide therapeutics developed thus far have been derived from natural sources. In contrast, peptides derived from random peptide recombinant libraries (phage, ribosome, cell surface display, etc.) have received little commercial interest due to difficulties in developing therapeutics with pharmacological properties comparable to natural peptides (Mersich and Jungbauer, 2008; Duncan and McGregor, 2008; Sato et al., 2006). This is likely due, in part, to the result that screens of randomly-encoded peptide libraries for blockers of protein interactions usually exhibit very low (1/100,000-1/1,000,000) hit rates (Watt, 2006). These low hit rates may reflect the fact that many peptides in randomly encoded libraries may be incapable of adopting a stable conformation unless artificially constrained in a manner that limits its potential for structural diversity. While in principle it should be possible to derive stably folded structures from random libraries of peptide sequences selected through phage or ribosome display screens, in practice this has turned out to be a daunting task. Even the largest libraries ever constructed (with complexities of 10¹²) do not have the complexity to cover even a small fraction of the possible variants of such peptides (12²⁰ or 8×10²⁶ for a 12aa epitope-like peptide pool).

The pharmacological properties of peptide dendrimers (i.e., branched peptides or multiple antigen peptides) provide a unique opportunity to develop novel classes of highly effective drugs. Due to their small size, peptide dendrimers can be effectively delivered to tissues (more efficiently than antibodies), and are less immunogenic than recombinant proteins and antibodies. Moreover, peptide dendrimers are remarkably stable in vivo (up to several days in plasma or serum) due to low renal clearance and high resistance to most proteases and peptidases (Pini et al., 2008, Curr. Protein Peptide Sci. 9: 468-77; Niederhafner et al., 2005, J. Peptide Sci. 11: 757-88; Sadler et al., 2002, J. Biotechnol. 90: 195-229; Boas et al., 2004, Chem. Soc. Rev. 33: 43-63; Dykes et al., 2001, J. Chem. Technol. Biotechnol. 76: 903-18; Yu et al., 2009, Adv. Exp. Med. Biol. 611: 539-40; Tam et al., 2002, Eur. J. Biochem. 269: 923-32; Orzaez et al., 2009, Chem. Med. Chem. 4: 146-60; Falciani et al., 2009, Expert Opin. Biol. Ther. 9: 171-78). Moreover, multimerization of peptide ligands by dendrimeric scaffolds significantly increases their agonistic or antagonistic activity against specific receptors (from the μM to nM range), as demonstrated for DR5 (Li et al., 2006), CD40 (Orzaez et al., 2009), Erb1 (Fatah et al., 2006, Int. J. Cancer 119: 2455-63), ERBB-2 (Houimel et al., 2001, Int. J. Cancer 92: 748-55), and several other TNF death receptors (Wyzgol et al., 2009, J. Immunology 183: 1851-61). HTS with dendrimeric peptides (i.e., trimers and tetramers) can yield approximately 100-fold more hits than screening with monomeric peptides. The outstanding activity of dendrimeric peptides can be explained by an increase in local peptide concentration and enhanced efficacy of the interaction between preassembled multivalent ligands and multimeric receptors (Orzaez et al., 2009; Miller, 2000; Wyzgol et al., 2009).

### Examples

The description set forth above and the Examples set forth below recite exemplary embodiments of the invention. The following Examples are intended to further illustrate certain preferred embodiments of the invention and are not limiting in nature.

### Example 1

### Validation of Pentiviral Peptide Libraries for HTS of Bioactive Peptides

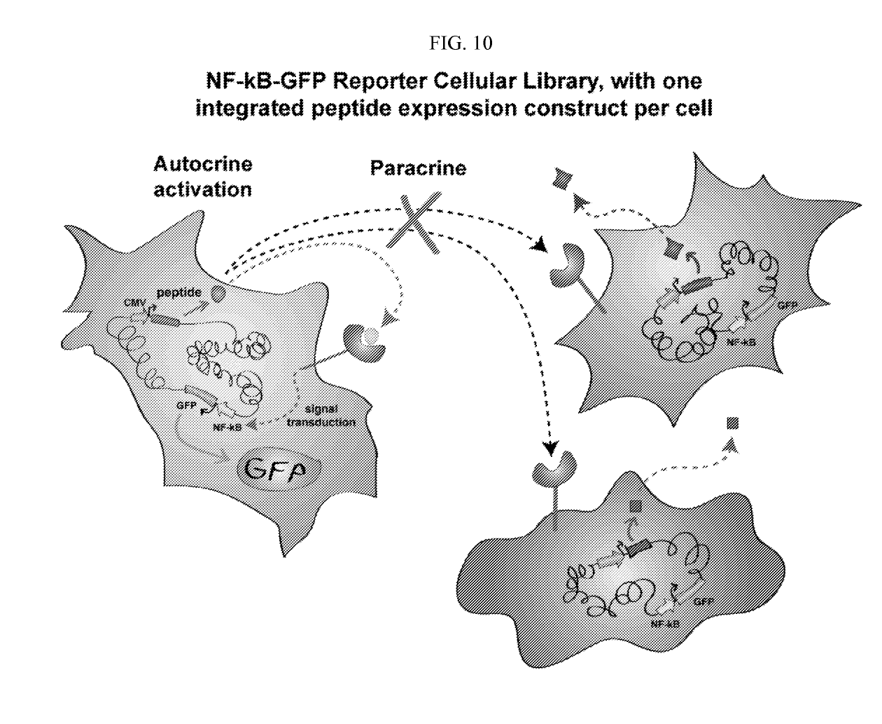

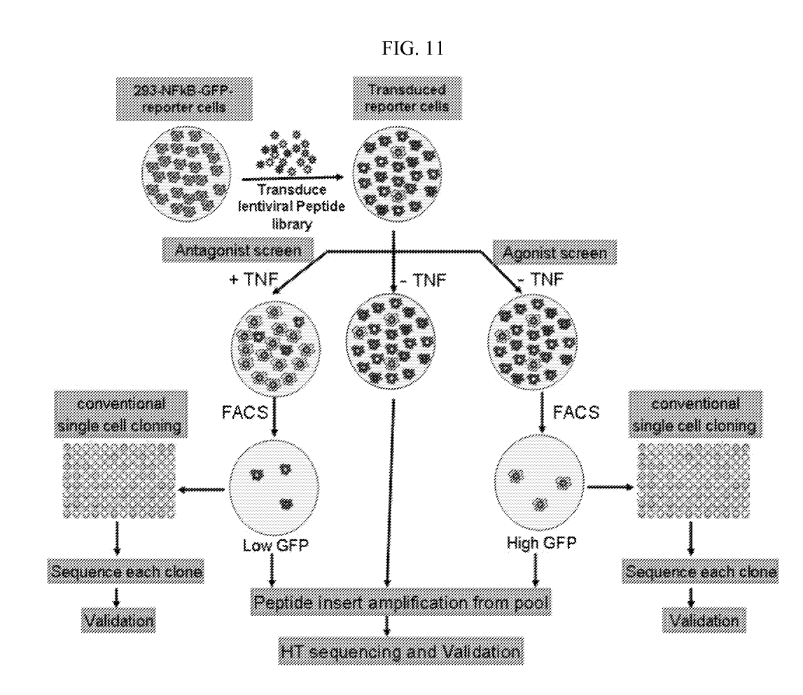

Pooled lentiviral peptide libraries (50K) were validated for the discovery of extracellular peptide effectors of TLR5, TNFα, and IL-1β-receptor mediated NF-κB signaling pathways using a human embryonic kidney cell line (HEK 293) comprising a reporter protein (green fluorescent protein) operatively linked to an NF-κB-responsive promoter as illustrated in FIG. 10. The 293-NFκB reporter cell line was transduced with the peptide libraries. Cell fractions demonstrating a modulation in the GFP reporter expression level, defined as either activation or repression, after induction with natural ligands were isolated by FACS. Bioactive peptides were identified by amplification of peptide cassettes from the genomic DNA of sorted cells, followed by HT Solexa sequencing. This process is depicted schematically in FIG. 11. The peptides identified in the primary screen were then further developed as lentiviral peptide effector constructs and free peptides, and tested for efficacy in modulating NF-κB signaling in vitro and in vivo. In the course of these experiments, the performance of different peptide designs (linear, constrained, monomer, dimer, trimer, scaffold) was compared in functional screens of TLR5, TNFα, and IL-1β receptor ligands. These validation studies were useful for defining optimum performance design (size and scaffold of peptide cassettes) for use in developing a set of commercial 500K secreted peptide libraries.

### Example 2

Development of 500K Secreted Peptide Libraries

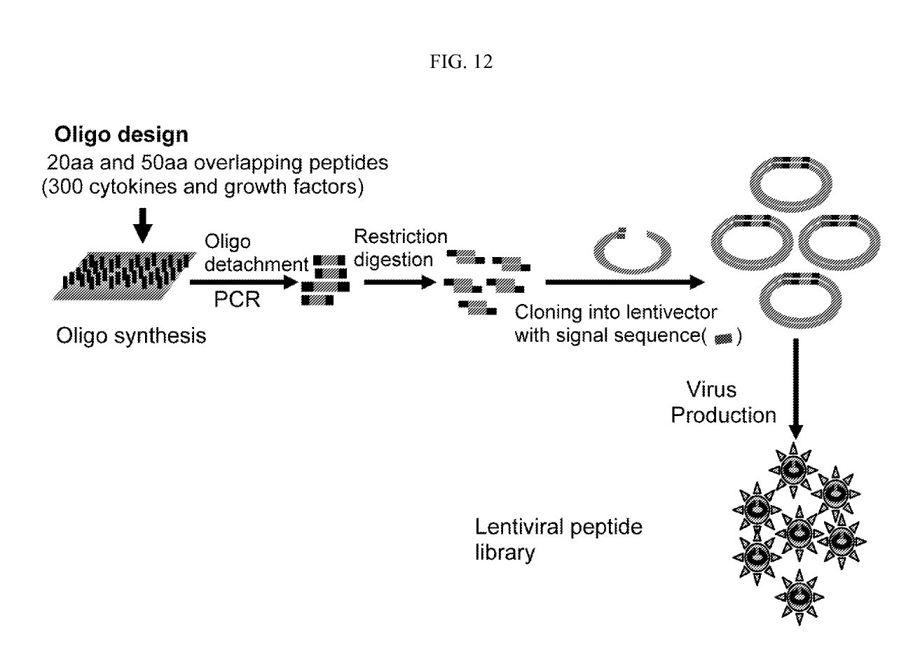

Using computational prediction tools developed as set forth above, a comprehensive set of extracellular proteins of eukaryotic, prokaryotic, and viral origin were selected, including but not limited to cytokines, growth factors, extracellular proteins, matrix proteins, receptors (extracellular domains), membrane-bound proteins, toxins, bioactive proteins/peptides. An exemplary set of such proteins is set forth in Table 1. There are an estimated 25,000 proteins that can act by modulating cellular responses through interactions with cell surface receptors. The selected extracellular protein sequence pool was reduced to a set of protein functional domains that are evolutionarily conserved (an estimated 100,000) using computer-assisted sequence alignment analysis and the NCBI Conservative Domain Database (CDD) as discussed herein. For each selected domain, a redundant set of 2-20 peptides (15aa-60aa in length) was designed to comprise whole small domains or subdomains (for medium-big domains) with stable fold structures. HT oligonucleotide synthesis was used to construct a set of pooled domain/subdomain-like 500K secreted effector lentiviral libraries with constitutive or tet-regulated expression of secreted peptides in the scaffold designs demonstrating the best performance in validation studies as described in Example 1. An example of this experimental design is depicted graphically in FIG. 12. The developed 500K peptide libraries were validated in the functional screen of NF-κB modulators as identified herein.

### Example 3

### Optimization of Functional Screening Strategy Using a Secreted Lentiviral Peptide Library

Some of the limitations of the phage display technology for functional screening can be overcome by directly expressing the peptide library in mammalian cells. Although retroviral expression libraries of cDNA fragments (GSEs) and peptides have been successfully employed in the past to isolate intracellular transdominant negative agents (Roninson et al., 1995; Delaporte et al., 1999; Lorens et al., 2000; Xu et al., 2001), these approaches have in practice been limited to intracellular peptides. Disclosed herein is a secreted peptide library using the lentiviral expression system to enable functional screening of receptor peptide ligands. Such lentiviral secreted peptide libraries, in combination with suitable reporter cells and FACS, can be used to isolate peptide drugs.

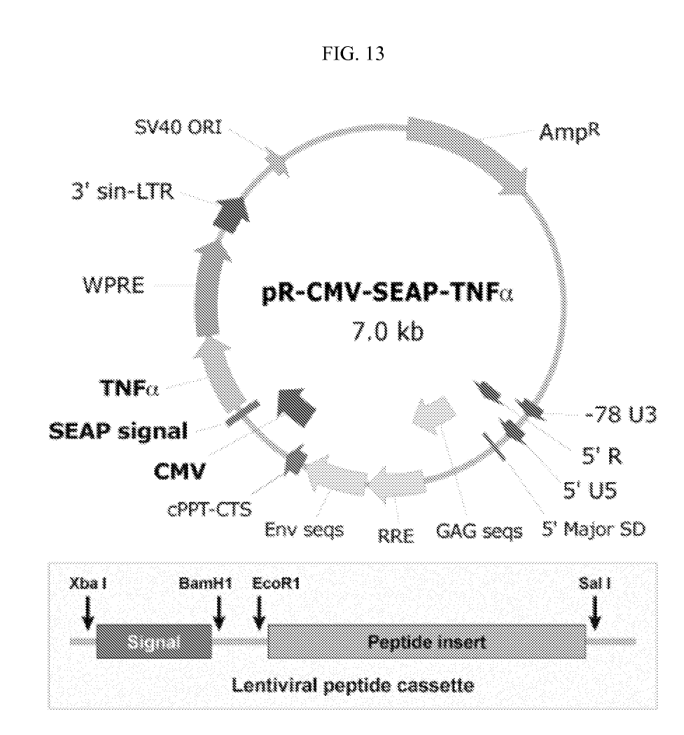

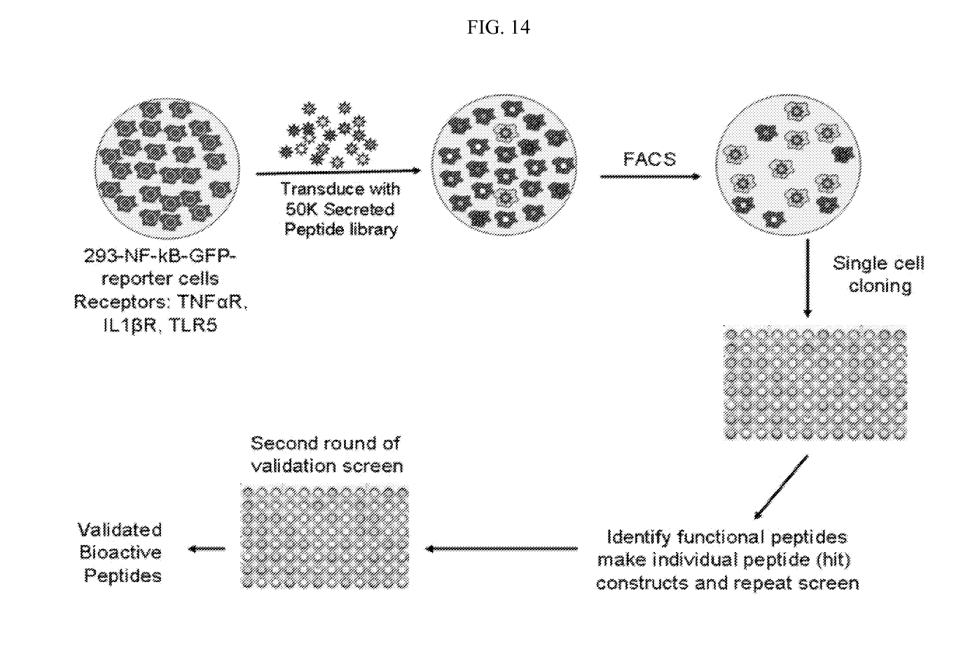

In order to select an optimal signal sequence for peptide secretion, four novel lentiviral secretion vectors were developed containing an IL-1-signal sequence (S1), an improved mutant form of the IL-1-signal sequence (S2), a secreted alkaline phosphatase (S3), and a CD14 signal sequence (S5) in XbaI/BamHI sites of a pR-CMV vector downstream of CMV promoter followed by Kozak sequence and an ATG initiation codon. Full-length cDNAs of TNFα, IL-1β, and flagellin (CBLB502) were then cloned in-frame into EcoRI/SalI sites downstream of each of the four lentiviral secretion vectors, as illustrated in FIG. 13. HEK293 cells were then transduced with all 12 packaged constructs, the media was replaced after 24 hours, and after one passage (to ensure that all residual virus particles were removed), the plates were seeded with 293-NFκB-GFP reporter cells, as shown in FIG. 14. After 24 hours, NF-κB activation in 293-NFκB-GFP by the control proteins (TNF, IL-1, and CBLB502) secreted by HEK293 cells was analyzed by fluorescence microscopy (GFP induction). The pR-CMV-S3 vector with the secreted alkaline phosphatase signal sequence (SEAP) provided the most efficient secretion of all three control proteins, and this vector was selected for development of the peptide libraries.

With secreted peptide libraries, the secreted peptides could affect not only the phenotype of the host cells expressing them (autocrine mechanism), but also the cells in an accessible range of diffusion (paracrine mechanism). Thus, for a successful functional screen using secreted peptide libraries, conditions should be optimized to selectively isolate clones secreting functional receptor ligands from bystander cells that could be modulated by the diffused ligands. To optimize conditions for functional screening of NF-κB agonists, stable clones of the 293-NFκB-GFP reporter cells capable of constitutive TNF secretion were developed. In order to assess the rate of diffusion of the secreted TNF, NF-κB-GFP reporter cells that secrete TNF (therefore GFP-positive) were mixed with an excess (ratio 1:10,000) of reporter cells that do not secrete TNF (GFP-negative). The cells were plated at different densities with and without a 0.6% agarose overlay. GFP-positive clusters were examined by fluorescence microscopy every 24 hours. As expected, at high plating densities (more than 1×10⁴ cells/cm²), distinct clusters of GFP-positive cells were detected only with agar overlay, even after a week, whereas when plating was performed without agar, a large population of cells was GFP-positive due to the diffusion of secreted TNF. Plating cells at low cell densities (2×10³ cells/cm²) without agar resulted in distinct GFP-positive clusters of cells without affecting neighboring cells (shown in

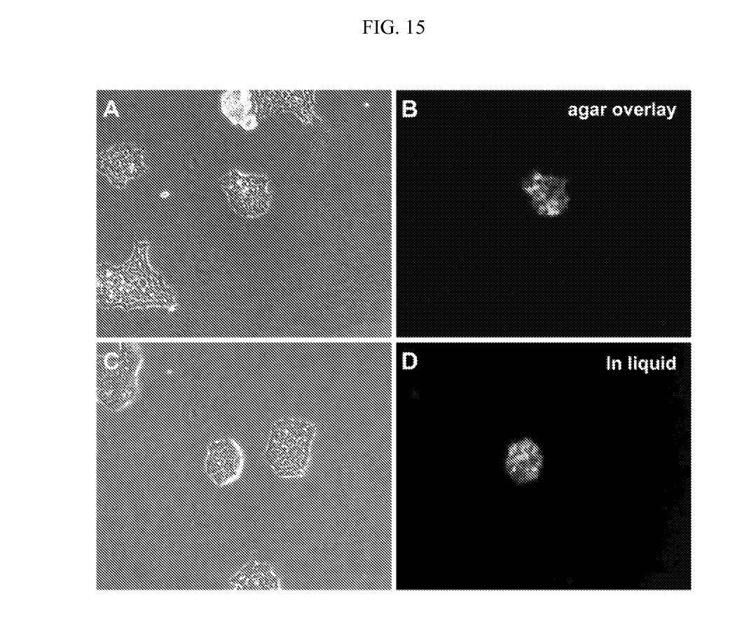

FIG. 15). Cell plating at low densities permitted rapid recovery of the fraction of GFP-positive cells by trypsinization of the entire plate, followed by FACS sorting. In order to demonstrate the feasibility of isolating functional peptides from a pool of bystanders, the TNF-secreting NF-κB-GFP reporter clone was mixed with reporter cells transduced with a control vector at a ratio of 1:10K, and then plated at low density; the resulting GFP-positive cells were sorted. After two rounds of FACS sorting, over 97% of the cells were GFP-positive.

### Example 4

### Secreted Peptide Libraries for Cytokines that Do Not Activate NF-κB

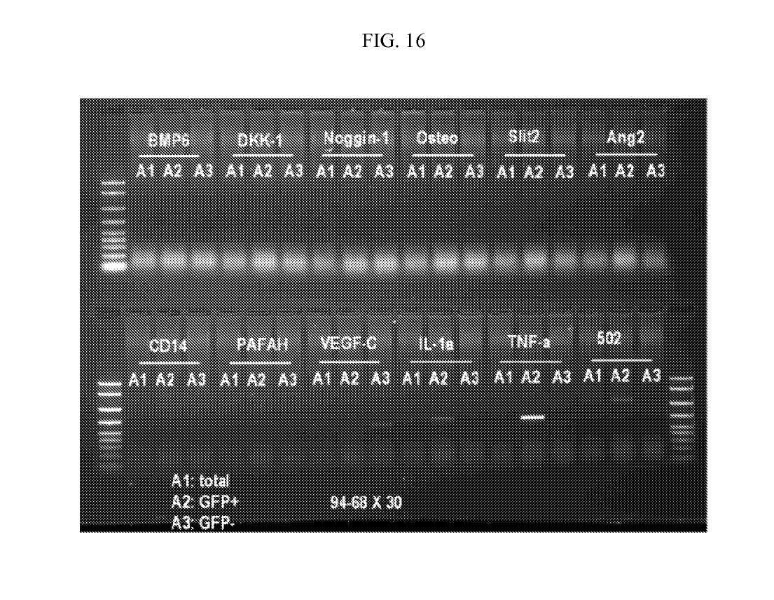

To further demonstrate that functional peptides can be isolated from a complex peptide library, a secreted peptide library was prepared for 10 cytokines that do not activate NF-κB (BMPG, DKK-1, Noggin-1, Osteo, Slit2, Ang2, CD14, PAFAH, and VEGF-C) and three positive control NF-κB agonists (TNF, IL-1, and Flagellin (CBLB502)). These cytokines were mixed with empty vector at a ratio of 1:10K, transduced into NF-κB-GFP reporter cells, and seeded at low density. GFP-positive cells were sorted, and genomic DNA was isolated from total GFP+ and GFP− cell fractions, and then tested by PCR for enrichment of each specific cytokine As shown in FIG. 16, only TNF, IL-1, and 502 were enriched in the GFP+ fraction. After three rounds of FACS sorting, over 95% of the population was GFP-positive, and all single clones isolated from the GFP+ fraction corresponded to the positive controls inserts (TNF, IL-1, and CBLB502)

### Example 5

### Development and Validation of the 50K Secreted Ligand Receptor Lentiviral Library

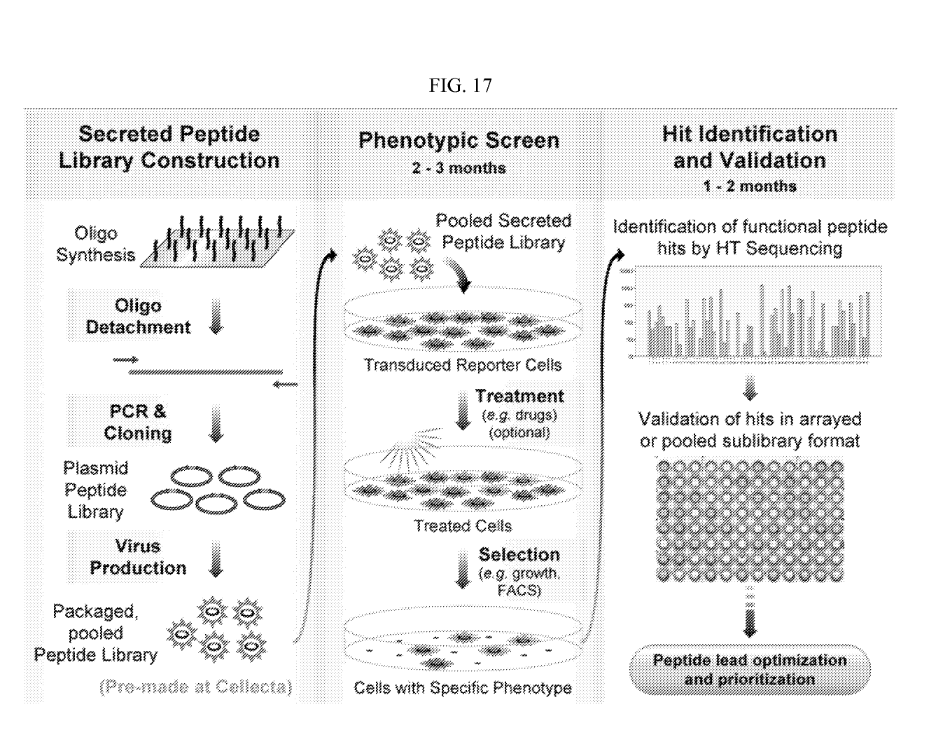

The set of ten 50K cytokine peptide lentiviral libraries prepared as disclosed above were validated and protocols for HTS optimized in cell-based assays. These pooled peptide libraries were screened for the discovery of novel peptide modulators of the NF-κB signaling pathway using the 293-NFκB-GFP transcriptional reporter cell line disclosed herein and as illustrated in FIG. 17. The NF-κB signaling pathway has been shown to play an important role in regulating the immune response, apoptosis, cell-cycle progression, inflammation, development, oncogenesis, viral replication, chemotherapy resistance, tumor invasion, and metastasis (Tergaonkar et al., 2006, Int. J. Biochem. Cell Biol. 38: 1647-53; Graham and Gibson, 2005, Cell Cycle 4: 1342-45; Wu and Kral, 2005, J. Surg. Res. 123: 158-69; Lu and Stark, 2004, Cell Cycle 3: 1114-17). A wide range of modulators, including cytokines (TNFα and IL-1β), mitogens, toxic metals, and viral and bacterial products (e.g., flagellin) activate NF-κB through several families of cell surface receptors (TCRs, IL-1Rs, TNFRs, GF-Rs, TLRs). This extensive knowledge of receptor ligands and intracellular components of the NF-κB signaling pathway increases confidence in predicting the outcomes of test screening assays, and provides a stringent assessment of lentiviral peptide library performance. On the other hand, the different modulators that activate NF-κB signaling are still poorly characterized. Thus, the test screen with the whole set of lentiviral secreted peptide libraries will likely provide insights into unknown receptor activation mechanisms, and may lead to the identification of new pharmacologically promising peptides that modulate the NF-κB signaling pathway. These findings could be used in the development of novel drugs for the treatment of a variety of pathological conditions, including inflammation and cancer.

In order to demonstrate the feasibility of isolating NF-κB modulators from a complex library, a secreted peptide library was prepared using the same pool of oligonucleotides (encoding overlapping scanning sets of 20 aa-long and 50 aa-long peptides for cytokines and extracellular matrix proteins as set forth in Table 1) previously used for construction of the 50K ligand receptor phage display library. These oligonucleotides were cloned in the pR-CMV-SEAP vector downstream of the SEAP signal sequence for linear 50K 20aa and 50aa secreted peptide libraries (FIG. 13). Also constructed were 20aa and 50aa 50K libraries expressing dimeric peptide constructs by cloning leucine zipper dimerization sequence (32aa) (Li et al., 2006) upstream of peptide insert between the EcoRI and BamHI sites (FIG. 13). The basic outline of library construction is depicted in FIG. 12 as discussed herein. Randomly selected clones (40 clones from each library) were chosen and sequenced, revealing that the 20aa peptide libraries contained over 80% correct inserts and the 50aa peptide libraries 40% correct inserts.

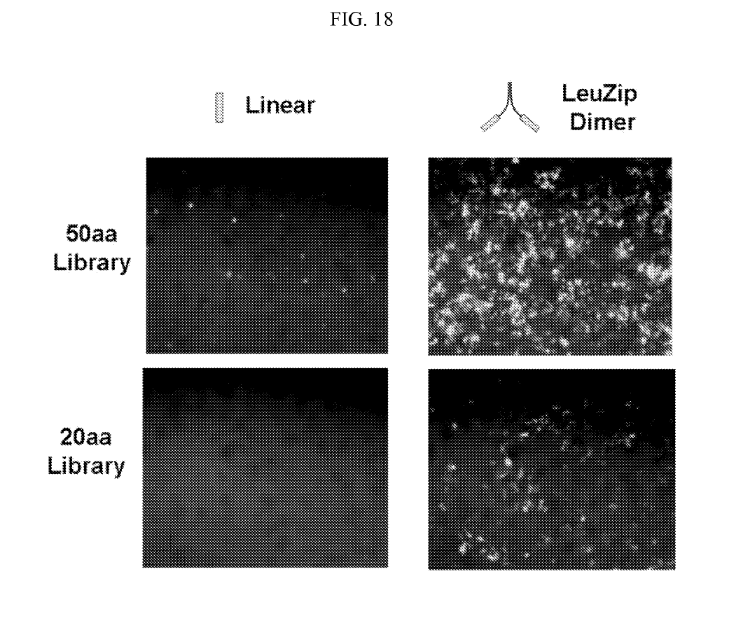

In order to validate the application of the four developed 50K ligand receptor lentiviral peptide libraries (20aa- and 50aa-long) for selection of peptide modulators in functional screens using cell based assays as disclosed above, proof-of-principle screens were performed for agonists of NF-κB signaling using 293-NFκB-GFP reporter cells. Reporter cells (5×10⁶ cells) were transduced with each of the four 50K peptide lentiviral libraries at a multiplicity of infection (MOI) of 0.2, and GFP-positive cells were isolated by FACS after 48 hours. Approximately 0.02% GFP-positive cells (about 2,000 cells) were isolated from the total population (with a background of approximately 0.01-0.02%) in the first round of FACS selection. Sorted GFP-positive cells were plated as single cells in 96-well plates or in bulk in dishes, allowed to grow for an additional two weeks, and analyzed by fluorescent microscopy and FACS. The growth medium was replaced every 24 hours to minimize diffusion of secreted peptides, which could activate bystander cells and lead to false positives. FACS analysis indicated at least a 5-10 fold enrichment (0.1-0.2%) of the clones with activation of NF-κB signaling in the libraries expressing peptide dimers (3-5-fold more GFP-positive clones in the 50aa library as compared with the 20aa library) above the background level of cells transduced with lentiviral vector alone (0.01%). An additional round of FACS sorting clearly demonstrated a significant enrichment of GFP-positive clones (approximately 10%) in the cells expressing dimeric or 50aa linear secreted peptide constructs (FIG. 18).

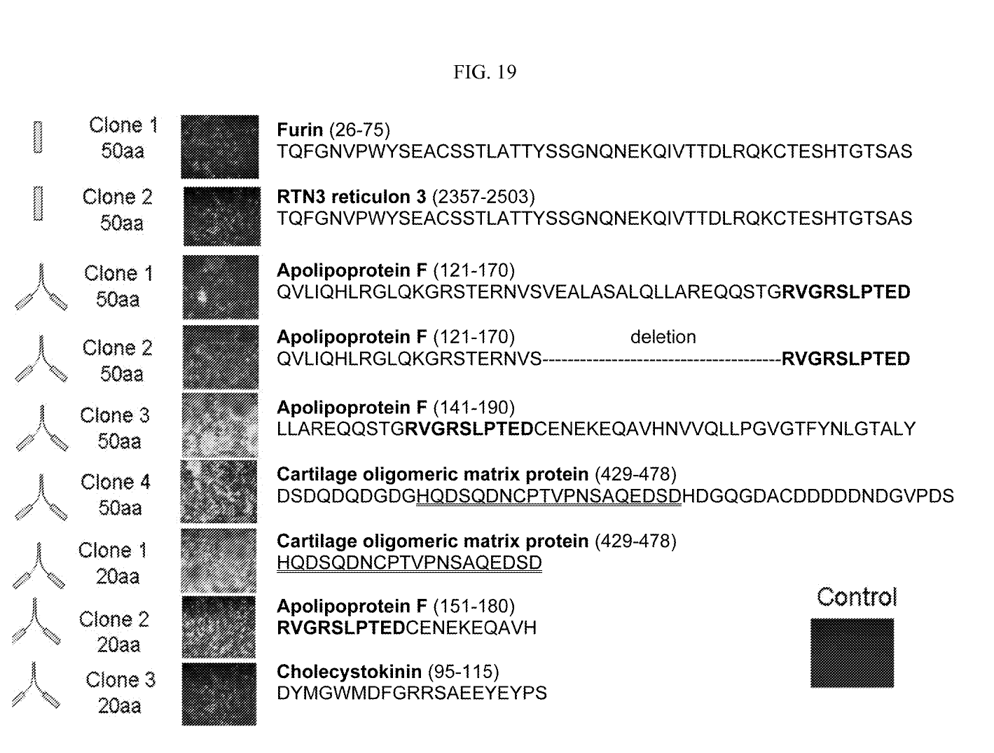

In order to identify specific sequences of peptides that may activate NF-κB signaling, for each library, 20 cell clones were randomly-chosen after one round FACS sorting of the reporter cells transduced with linear and dimeric peptide libraries, the peptide inserts from genomic DNA amplified by two rounds of PCR using flanking vector primers, and functional peptide hits were identified by conventional sequence analysis. FIG. 19 shows the amino acid sequences of the identified novel peptide agonists of NF-κB signaling (two clones from 50aa linear peptide library and seven clones from 20aa and 50aa dimeric peptide libraries).

In order to confirm the peptide hits identified by the first round of screening, nine identified peptide inserts were cloned into the corresponding pR-CMV-SEAP (or pR-CMV-SEAP-LeuZip) lentiviral vector and transduced into 293-NFκB-GFP reporter cells. All nine lentiviral peptide constructs demonstrated clear activation of NF-κB signaling at different levels in the transduced reporter cells (FIG. 19). In additional studies, it was shown that none of the lentiviral peptide constructs identified in the primary screen, but missing the signaling sequence, were able to activate expression of GFP when transduced in NF-κB reporter cells. These confirmation studies ensured that the GFP-positive clones were not false positives due to a bystander effect, and that they do not represent reporter cells that express GFP due to viral integration leading to activation of NF-κB reporter cells.

### Example 6

### Screening for Receptor Agonists and Antagonists of NF-κB Signaling

Several positive control constructs were developed in order to optimize conditions for the functional screening of peptide modulators of NF-κB signaling. Secreted lentiviral constructs expressing full-length TNFα, IL-1β, and flagellin fragment CBLB502 were prepared previously, and the ability of secreted NF-κB agonists to effectively activate NF-κB signaling using 293-NFκB-GFP reporter cells was confirmed. These positive control agonists were then cloned into the set of novel lentiviral vectors developed as set forth herein and used as positive controls in validation studies. In order to optimize conditions for the HTS of NF-κB agonists, plasmid DNA from the positive control and the pooled 50K linear peptide library were mixed at ratio of 1:5,000, packaged, and transduced 10×10⁶ 293-NFκB-GFP reporter cells at an MOI of 0.3-0.5, which yielded about 100 transduced cells for each peptide construct. The transduced reporter cells were then grown for 2 days at low-medium density (5×10³ cells/cm²), sorted for GFP+ cell fractions, grown at low density (2×10³cells/cm²) for an additional 5-7 days, and sorted again for GFP+ cells. Enrichment of the positive control constructs was monitored by RT-PCR using gene-specific primers. In the course of these preliminary HTS screens, transduction (MOI), cell growth conditions (density), the time course of reporter expression, the number of rounds, and FACS sorting gates required to enrich positive controls were optimized. Using these optimized conditions, HTS of novel TLR5, TNFα, and IL-1β receptor ligand peptide agonists were performed with the whole set of ten 50K cytokine peptide libraries developed as described herein. In addition, similar screens were performed for peptide antagonists of the TLR5 receptor by transducing the 50K cytokine libraries into 293-NFκB-GFP reporter cells pre-activated with a suboptimal concentration of flagellin (0.1 pM). In the antagonist screen, two rounds of FACS sorting were performed on GFP-negative cells that had lost GFP reporter activation in response to conditions optimized as described herein. In order to identify novel peptide modulators (agonists or antagonists), genomic DNA from control (transduced cells) and GFP+ or GFP− cells was isolated after the second round of FACS sorting and used for amplification of the peptide cassette with flanking Gex primers, followed by HT Solexa sequencing. Optimized amplification and HT sequencing protocols indicated that at least 5×10⁶ reads from each sample could be expected, averaging about 100 reads for each peptide in the library. If the number of reads was not sufficient to generate statistically significant data (less than 20 reads per peptide), amplified PCR product purification conditions and the concentration of the PCR product at the sequencing stage were optimized or the sequencing scale increased. In order to estimate the reproducibility of these data, each HTS screen with the specific 50K peptide library was repeated three times. Statistical analysis of these data was performed using SPSS v15.0 for Windows and other software to identify a set of peptide modulators (candidates) from the HT sequencing data. These experiments were expected to yield a set of approximately 50-200 peptide agonist and antagonist candidates that were enriched at least three times in at least two duplicate screens in the FACS sorted cell fractions.

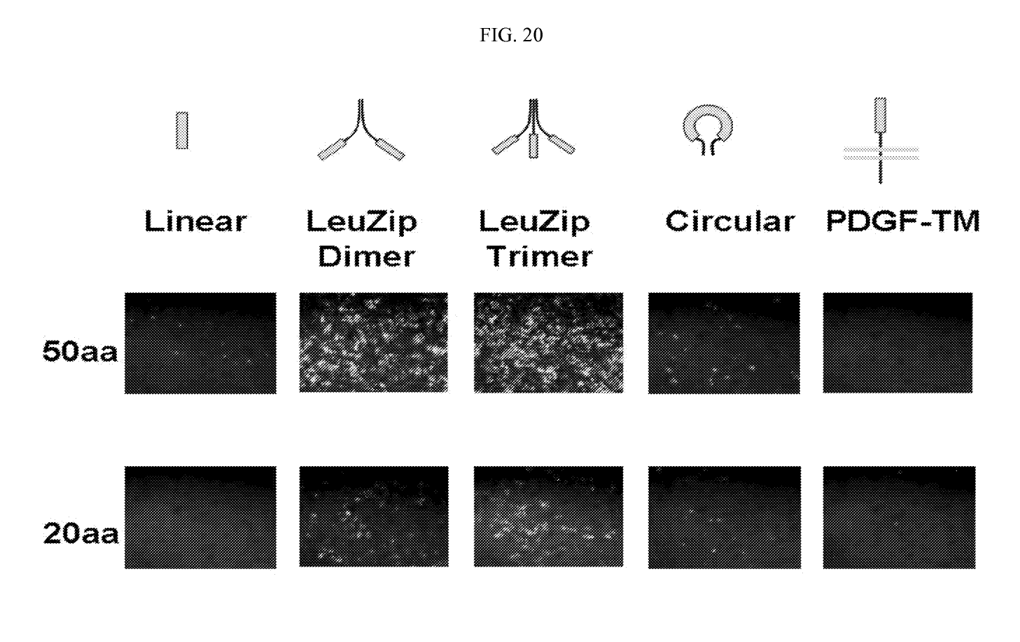

Results of these experiments are shown in FIG. 20, wherein GFP reporter gene activation is seen only using libraries comprising leucine zipper dimer and trimer embodiments, whereas linear, cyclized, and membrane-associated embodiments do not efficiently produce detectable results on the GFP reporter cells.

### Example 7

### Experimental Validation of Functional Peptide Hits Identified in the NF-κB Screens (Second Round of Screening)

In order to validate the results of the HTS screen, the expected set of 50-200 individual lentiviral constructs expressing functional peptide candidates identified in the primary screens described herein was assessed. These peptide constructs were cloned, packaged, and transduced into 293-NFκB-GFP reporter cells in an arrayed format, and then their ability to modulate NF-κB signaling assayed. In additional experiments, the biological activity of the secreted peptides was validated and compared between isolated peptides. To accomplish this goal, validated peptide constructs were cloned into a modified lentiviral vector that allows for expression of the secreted peptides as fusion constructs with well-characterized TEV-Biotin-binding tags (23aa) (Boer et al., 2003, Proc. Natl. Acad. Sci. U.S.A. 100: 7480-85). The peptide constructs were packaged and transduced into HEK293T cells, and the peptide-tags labeled with BirA biotin ligase. The secreted Biotin-Tag-peptides were then purified with streptavidin columns, eluted with TEV protease, and their biological activity measured in a cell-based assay with 293-NFκB-GFP reporter cells. These experiments provide a comparison of the reproducibility, number of true positive hits, and percentage of false positives to facilitate the choice of optimum designs for construction of 500K secreted peptide libraries. In addition, these experiments provide a set of validated, high efficacy peptides (expected to be 10-20 peptides) that effectively modulate NF-κB signaling.

To further understand the mechanism of NF-κB modulation by the discovered novel peptides, digital expression profiling data was performed using HT sequencing in the Solexa platform (Illumina, San Diego, Calif.) for reporter cells treated with natural and validated peptide modulators. The set of differentially expressed genes was first imported for storage and analysis in the Pathway Studio Enterprise software from Ariadne, which combines a collection of greater than 550 Signaling Line pathways, ˜200 canonical pathways, ˜30,000 pathway components, and several thousand Ariadne ontology categories, as well as public gene sets (GO, STKE, KEGG, Broad datasets). These expression data were mapped to known signaling pathways and group natural and novel peptide modulators based on two-dimensional hierarchical clustering using the TMEV software package in several groups based on their mechanism of action. There are expected to be at least three mechanisms of NF-κB modulation induced by natural and novel peptide agonists and antagonists of TLR5, TNFα, and IL-1β receptors resulting from these experiments. In order to confirm the mechanism of action, certain of these regulators (hubs), including TLR5, TNFα, and IL-1β receptors, were used to develop a set of small hairpin RNA (shRNA) constructs against them in a lentiviral vector expressing the puromycin resistance gene. These shRNA constructs were then packaged into lentiviral particles, transduced into 293-NFκB-GFP cells, and selected for three days in puromycin. This cell panel with specific knockdown of cell surface and intracellular NF-κB signaling pathway regulators was then treated with natural and validated peptides and examined for the ability to block activation of the GFP reporter. These data provide validation of upstream (receptor) and downstream key regulators of the NF-κB pathway, serving as a key confirmation of the success of the pooled secreted peptide screens. This identified subset of unique peptides with high TLR5R agonist and antagonist activity were used to initiate a drug development pipeline.

Results from screening assays as set forth herein are shown in Tables 2A and 2B, wherein Table 2A demonstrates that multimerization of peptides significantly increases the percentage of true positive hits obtained for particular peptide constructs (wherein “+” indicates that there was at least a 10-fold of the peptide construct above basal level after two rounds of selection for GFP-positive cells in HEK293-NFκB-GFP transcriptional reporter cells transduced with lentiviral peptide library and “−” indicates that there was no enrichment of the peptide construct) and Table 2B shows the nucleotide and amino acid sequences of the peptide identified in the screen.

TABLE 2A

| | Trimer | Dimer | Linear | Fusion | Cyclic |
|---|---|---|---|---|---|
| Gene Name | 50aa | 50aa | 50aa | 50aa | 50aa |
| PF4V1 | + | − | | | |
| CCK | + | + | | | |
| NPPA | + | − | | | |
| IGJ | + | − | | | |
| CGB7 | + | + | | | |
| CSF3 | + | + | | | |
| VEGFB | + | − | | | |
| FGF17 | − | + | | | |
| CRP | + | − | | | |
| CKLFSF4 | + | − | | | |
| TNFSF13 | − | + | | | |
| AZU1 | + | − | | | |
| KLKL5 | + | + | | | |
| ELA3B | + | − | | | |
| ELA3B | − | + | | | |
| SPARC | + | − | | | |
| APOF | + | + | | | |
| APOF | + | + | | | |
| APOF | + | − | | | |
| APOF | + | + | | | |
| IL12B | − | + | | | |
| CD86 | + | − | | | |
| OPTC | + | + | | | |
| SFRP4 | + | + | | | |
| CD5L | − | + | | | |
| WNT11 | − | + | | | |
| GIP | + | − | | | |
| WNT2 | + | + | | | |
| ANGPTL4 | + | + | | | |
| VEGFA | + | + | | | |
| LFNG | + | + | | | |
| IL13RA2 | − | + | | | |
| PGC | − | + | | | |
| BMP15 | + | − | | | |
| GDF11 | − | + | | | |
| INHBB | − | + | | | |
| RHCE | + | − | | | |
| INHBA | + | − | | | |
| GLA | + | − | | | |
| EFEMP2 | + | − | | | |
| EFEMP2 | + | − | | | |
| TNFRSF1A | + | − | | | |
| CPN1 | + | − | | | |
| CPN1 | − | + | | | |
| PNLIPRP1 | + | + | | | |
| PNLIPRP1 | + | + | | | |
| GC | − | + | + | | |
| MMP28 | + | − | | | |
| MMP25 | + | − | + | | |
| NMB | − | + | | | |
| VGF | + | + | | | |
| PCSK9 | + | + | + | | |
| VCAM1 | − | + | | | |
| LOXL3 | − | + | | | |
| COMP | + | + | | + | |
| COMP | + | − | | | |
| SEMA3A | + | − | | | |
| FURIN | + | − | | | |
| FURIN | + | + | | | |
| NLGN1 | + | − | | | |
| NLGN3 | + | − | | | |
| POSTN | − | + | | | |
| MATN2 | + | + | | + | |
| BMP1 | + | − | | + | |
| 97 | + | − | | | |

TABLE 2B

| | | | | |
|---|---|---|---|---|
| Gene | | SEQ | | SEQ |
| Name | Nucleotide Sequence | ID NO: | Amino Acid Sequence | ID NO: |

| | | | | |
|---|---|---|---|---|
| PF4V1 | CCCAGGCACATCACCAGCCTGGAGGTGATCAAGGCCGGACCC | 48 | PRHITSLEVIKAGPHCPTAQLIATLKNGRKI | 49 |
| | CACTGCCCCACTGCCCAACTCATAGCCACGCTGAAGAATGGG | | CLDLQALLYKKIIKEHLES | |
| | AGGAAAATTTGCTTGGATCTGCAAGCCCTGCTGTACAAGAAA | | | |
| | ATCATTAAGGAACATTTGGAGAGT | | | |
| CCK | ATCCAGCAGGCCCGGAAAGCTCCTTCTGGACGAATGTCCATC | 50 | IQQARKAPSGRMSIVKNLQNLDPSHRISDRD | 51 |
| | GTTAAGAACCTGCAGAACCTGGACCCCAGCCACAGGATAAGT | | YMGWMDFGRRSAEEYEYPS | |
| | GACCGGGACTACATGGGCTGGATGGATTTTGGCCGTCGCAGT | | | |
| | GCCGAGGAGTATGAGTACCCCTCC | | | |
| NPPA | CCTCCCTGGACCGGGGAAGTCAGCCCAGCCCAGAGAGATGGA | 52 | PPWTGEVSPAQRDGGALGRGPWDSSDRSALL | 53 |
| | GGTGCCCTCGGGCGGGGCCCCTGGGACTCCTCTGATCGATCT | | KSKLRALLTAPRSLRRSSC | |
| | GCCCTCCTAAAAAGCAAGCTGAGGGCGCTGCTCACTGCCCCT | | | |
| | CGGAGCCTGCGGAGATCCAGCTGC | | | |
| IGJ | ATGAAGAACCATTTGCTTTTCTGGGGAGTCCTGGCGGTTTTT | 54 | MKNHLLFWGVLAVFIKAVHVKAQEDERIVLV | 55 |
| | ATTAAGGCTGTTCATGTGAAAGCCCAAGAAGATGAAAGGATT | | DNKCKCARITSRIIRSSED | |
| | GTTCTTGTTGACAACAAATGTAAGTGTGCCCGGATTACTTCC | | | |
| | AGGATCATCCGTTCTTCCGAAGAT | | | |
| CGB7 | GATGTGCGCTTCGAGTCCATCCGGCTCCCTGGCTGCCCGCGC | 56 | DVRFESIRLPGCPRGVNPVVSYAVALSCQCA | 57 |
| | GGCGTGAACCCCGTGGTCTCCTACGCCGTGGCTCTCAGCTGT | | LCRRSTTDCGGPKDHPLTC | |
| | CAATGTGCACTCTGCCGCCGCAGCACCACTGACTGCGGGGGT | | | |
| | CCCAAGGACCACCCCTTGACCTGT | | | |
| CSF3 | GTGCTGCTCGGACACTCTCTGGGCATCCCCTGGGCTCCCCTG | 58 | VLLGHSLGIPWAPLSSCPSQALQLAGCLSQL | 59 |
| | AGCAGCTGCCCCAGCCAGGCCCTGCAGCTGGCAGGCTGCTTG | | HSGLFLYQGLLQALEGISP | |
| | AGCCAACTCCATAGCGGCCTTTTCCTCTACCAGGGGCTCCTG | | | |
| | CAGGCCCTGGAAGGGATCTCCCCC | | | |
| VEGFB | GAGGTGGTGGTGCCCTTGACTGTGGAGCTCATGGGCACCGTG | 60 | EVVVPLTVELMGTVAKQLVPSCVTVQRCGGC | 61 |
| | GCCAAACAGCTGGTGCCCAGCTGCGTGACTGTGCAGCGCTGT | | CPDDGLECVPTGQHQVRMQ | |
| | GGTGGCTGCTGCCCTGACGATGGCCTGGAGTGTGTGCCCACT | | | |
| | GGGCAGCACCAAGTCCGGATGCAG | | | |
| FGF17 | AACAAGTTTGCCAAGCTCATAGTGGAGACGGACACGTTTGGC | 62 | NKFAKLIVETDTFGSRVRIKGAESEKYICMN | 63 |
| | AGCCGGGTTCGCATCAAAGGGGCTGAGAGTGAGAAGTACATC | | KRGKLIGKPSGKSKDCVFT | |
| | TGTATGAACAAGAGGGGCAAGCTCATCGGGAAGCCCAGCGGG | | | |
| | AAGAGCAAAGACTGCGTGTTCACG | | | |
| CRP | AAGGGATACACTGTGGGGGCAGAAGCAAGCATCATCTTGGGG | 64 | KGYTVGAEASIILGQEQDSFGGNFEGSQSLV | 65 |
| | CAGGAGCAGGATTCCTTCGGTGGGAACTTTGAAGGAAGCCAG | | GDIGNVNMWDFVLSPDEIN | |
| | TCCCTGGTGGGAGACATTGGAAATGTGAACATGTGGGACTTT | | | |
| | GTGCTGTCACCAGATGAGATTAAC | | | |
| CKLFSF4 | ATTGCTGCCGTGATATTTGGCTTCTTGGCGACTGCGGCATAT | 66 | IAAVIFGFLATAAYAVNTFLAVQKWRVSVRQ | 67 |
| | GCAGTGAACACATTCCTGGCAGTGCAGAAATGGAGAGTCAGC | | QSTNDYIRARTESRDVDSR | |
| | GTCCGCCAGCAGAGCACCAATGACTACATCCGAGCCCGCACG | | | |
| | GAGTCCAGGGATGTGGACAGTCGC | | | |
| TNFSF13 | CAACAAACAGAGCTGCAGAGCCTCAGGAGAGAGGTGAGCCGG | 68 | QQTELQSLRREVSRLQGTGGPSQNGEGYPWQ | 69 |
| | CTGCAGGGGACAGGAGGCCCCTCCCAGAATGGGGAAGGGTAT | | SLPEQSSDALEAWENGERS | |
| | CCCTGGCAGAGTCTCCCGGAGCAGAGTTCCGATGCCCTGGAA | | | |
| | GCCTGGGAGAATGGGGAGAGATCC | | | |
| AZU1 | AGCATGAGCGAGAATGGCTACGACCCCCAGCAGAACCTGAAC | 70 | SMSENGYDPQQNLNDLMLLQLDREANLTSSV | 71 |
| | GACCTGATGCTGCTTCAGCTGGACCGTGAGGCCAACCTCACC | | TILPLPLQNATVEAGTRCQ | |
| | AGCAGCGTGACGATACTGCCACTGCCTCTGCAGAACGCCACG | | | |
| | GTGGAAGCCGGCACCAGATGCCAG | | | |
| KLKL5 | GGGGGCCCCCTGGTGTGTGGGGGAGTCCTTCAAGGTCTGGTG | 72 | GGPLVCGGVLQGLVSWGSVGPCGQDGIPGVY | 73 |
| | TCCTGGGGGTCTGTGGGGCCCTGTGGACAAGATGGCATCCCT | | TYICNSTLVGLGTSWNFNS | |
| | GGAGTCTACACCTATATTTGCAACTCCACTCTTGTTGGCCTG | | | |
| | GGAACTTCTTGGAACTTTAACTCC | | | |
| ELA3B | CTTCCCAACGAGACACCCTGCTACATCACCGGCTGGGGCCGT | 74 | LPNETPCYITGWGRLYTNGPLPDKLQEALLP | 75 |
| | CTCTATACCAACGGGCCACTCCCAGACAAGCTGCAGGAGGCC | | VVDYEHCSRWNWWGSSVKK | |
| | CTGCTGCCGGTGGTGGACTATGAACACTGCTCCAGGTGGAAC | | | |
| | TGGTGGGGTTCCTCCGTGAAAAAG | | | |
| ELA3B | TGGAACTGGTGGGGTTCCTCCGTGAAAAAGACCATGGTGTGT | 76 | WNWWGSSVKKTMVCAGGDIRSGCNGDSGGPL | 77 |
| | GCTGGAGGGGACATCCGCTCCGGCTGCAATGGTGACTCTGGA | | NCPTEDGGWQVHGVTSFVS | |
| | GGACCCCTCAACTGCCCCACAGAGGATGGTGGCTGGCAGGTC | | | |
| | CATGGCGTGACCAGCTTTGTTTCT | | | |
| SPARC | GTGGAAGAAACTGTGGCAGAGGTGACTGAGGTATCTGTGGGA | 78 | VEETVAEVTEVSVGANPVQVEVGEFDDGAEE | 79 |
| | GCTAATCCTGTCCAGGTGGAAGTAGGAGAATTTGATGATGGT | | TEEEVVAENPCQNHHCKHG | |
| | GCAGAGGAAACCGAAGAGGAGGTGGTGGCGGAAAATCCCTGC | | | |
| | CAGAACCACCACTGCAAACACGGC | | | |
| APOF | CAGGTCCTCATCCAGCATCTTCGAGGGCTCCAGAAAGGCAGA | 80 | QVLIQHLRGLQKGRSTERNVSVEALASALQL | 81 |
| | AGCACAGAGAGGAACGTGTCAGTGGAAGCCCTGGCCTCTGCT | | LAREQQSTGRVGRSLPTED | |
| | CTGCAGCTGTTAGCCAGGGAGCAGCAAAGCACAGGAAGGGTC | | | |
| | GGGCGCTCCCTCCCGACAGAGGAC | | | |
| APOF | CAGAAAGGCAGAAGCACAGAGAGGAACGTGTCAGTGGAAGCC | 82 | QKGRSTERNVSVEALASALQLLAREQQSTGR | 83 |
| | CTGGCCTCTGCTCTGCAGCTGTTAGCCAGGGAGCAGCAAAGC | | VGRSLPTEDCENEKEQAVH | |
| | ACAGGAAGGGTCGGGCGCTCCCTCCCGACAGAGGACTGTGAG | | | |
| | AATGAGAAGGAGCAAGCTGTGCAC | | | |
| APOF | TCAGTGGAAGCCCTGGCCTCTGCTCTGCAGCTGTTAGCCAGG | 84 | SVEALASALQLLAREQQSTGRVGRSLPTEDC | 85 |
| | GAGCAGCAAAGCACAGGAAGGGTCGGGCGCTCCCTCCCGACA | | ENEKEQAVHNVVQLLPGVG | |
| | GAGGACTGTGAGAATGAGAAGGAGCAAGCTGTGCACAATGTA | | | |
| | GTCCAGCTGCTGCCAGGAGTGGGA | | | |
| APOF | CTGTTAGCCAGGGAGCAGCAAAGCACAGGAAGGGTCGGGCGC | 86 | LLAREQQSTGRVGRSLPTEDCENEKEQAVHN | 87 |
| | TCCCTCCCGACAGAGGACTGTGAGAATGAGAAGGAGCAAGCT | | VVQLLPGVGTFYNLGTALY | |
| | GTGCACAATGTAGTCCAGCTGCTGCCAGGAGTGGGAACCTTC | | | |
| | TACAACCTGGGCACAGCTTTGTAT | | | |
| IL12B | GACATCATCAAACCTGACCCACCCAAGAACTTGCAGCTGAAG | 88 | DIIKPDPPKNLQLKPLKNSRQVEVSWEYPDT | 89 |
| | CCATTAAAGAATTCTCGGCAGGTGGAGGTCAGCTGGGAGTAC | | WSTPHSYFSLTFCVQVQGK | |
| | CCTGACACCTGGAGTACTCCACATTCCTACTTCTCCCTGACA | | | |
| | TTCTGCGTTCAGGTCCAGGGCAAG | | | |
| CD86 | ATCAGCTTGTCTGTTTCATTCCCTGATGTTACGAGCAATATG | 90 | ISLSVSFPDVTSNMTIFCILETDKTRLLSSP | 91 |
| | ACCATCTTCTGTATTCTGGAAACTGACAAGACGCGGCTTTTA | | FSIELEDPQPPPDHIPWIT | |
| | TCTTCACCTTTCTCTATAGAGCTTGAGGACCCTCAGCCTCCC | | | |
| | CCAGACCACATTCCTTGGATTACA | | | |
| OPTC | TTCCTTTACCTGTCAGACAACCTGCTGGATTCTATCCCGGGG | 92 | FLYLSDNLLDSIPGPLPLSLRSVHLQNNLIE | 93 |
| | CCTTTGCCCCTGAGCCTGCGCTCTGTACACCTGCAGAATAAC | | TMQRDVFCDPEEHKHTRRQ | |
| | CTGATAGAGACCATGCAGAGAGACGTATTCTGTGACCCCGAG | | | |
| | GAGCACAAACACACCCGCAGGCAG | | | |
| SFRP4 | GCCGTGCTGCGCTTCTTCCTCTGTGCCATGTACGCGCCCATT | 94 | AVLRFFLCAMYAPICTLEFLHDPIKPCKSVC | 95 |
| | TGCACCCTGGAGTTCCTGCACGACCCTATCAAGCCGTGCAAG | | QRARDDCEPLMKMYNHSWP | |
| | TCGGTGTGCCAACGCGCGCGCGACGACTGCGAGCCCCTCATG | | | |
| | AAGATGTACAACCACAGCTGGCCC | | | |
| CD5L | GATACATTGGCTCAGTGTGAGCAAGAAGAAGTTTATGATTGT | 96 | DTLAQCEQEEVYDCSHDEDAGASCENPESSF | 97 |
| | TCACATGATGAAGATGCTGGGGCATCGTGTGAGAACCCAGAG | | SPVPEGVRLADGPGHCKGR | |
| | AGCTCTTTCTCCCCAGTCCCAGAGGGTGTCAGGCTGGCTGAC | | | |
| | GGCCCTGGGCATTGCAAGGGACGC | | | |
| WNT11 | CTACACAACAGTGAAGTGGGGAGACAGGCTCTGCGCGCCTCT | 98 | LHNSEVGRQALRASLEMKCKCHGVSGSCSIR | 99 |
| | CTGGAAATGAAGTGTAAGTGCCATGGGGTGTCTGGCTCCTGC | | TCWKGLQELQDVAADLKTR | |
| | TCCATCCGCACCTGCTGGAAGGGGCTGCAGGAGCTGCAGGAT | | | |
| | GTGGCTGCTGACCTCAAGACCCGA | | | |
| GIP | TACACAGGGGCCAACAAATATGATGAGGCAGCCAGCTACATC | 100 | YTGANKYDEAASYIQSKFEDLNKRKDTKEIY | 101 |
| | CAGAGTAAGTTTGAGGACCTGAATAAGCGCAAAGACACCAAG | | THFTCATDTKNVQFVFDAV | |
| | GAGATCTACACGCACTTCACGTGCGCCACCGACACCAAGAAC | | | |
| | GTGCAGTTCGTGTTTGACGCCGTC | | | |
| WNT2 | AAGAAGCCAACGAAAAATGACCTCGTGTATTTTGAGAATTCT | 102 | KKPTKNDLVYFENSPDYCIRDREAGSLGTAG | 103 |
| | CCAGACTACTGTATCAGGGACCGAGAGGCAGGCTCCCTGGGT | | RVCNLTSRGMDSCEVMCCG | |
| | ACAGCAGGCCGTGTGTGCAACCTGACTTCCCGGGGCATGGAC | | | |
| | AGCTGTGAAGTCATGTGCTGTGGG | | | |
| ANGPTL4 | CTGATGCTCTGCGCCGCCACCGCCGTGCTACTGAGCGCTCAG | 104 | LMLCAATAVLLSAQGGPVQSKSPRFASWDEM | 105 |
| | GGCGGACCCGTGCAGTCCAAGTCGCCGCGCTTTGCGTCCTGG | | NVLAHGLLQLGQGLREHAE | |
| | GACGAGATGAATGTCCTGGCGCACGGACTCCTGCAGCTCGGC | | | |
| | CAGGGGCTGCGCGAACACGCGGAG | | | |
| VEGFA | GCGGGGGAAGCCGAGCCGAGCGGAGCCGCGAGAAGTGCTAGC | 106 | AGEAEPSGAARSASSGREEPQPEEGEEEEEK | 107 |
| | TCGGGCCGGGAGGAGCCGCAGCCGGAGGAGGGGGAGGAGGAA | | EEERGPQWRLGARKPGSWT | |
| | GAAGAGAAGGAAGAGGAGAGGGGGCCGCAGTGGCGACTCGGC | | | |
| | GCTCGGAAGCCGGGCTCATGGACG | | | |
| LFNG | CTGGGTGTGCCCCTCATCCGCAGCGGCCTCTTCCACTCCCAC | 108 | LGVPLIRSGLFHSHLENLQQVPTSELHEQVT | 109 |
| | CTGGAGAACCTGCAGCAGGTGCCCACCTCGGAGCTCCACGAG | | LSYGMFENKRNAVHVKGPF | |
| | CAGGTGACGCTGAGCTACGGTATGTTTGAAAACAAGCGGAAC | | | |
| | GCCGTCCACGTGAAGGGGCCCTTC | | | |
| IL13RA2 | AGTTCCTGGGCAGAAACTACTTATTGGATATCACCACAAGGA | 110 | SSWAETTYWISPQGIPETKVQDMDCVYYNWQ | 111 |
| | ATTCCAGAAACTAAAGTTCAGGATATGGATTGCGTATATTAC | | YLLCSWKPGIGVLLDTNYN | |
| | AATTGGCAATATTTACTCTGTTCTTGGAAACCTGGCATAGGT | | | |
| | GTACTTCTTGATACCAATTACAAC | | | |
| PGC | CTCCAGCTCTTGGAGGCAGCAGTGGTCAAAGTGCCCCTGAAG | 112 | LQLLEAAVVKVPLKKFKSIRETMKEKGLLGE | 113 |
| | AAATTTAAGTCTATCCGTGAGACCATGAAGGAGAAGGGCTTG | | FLRTHKYDPAWKYRFGDLS | |
| | CTGGGGGAGTTCCTGAGGACCCACAAGTATGATCCTGCTTGG | | | |
| | AAGTACCGCTTTGGTGACCTCAGC | | | |
| BMP15 | TCAAAACATAGCGGGCCTGAAAATAACCAGTGTTCCCTCCAC | 114 | SKHSGPENNQCSLHPFQISFRQLGWDHWIIA | 115 |
| | CCTTTCCAAATCAGCTTCCGCCAGCTGGGTTGGGATCACTGG | | PPFYTPNYCKGTCLRVLRD | |
| | ATCATTGCTCCCCCTTTCTACACCCCAAACTACTGTAAAGGA | | | |
| | ACTTGTCTCCGAGTACTACGCGAT | | | |
| GDF11 | GTCACCTCCCTGGGGCCGGGAGCCGAGGGGCTGCATCCATTC | 116 | VTSLGPGAEGLHPFMELRVLENTKRSRRNLG | 117 |
| | ATGGAGCTTCGAGTCCTAGAGAACACAAAACGTTCCCGGCGG | | LDCDEHSSESRCCRYPLTV | |
| | AACCTGGGTCTGGACTGCGACGAGCACTCAAGCGAGTCCCGC | | | |
| | TGCTGCCGATATCCCCTCACAGTG | | | |
| INHBB | CACACGGCTGTGGTGAACCAGTACCGCATGCGGGGTCTGAAC | 118 | HTAVVNQYRMRGLNPGTVNSCCIPTKLSTMS | 119 |
| | CCCGGCACGGTGAACTCCTGCTGCATTCCCACCAAGCTGAGC | | MLYFDDEYNIVKRDVPNMI | |
| | ACCATGTCCATGCTGTACTTCGATGATGAGTACAACATCGTC | | | |
| | AAGCGGGACGTGCCCAACATGATT | | | |
| RHCE | ATCTTCAGCTTGCTGGGTCTGCTTGGAGAGATCACCTACATT | 120 | IFSLLGLLGEITYIVLLVLHTVWNGNGMIGF | 121 |
| | GTGCTGCTGGTGCTTCATACTGTCTGGAACGGCAATGGCATG | | QVLLSIGELSLAIVIALTS | |
| | ATTGGCTTCCAGGTCCTCCTCAGCATTGGGGAACTCAGCTTG | | | |
| | GCCATCGTGATAGCTCTCACGTCT | | | |
| INHBA | CTGGACCAGGGCAAGAGCTCCCTGGACGTTCGGATTGCCTGT | 122 | LDQGKSSLDVRIACEQCQESGASLVLLGKKK | 123 |
| | GAGCAGTGCCAGGAGAGTGGCGCCAGCTTGGTTCTCCTGGGC | | KKEEEGEGKKKGGGEGGAG | |
| | AAGAAGAAGAAGAAAGAAGAGGAGGGGGAAGGGAAAAAGAAG | | | |
| | GGCGGAGGTGAAGGTGGGGCAGGA | | | |
| GLA | GAGAGAATTGTTGATGTTGCTGGACCAGGGGGTTGGAATGAC | 124 | ERIVDVAGPGGWNDPDMLVIGNFGLSWNQQV | 125 |
| | CCAGATATGTTAGTGATTGGCAACTTTGGCCTCAGCTGGAAT | | TQMALWAIMAAPLFMSNDL | |
| | CAGCAAGTAACTCAGATGGCCCTCTGGGCTATCATGGCTGCT | | | |
| | CCTTTATTCATGTCTAATGACCTC | | | |
| EFEMP2 | GCCCCATGCGAGCAGCGCTGCTTCAACTCCTATGGGACCTTC | 126 | APCEQRCFNSYGTFLCRCHQGYELHRDGFSC | 127 |
| | CTGTGTCGCTGCCACCAGGGCTATGAGCTGCATCGGGATGGC | | SDIDECSYSSYLCQYRCIN | |
| | TTCTCCTGCAGTGATATTGATGAGTGTAGCTACTCCAGCTAC | | | |
| | CTCTGTCAGTACCGCTGCATCAAC | | | |
| EFEMP2 | TGCAGTGATATTGATGAGTGTAGCTACTCCAGCTACCTCTGT | 128 | CSDIDECSYSSYLCQYRCINEPGRFSCHCPQ | 129 |
| | CAGTACCGCTGCATCAACGAGCCAGGCCGTTTCTCCTGCCAC | | GYQLLATRLCQDIDECESG | |
| | TGCCCACAGGGTTACCAGCTGCTGGCCACACGCCTCTGCCAA | | | |
| | GACATTGATGAGTGTGAGTCTGGT | | | |
| TNFRSF1A | CAGAACGGGCGCTGCCTGCGCGAGGCGCAATACAGCATGCTG | 130 | QNGRCLREAQYSMLATWRRRTPRREATLELL | 131 |
| | GCGACCTGGAGGCGGCGCACGCCGCGGCGCGAGGCCACGCTG | | GRVLRDMDLLGCLEDIEEA | |
| | GAGCTGCTGGGACGCGTGCTCCGCGACATGGACCTGCTGGGC | | | |
| | TGCCTGGAGGACATCGAGGAGGCG | | | |
| CPN1 | TTGGGCCGCGAGCTGATGCTGCAGCTGTCGGAGTTTCTGTGC | 132 | LGRELMLQLSEFLCEEFRNRNQRIVQLIQDT | 133 |
| | GAGGAGTTCCGGAACAGGAACCAGCGCATCGTCCAGCTCATC | | RIHILPSMNPDGYEVAAAQ | |
| | CAGGACACGCGCATTCACATCCTGCCATCCATGAACCCCGAC | | | |
| | GGCTACGAGGTGGCTGCTGCCCAG | | | |
| CPN1 | TTCCAGAAGCTGGCCAAGGTCTACTCCTATGCACATGGATGG | 134 | FQKLAKVYSYAHGWMFQGWNCGDYFPDGITN | 135 |
| | ATGTTCCAAGGTTGGAACTGCGGAGATTACTTCCCAGATGGC | | GASWYSLSKGMQDFNYLHT | |
| | ATCACCAATGGGGCTTCCTGGTATTCTCTCAGCAAGGGAATG | | | |
| | CAAGACTTTAATTATCTCCATACC | | | |
| PNLIPRP1 | AGCCTGGGAGCCCACGTGGCTGGAGAGGCAGGAAGCAAGACT | 136 | SLGAHVAGEAGSKTPGLSRITGLDPVEASFE | 137 |
| | CCAGGCCTGAGCAGGATTACAGGGTTGGATCCTGTAGAAGCA | | STPEEVRLDPSDADFVDVI | |
| | AGTTTCGAGAGTACTCCTGAAGAGGTGCGACTTGATCCCTCT | | | |
| | GATGCTGACTTTGTTGATGTGATT | | | |
| PNLIPRP1 | GGAAGCAAGACTCCAGGCCTGAGCAGGATTACAGGGTTGGAT | 138 | GSKTPGLSRITGLDPVEASFESTPEEVRLDP | 139 |
| | CCTGTAGAAGCAAGTTTCGAGAGTACTCCTGAAGAGGTGCGA | | SDADFVDVIHTDAAPLIPF | |
| | CTTGATCCCTCTGATGCTGACTTTGTTGATGTGATTCACACG | | | |
| | GATGCAGCTCCCCTGATCCCATTC | | | |
| GC | AAATTTCCCAGTGGCACGTTTGAACAGGTCAGCCAACTTGTG | 140 | KFPSGTFEQVSQLVKEVVSLTEACCAEGADP | 141 |
| | AAGGAAGTTGTCTCCTTGACCGAAGCCTGCTGTGCGGAAGGG | | DCYDTRTSALSAKSCESNS | |
| | GCTGACCCTGACTGCTATGACACCAGGACCTCAGCACTGTCT | | | |
| | GCCAAGTCCTGTGAAAGTAATTCT | | | |
| MMP28 | TACTACAAGAGGCTGGGCCGCGACGCGCTGCTCAGCTGGGAC | 142 | YYKRLGRDALLSWDDVLAVQSLYGKPLGGSV | 143 |
| | GACGTGCTGGCCGTGCAGAGCCTGTATGGGAAGCCCCTAGGG | | AVQLPGKLFTDFETWDSYS | |
| | GGCTCAGTGGCCGTCCAGCTCCCAGGAAAGCTGTTCACTGAC | | | |
| | TTTGAGACCTGGGACTCCTACAGC | | | |
| MMP25 | ATGCGGCTGCGGCTCCGGCTTCTGGCGCTGCTGCTTCTGCTG | 144 | MRLRLRLLALLLLLLAPPARAPKPSAQDVSL | 145 |
| | CTGGCACCGCCCGCGCGCGCCCCGAAGCCCTCGGCGCAGGAC | | GVDWLTRYGYLPPPHPAQA | |
| | GTGAGCCTGGGCGTGGACTGGCTGACTCGCTATGGTTACCTG | | | |
| | CCGCCACCCCACCCTGCCCAGGCC | | | |
| NMB | TCTGGGACGTACTGTGTGAACCTCACCCTGGGGGATGACACA | 146 | SGTYCVNLTLGDDTSLALTSTLISVPDRDPA | 147 |
| | AGCCTGGCTCTCACGAGCACCCTGATTTCTGTTCCTGACAGA | | SPLRMANSALISVGCLAIF | |
| | GACCCAGCCTCGCCTTTAAGGATGGCAAACAGTGCCCTGATC | | | |
| | TCCGTTGGCTGCTTGGCCATATTT | | | |
| VGF | AACGCGCTCCTGTTCGCGGAGGAGGAGGACGGGGAAGCCGGC | 148 | NALLFAEEEDGEAGAEDKRSQEETPGHRRKE | 149 |
| | GCCGAGGACAAGCGCTCCCAGGAGGAGACGCCGGGCCACCGG | | AEGTEEGGEEEDDEEMDPQ | |
| | CGGAAGGAGGCCGAGGGGACAGAGGAGGGCGGGGAGGAGGAG | | | |
| | GACGACGAGGAGATGGATCCGCAG | | | |
| PCSK9 | CTGCTCCTGGGTCCCGCGGGCGCCCGTGCGCAGGAGGACGAG | 150 | LLLGPAGARAQEDEDGDYEELVLALRSEEDG | 151 |
| | GACGGCGACTACGAGGAGCTGGTGCTAGCCTTGCGTTCCGAG | | LAEAPEHGTTATFHRCAKD | |
| | GAGGACGGCCTGGCCGAAGCACCCGAGCACGGAACCACAGCC | | | |
| | ACCTTCCACCGCTGCGCCAAGGAT | | | |
| VCAM1 | CACTCTTACCTGTGCACAGCAACTTGTGAATCTAGGAAATTG | 152 | HSYLCTATCESRKLEKGIQVEIYSFPKDPEI | 153 |
| | GAAAAAGGAATCCAGGTGGAGATCTACTCTTTTCCTAAGGAT | | HLSGPLEAGKPITVKCSVA | |
| | CCAGAGATTCATTTGAGTGGCCCTCTGGAGGCTGGGAAGCCG | | | |
| | ATCACAGTCAAGTGTTCAGTTGCT | | | |
| LOXL3 | AACAGTGACTGTACGCACGATGAGGATGCTGGGGTCATCTGC | 154 | NSDCTHDEDAGVICKDQRLPGFSDSNVIEVE | 155 |
| | AAAGACCAGCGCCTCCCTGGCTTCTCGGACTCCAATGTCATT | | HHLQVEEVRIRPAVGWGRR | |
| | GAGGTAGAGCATCACCTGCAAGTGGAGGAGGTGCGAATTCGA | | | |
| | CCCGCCGTTGGGTGGGGCAGACGA | | | |
| COMP | GACAGCGATCAAGACCAGGATGGAGACGGACATCAGGACTCT | 156 | DSDQDQDGDGHQDSRDNCPTVPNSAQEDSDH | 157 |
| | CGGGACAACTGTCCCACGGTGCCTAACAGTGCCCAGGAGGAC | | DGQGDACDDDDDNDGVPDS | |
| | TCAGACCACGATGGCCAGGGTGATGCCTGCGACGACGACGAC | | | |
| | GACAATGACGGAGTCCCTGACAGT | | | |
| COMP | CATCAGGACTCTCGGGACAACTGTCCCACGGTGCCTAACAGT | 158 | HQDSRDNCPTVPNSAQEDSDHDGQGDACDDD | 159 |
| | GCCCAGGAGGACTCAGACCACGATGGCCAGGGTGATGCCTGC | | DDNDGVPDSRDNCRLVPNP | |
| | GACGACGACGACGACAATGACGGAGTCCCTGACAGTCGGGAC | | | |
| | AACTGCCGCCTGGTGCCTAACCCC | | | |
| SEMA3A | GGAAGAGTCCCCTATCCACGGCCAGGAACTTGTCCCAGCAAA | 160 | GRVPYPRPGTCPSKTFGGFDSTKDLPDDVIT | 161 |
| | ACATTTGGTGGTTTTGACTCTACAAAGGACCTTCCTGATGAT | | FARSHPAMYNPVFPMNNRP | |
| | GTTATAACCTTTGCAAGAAGTCATCCAGCCATGTACAATCCA | | | |
| | GTGTTTCCTATGAACAATCGCCCA | | | |
| FURIN | GGCTACACAGGGCACGGCATTGTGGTCTCCATTCTGGACGAT | 162 | GYTGHGIVVSILDDGIEKNHPDLAGNYDPGA | 163 |
| | GGCATCGAGAAGAACCACCCGGACTTGGCAGGCAATTATGAT | | SFDVNDQDPDPQPRYTQMN | |
| | CCTGGGGCCAGTTTTGATGTCAATGACCAGGACCCTGACCCC | | | |
| | CAGCCTCGGTACACACAGATGAAT | | | |
| FURIN | AATGACGTGGAGACCATCCGGGCCAGCGTCTGCGCCCCCTGC | 164 | NDVETIRASVCAPCHASCATCQGPALTDCLS | 165 |
| | CACGCCTCATGTGCCACATGCCAGGGGCCGGCCCTGACAGAC | | CPSHASLDPVEQTCSRQSQ | |
| | TGCCTCAGCTGCCCCAGCCACGCCTCCTTGGACCCTGTGGAG | | | |
| | CAGACTTGCTCCCGGCAAAGCCAG | | | |
| NLGN1 | AATGAAATTTTGGGGCCTGTTATTCAATTTCTTGGGGTTCCA | 166 | NEILGPVIQFLGVPYAAPPTGERRFQPPEPP | 167 |
| | TATGCAGCCCCACCAACAGGGGAACGTCGTTTTCAGCCTCCA | | SPWSDIRNATQFAPVCPQN | |
| | GAACCACCATCTCCCTGGTCAGATATCAGAAATGCCACTCAA | | | |
| | TTTGCTCCTGTGTGTCCCCAGAAT | | | |
| NLGN3 | GTGGCCTGGTCCAAATACAATCCCCGAGACCAGCTCTACCTT | 168 | VAWSKYNPRDQLYLHIGLKPRVRDHYRATKV | 169 |
| | CACATCGGGCTGAAACCAAGGGTCCGAGATCATTACCGGGCC | | AFWKHLVPHLYNLHDMFHY | |
| | ACTAAGGTGGCCTTTTGGAAACATCTGGTGCCCCACCTATAC | | | |
| | AACCTGCATGACATGTTCCACTAT | | | |
| POSTN | AAGAACTGGTATAAAAAGTCCATCTGTGGACAGAAAACGACT | 170 | KNWYKKSICGQKTTVLYECCPGYMRMEGMKG | 171 |
| | GTTTTATATGAATGTTGCCCTGGTTATATGAGAATGGAAGGA | | CPAVLPIDHVYGTLGIVGA | |
| | ATGAAAGGCTGCCCAGCAGTTTTGCCCATTGACCATGTTTAT | | | |
| | GGCACTCTGGGCATCGTGGGAGCC | | | |
| MATN2 | CTGGCTGAGGATGGGAAGAGGTGTGTGGCTGTGGACTACTGT | 172 | LAEDGKRCVAVDYCASENHGCEHECVNADGS | 173 |
| | GCCTCAGAAAACCACGGATGTGAACATGAGTGTGTAAATGCT | | YLCQCHEGFALNPDKKTCT | |
| | GATGGCTCCTACCTTTGCCAGTGCCATGAAGGATTTGCTCTT | | | |
| | AACCCAGATAAAAAAACGTGCACA | | | |
| BMP1 | AAGATGGAGCCTCAGGAGGTGGAGTCCCTGGGGGAGACCTAT | 174 | KMEPQEVESLGETYDFDSIMHYARNTFSRGI | 175 |
| | GACTTCGACAGCATCATGCATTACGCTCGGAACACATTCTCC | | FLDTIVPKYEVNGVKPPIG | |
| | AGGGGCATCTTCCTGGATACCATTGTCCCCAAGTATGAGGTG | | | |
| | AACGGGGTGAAACCTCCCATTGGC | | | |
| 97 | GCGAAAATCGACGACAAAGGCGTTGTAACCAAGGGTGCTGAC | 176 | AKIDDKGVVTKGADVTDVKDPLATLDKALAQ | 177 |
| | GTTACTGACGTTAAAGATCCACTGGCTACCCTGGACAAAGCG | | VDGLRSSLGAVQNRFDSVI | |
| | CTGGCACAGGTTGACGGCCTGCGTTCTTCCCTGGGTGCGGTA | | | |
| | CAGAACCGTTTCGATTCTGTTATC | | | |

### Example 8

### Isolation of BASPs that Activate Other Signal Transduction Pathways

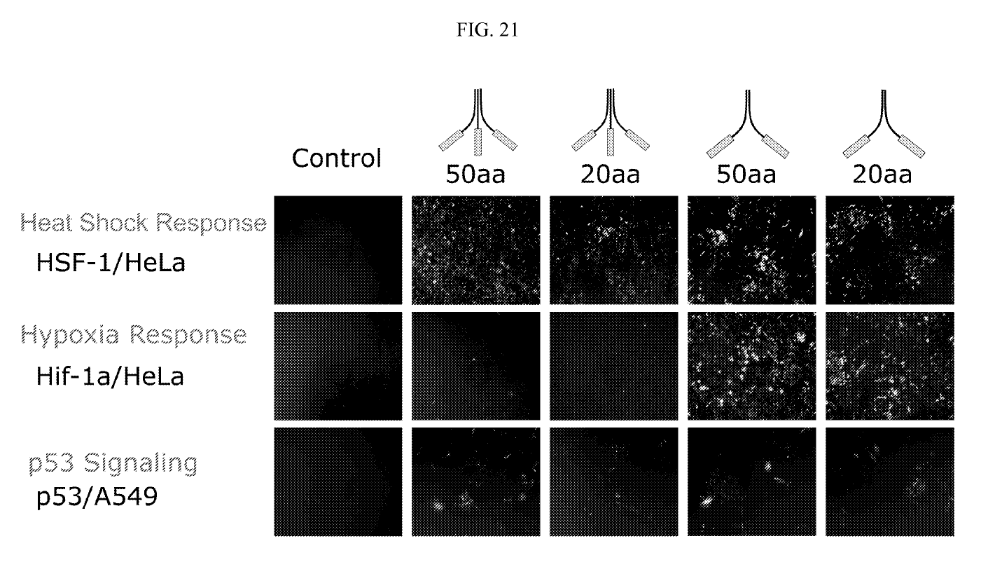

The experiments disclosed in Example 7 were substantially repeated using reporter cells having green fluorescent protein operatively linked to a variety of other promoters responsive to other stress responsive signal transduction pathways (including HSF-1, HIF1-alpha, and p53). The results of these screenings are shown in FIG. 21, which shows that positive results were obtained in all cases, illustrating the robustness of the screening methods of the invention. p53-activating BASPs caused growth arrest that resulted in large distinct GFP-expressing cells.

### Example 9

### Selection of Extracellular Peptides for 500K Secreted Peptide Libraries

In order to construct low-complexity (in comparison with random peptide) libraries enriched in potentially functional peptide ligands targeting cell surface receptors, a set of all known secreted, extracellular, and cell surface mammalian (human, mouse, and rat) proteins (roughly 4000 gene loci), are selected and then complemented with a set of extracellular proteins from other proteins of eukaryotic, prokaryotic, and viral origin that may regulate cell signaling. In particular, these include all membrane-bound, extracellular, and secreted proteins from pathogenic and symbiotic organisms, which frequently regulate host cell signaling. Based on the NCBI GenBank (RefSeq) and the Entrez Protein Database analysis using MeSH term key words, inter alia, for cytokine, chemokine, growth factor, receptor (extracellular domains), cell surface, extracellular, cell-cell communication, approximately 25,000 extracellular target proteins are expected to be selected. In order to select this comprehensive set of extracellular and membrane proteins, computational prediction and semantic analysis tools are applied as discussed herein. It is now well understood that proteins are often composed of multiple domains acting in concert. Since these domains are often modular, proteins can be dissected into their smallest functional motifs. It is commonly understood that these evolutionarily conserved domains (30aa-300aa in length) comprise functional motifs that possess binding, activation, repression, catalytic, and active substrate sites, which may modulate cell signaling through cell surface receptors and other mechanisms. Using the Conservative Domain Database (CDD) (Marchler-Bauer et al., 2009), and multiple sequence alignment algorithms available at the CDD and previously developed (Basu et al., 2008, Genome Res. 18: 449-61; Karey et al., 2002, Evol. Biol. 2: 18-25; Anantharaman et al., 2003), a set of evolutionarily conserved protein domains (estimated 100,000) in target extracellular proteins are identified. Considering the limitations in oligonucleotide chemistry, oligonucleotide templates can currently be synthesized for full-length “small” domains of less than 60aa (about 30% of all domains). For large domains (60aa-300aa), and even for some small domains with a modular structure, a redundant set of 2-20 conservative subdomains (15aa-60aa) is selected that often form stable folds and have specific biological functions. Insoluble peptide sequences and those that may induce significant immunogenicity due to the presence of MHC-II epitopes are excluded from the complete set of domain/subdomains (Chirino et al., 2004, Drug. Discov. Today 9: 82-90). All prokaryotic and viral sequences are codon-optimized for expression in mammalian cells. From the entire set of selected domain/subdomain sequences, about 500,000 template oligonucleotides are designed.

### Example 10

### Construction and Experimental Validation of 500K Extracellular Peptide Libraries

Using the protocols set forth herein, a pool of about 500,000 oligonucleotides encoding extracellular domain/subdomain peptides were synthesized on the surface of custom microarrays (two arrays with 244,000 oligos each). These oligonucleotides were then amplified with primers complementary to common flanking sequences, the fragment digested with BbsI, and cloned into BbsI sites in the set of lentiviral vectors as described and illustrated herein. 5×10⁵ peptide cassettes were cloned into scaffold vector designs that demonstrate the optimum performance in the validation studies (as discussed herein). Additional peptide libraries were also constructed in lentiviral vectors to permit expression of peptides under the control of a tet-regulated CMV promoter in order to extend application of the 500K peptide libraries to screening for cytotoxic peptides.

### Example 11

### Functional HTS for Cytotoxic or Cytostatic BASPs in an NCI-60 Cancer Cell Line Panel

Fourteen publically available databases (including Peptide Database, Cancer Immunity; PepBank, Massachusetts General Hospital, Harvard University; Antimicrobial Peptide Database; Bioactive Polypeptide Database; domino—domain peptide interaction; PeptideDB bioactive peptide database; Antimicrobial Peptide Database, Eppley Cancer Center, University of Nebraska Medical Center; Peptide Station; PhytAMP; Eurkeyotic Linear Motif resource for Functional Sites in Proteins; 3DID—3D interacting domains; Conserved Domains, National Center for Biotechnology Information (NCBI); and PDZBase, Institute for Computational

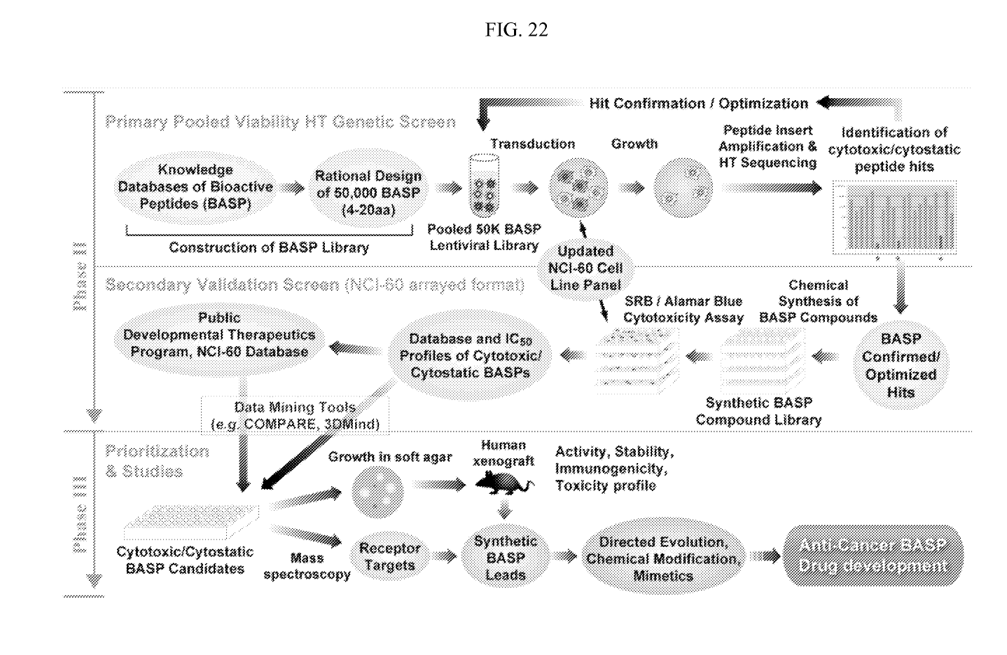

Biomedicine, Weill Medical College of Cornell University) and manually curated lists of bioactive peptides with a variety of anticancer, cytotoxic, antimicrobial, cardiovascular, apoptotic, angiogenic, immunomodulatory, and other activities are used for the design of approximately 50,000 peptides of 4-20 amino acid residues in length that could putatively modulate cellular responses by interacting with cell surface receptors (FIG. 22). The peptides target approximately 40,000 known natural and artificially-derived peptides (4-50 amino acids in length).

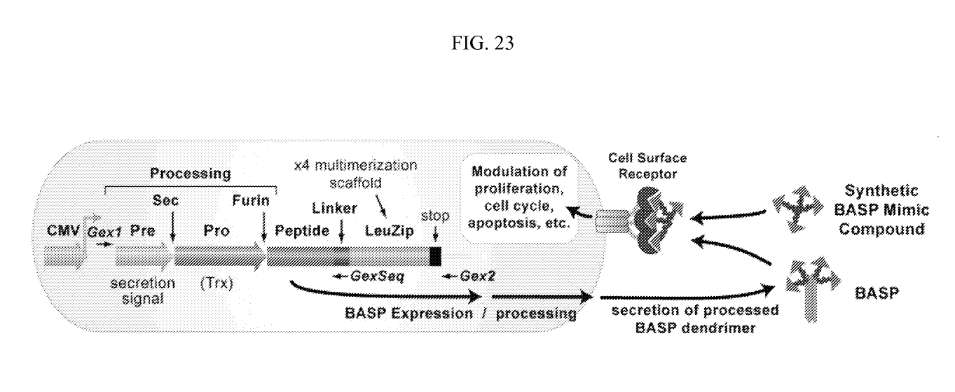

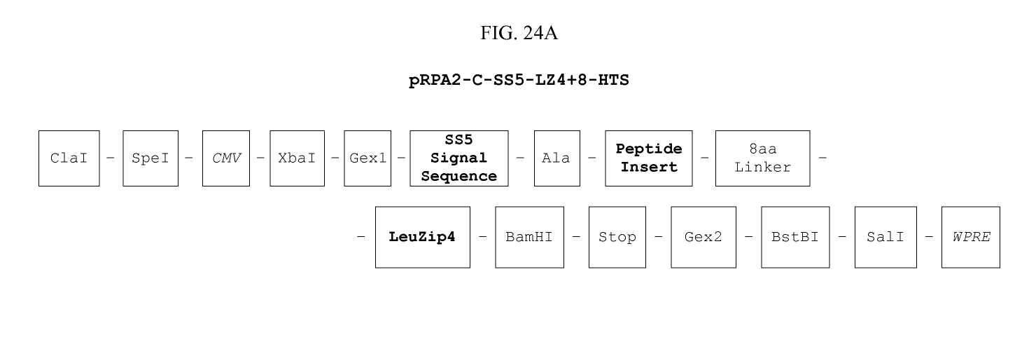

The 50K BASP library is constructed using HT oligonucleotide synthesis on the surface of microarrays (Agilent, Santa Clara, Calif.) as described herein, and the peptide cassettes are cloned such that they are under the control of the CMV promoter in a lentiviral vector that expresses secreted pre-pro-peptides in the tetrameric LeuZip scaffold. This approach has been successfully used in the development of TRAIL agonists (Li et al., 2006). The pre-pro-peptide design mimics the structure of most secreted precursors of cytokines and hormones. The secretion of mature, branched peptides is based on conventional processing (removal of the pre signal sequence) and folding (tetramer formation) in the ER followed by removal of the secretion targeting and protection pro moiety in the late Golgi by constitutive site-specific proteases of the furin family (FIG. 23).

A set of 20 of the most informative and well-characterized cancer cell lines for each of eleven cancer types is used for a primary screen of the 50K BASP library (Table 3; double-underlining indicates minimum balanced set of 20 most informative, validated cell lines for primary and confirmation screens with pooled BASP libraries). These cell lines have been successfully used in the NCI-60 panel (Skerra, 2007; Binz et al., 2005), J-39 panel (Yamori et al., 2003, Cancer Chemother. Pharmacol. 52: S74-79), and several large-scale RNAi viability screens (Luo et al., 2008, Proc. Natl. Aced. Sci. U.S.A. 105: 20380-85; Scholl et al., 2009, Cell 137: 8210-34; Luo et al., 2009, Cell 137: 835-48).

TABLE 3

| Cancer Type | Cell Line |
|---|---|
| Hematopoietic | HL-60, K-562, Jurkat, U937 |
| Lung (non-small) | NCI-H460, A549, NCI-H226, NCI-H23, NCI-H522, |
| | H1299 |
| Lung (small) | DMS114 |
| Colon | HCC-2998, HCT-116, HCT-15, HT-29, KM-12, |
| | DLD-1, SW480 |
| CNS | SF-266, U87-MG, SF-295, SF-539, SNB-75, |
| | SNB-78, SK-N-BEN2(c), Rh18 |
| Melanoma | SK-MEL-5, SK-MEL-28 |
| Ovarian | SK-OV-3, OVCAR-3, OVCAR-4, OVCAR-8 |
| Renal | 786-O, ACHN, RXF-631, HEK293 |
| Prostate | PC-3, DU-145, LnCap, CWR22 |
| Breast | MCF7, MDA-MB-231, MDA-MB453, MDA-MB-468, |
| | HS578T, T47D, HMEC |
| Pancreas | PANC-1, PaCa2, BxPC3 |
| Liver | HepG2, Hep3B |
| Connective | Saos-2, HT1080, U20S |
| Tissue/Bone | |
| Stomach | ST-4, MKN-1 |
| Skin | A431, A253, BCC-1/KMB |
| Head/Neck | SCC25 |

To select the 20 best cell lines, optimize protocols for cell growth, and conduct large-scale viability screens, a set of approximately 10 positive control cytotoxic dendrimeric peptide constructs in the pBASP vector are prepared. The control cytotoxic dendrimeric peptide constructs are prepared from sequences that have been previously described to reduce the viability of cancer cells through the activation of death receptors such as DRS, CD40, Erb1, the TNF family, VEGF, and ErbB2 (Orzaez et al., 2009; Li et al., 2006; Fatah et al., 2006; Houimel et al., 2001; Wyzgol et al., 2009; Borghouts et al., 2005, J. Peptide Science 11: 713-26). The positive and negative control (scrambled peptides) constructs are packaged and transduced in the complete upgraded NCI-60 cell line panel. Puromycin selection, time course, and growth conditions are optimized, and the cytotoxic activity of control constructs is measured using a sulforhodamine B (SRB) assay. Cell lines with poor growth characteristics, high spontaneous cell death (with negative control constructs), heterogeneity, or a poor response to the expression of positive control cytotoxic constructs are excluded.

For conducting the primary viability screen, 10×10⁶ cells from each cell line validated as described above is infected at MOI=0.3-0.5 in six replicates with a packaged 50K BASP lentiviral library. All cells are treated with puromycin (the lentiviral vector contains a puromycin resistance marker) to select transduced cells, and cells from three replicates are collected at 2 days post-transduction and used as a control. The remaining three cell replicates are grown at a low density (5×10⁴ cells/cm²) for 1.5-2 weeks to allow the cells that express toxic peptides to develop lethal or growth-inhibitory phenotypes induced by an autocrine mechanism involving the secreted dendrimeric peptides. Genomic DNA is isolated from the control and experimental cells, and the representation of peptide constructs is determined by HT sequencing (15×10⁶ reads per sample with the GexSeq primer; FIG. 23) of the copy number of peptide inserts rescued by PCR from genomic DNA using Gex1 and Gex2 flanking primers (FIG. 23) using the Solexa-Illumina platform (San Diego, Calif.). The cytotoxic and cytostatic peptides are identified by a decrease in the abundance level in the cells grown for 2 weeks as compared to the transduced control cells. Statistical analyses of these data are performed using SPSS v17. Positive and negative control constructs incorporated in the 50K BASP library are used to statistically estimate the reliability of depletion of cytotoxic peptide construct copy numbers.

The complete set of cytotoxic BASP hits that are identified in the primary screen (approximately 1,000 expected) are subjected to an additional round of confirmation screening with the goal of confirming the primary hits and mapping the minimum cytotoxic motif sequences. 20K-50K BASP hit sub-libraries comprising all of the primary hits and a redundant set (˜10-50 constructs/hit) of all possible deletion mutants (both N-terminal and C-terminal mutants that maintain a constant distance of the peptide from the LeuZip domain) of 4-20 amino acid peptide sequences are constructed. The 50K BASP hit sub-library is subjected to an additional round of viability screening (in triplicate) in a pooled format with the minimum most informative subset of three to five cell lines used in the primary screen. HT sequencing data is analyzed to confirm and map the minimum cytotoxic sequence motifs.

The biological activity of the confirmed hits is enhanced using a saturation scanning mutagenesis strategy. An additional 50K BASP mutant sub-library comprising all of the possible single scanning mutants (70-380 mutants per motif) in the minimum bioactive motifs revealed in the confirmation screen is prepared. To optimize the spacing between the cytotoxic motifs, additional constructs are included in the 50K mutant sub-library with different linker lengths (4-20 amino acids) that separate the peptides from the LeuZip domain. The 50K BASP mutant sub-library is used in viability screens (in triplicate) with the three to five most informative cancer cell lines. The depletion data of cytotoxic peptide mutants generated by HT sequencing is analyzed using structure-activity relationship analysis (SAR) with the goal of identifying the structures of the most active cytotoxic peptide motifs.

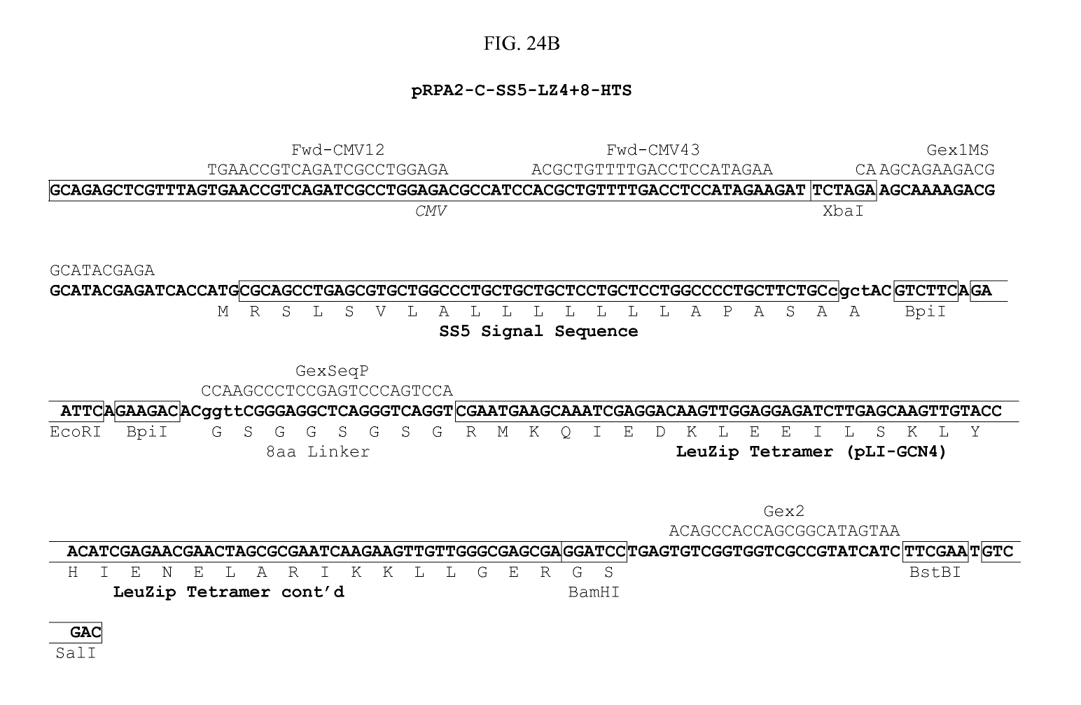

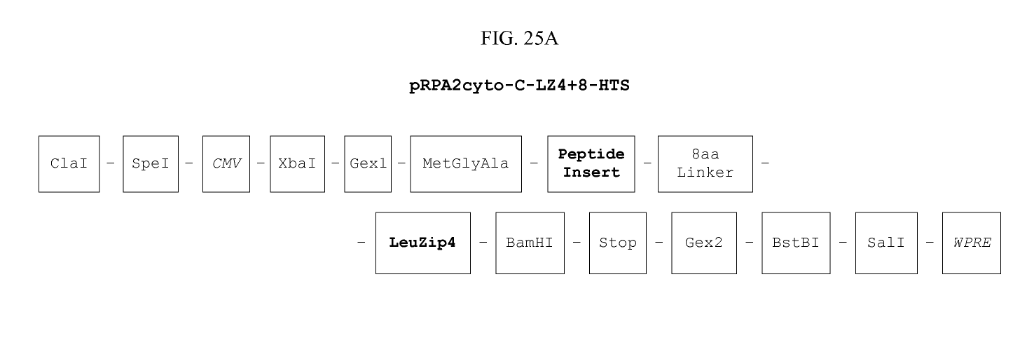

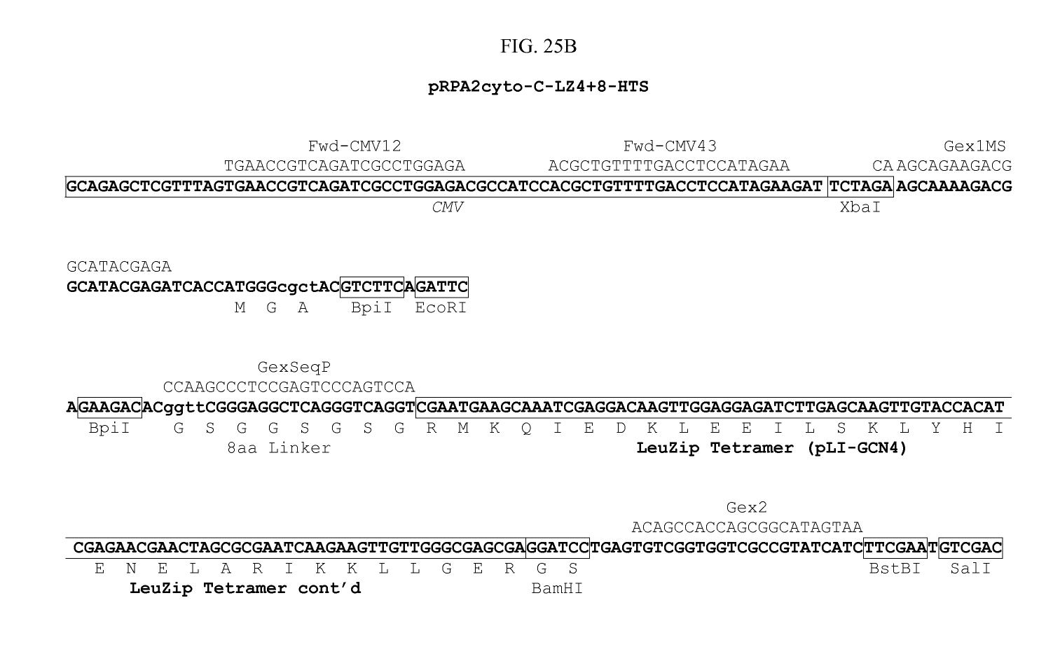

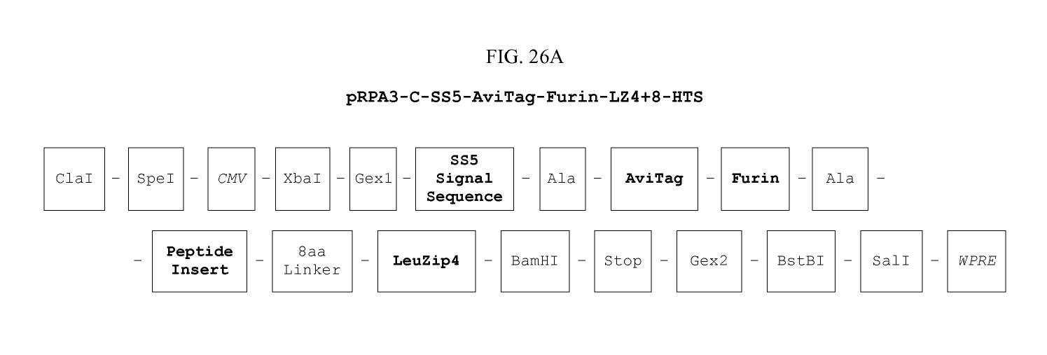

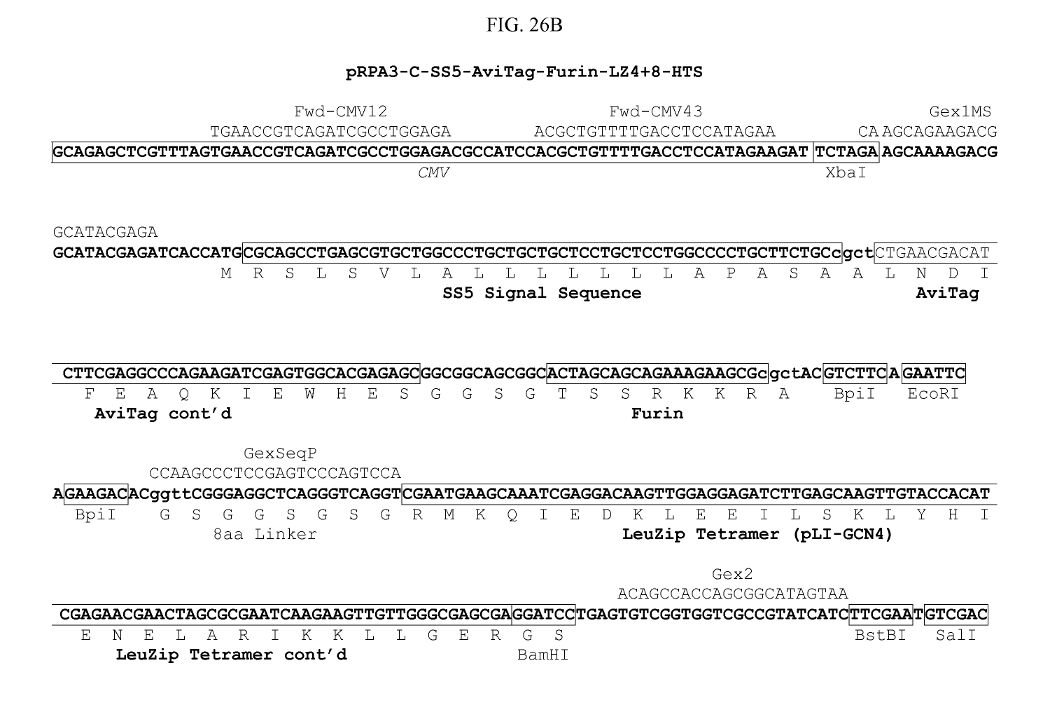

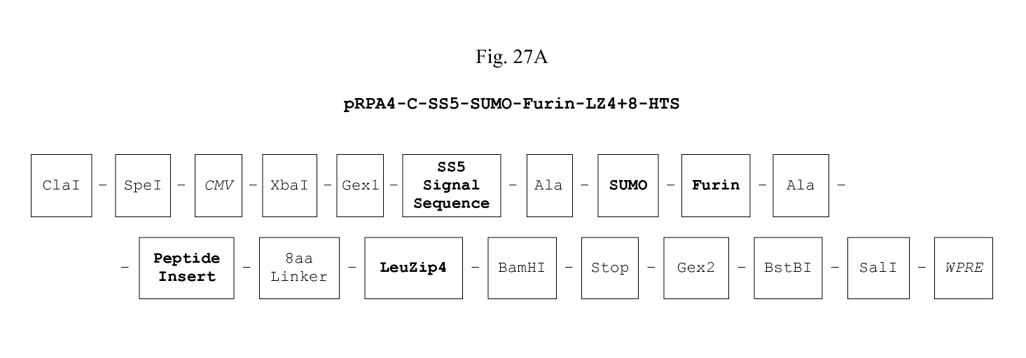

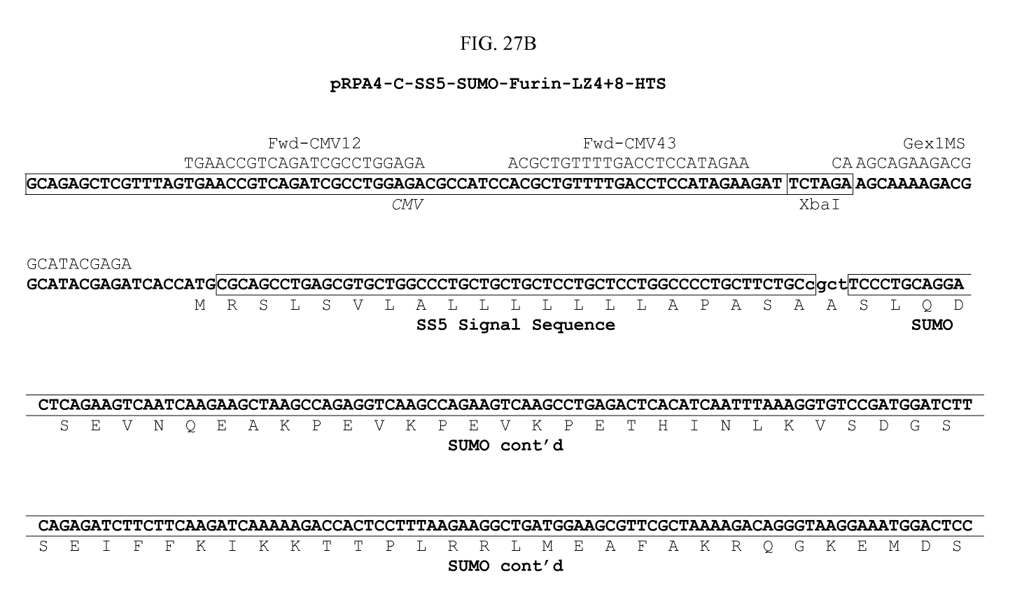

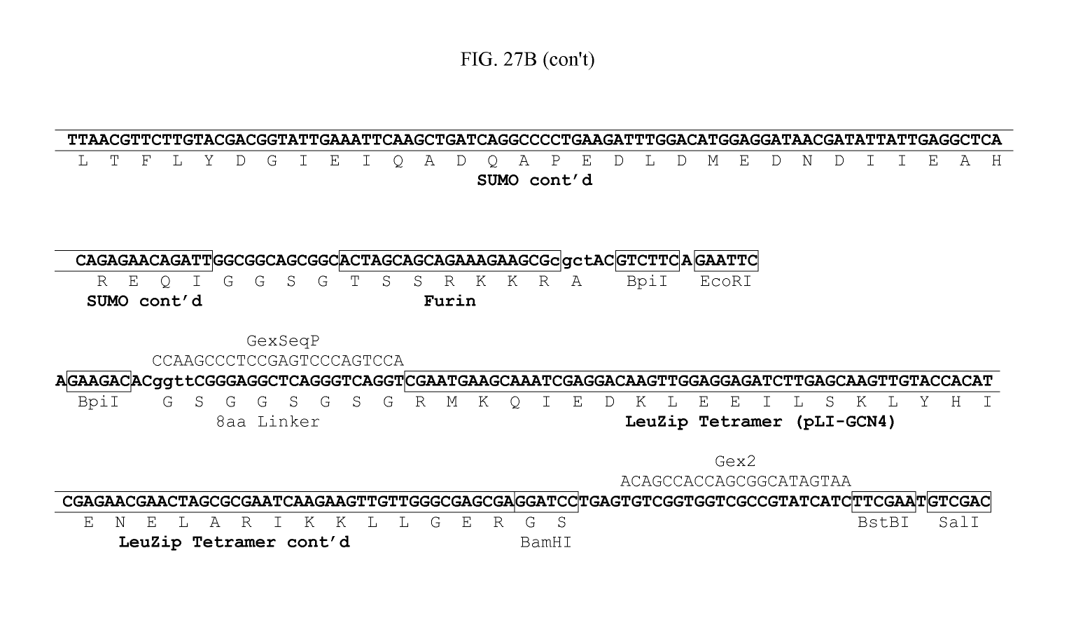

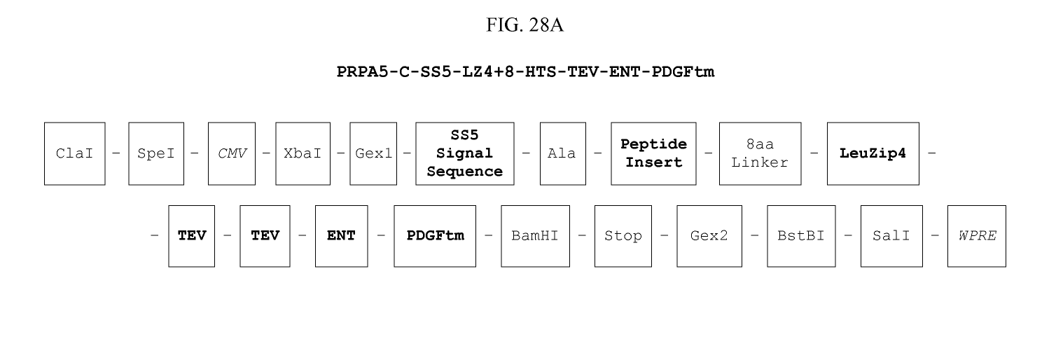

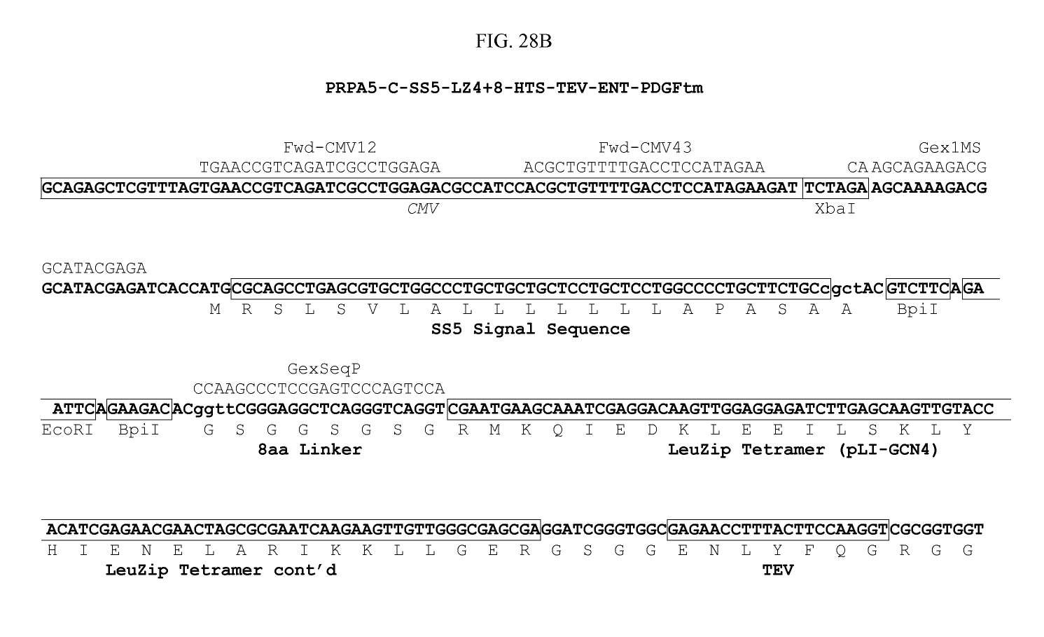

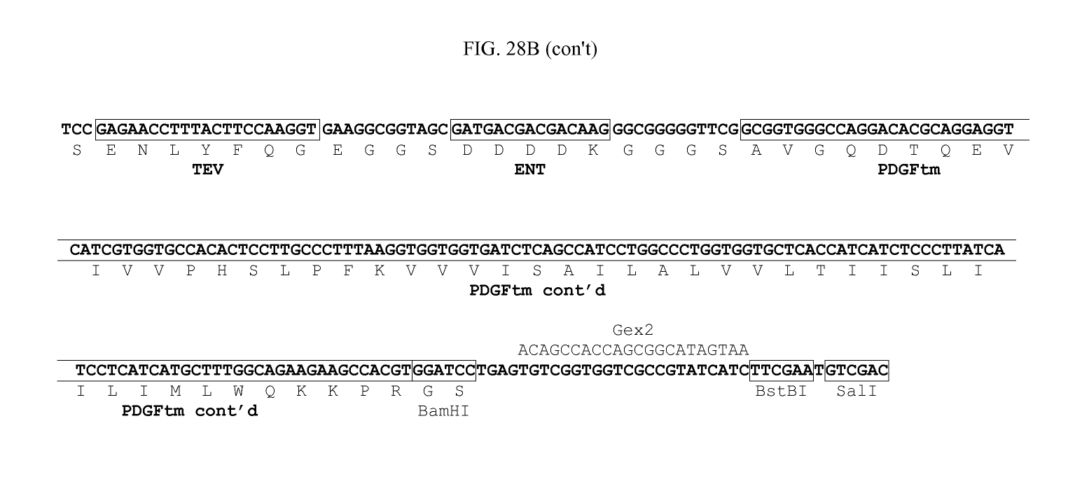

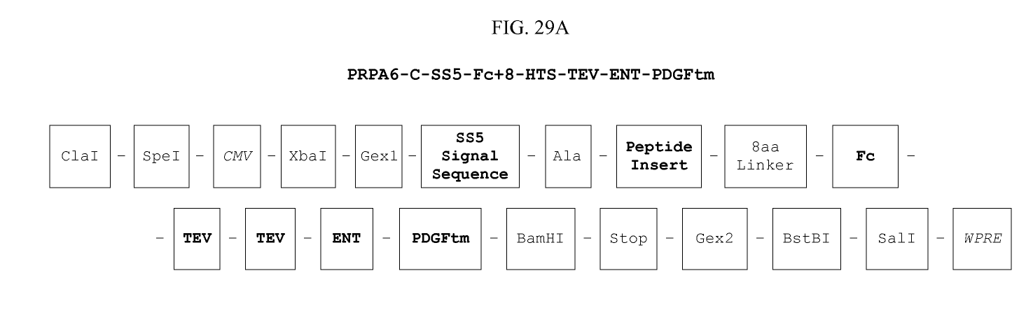

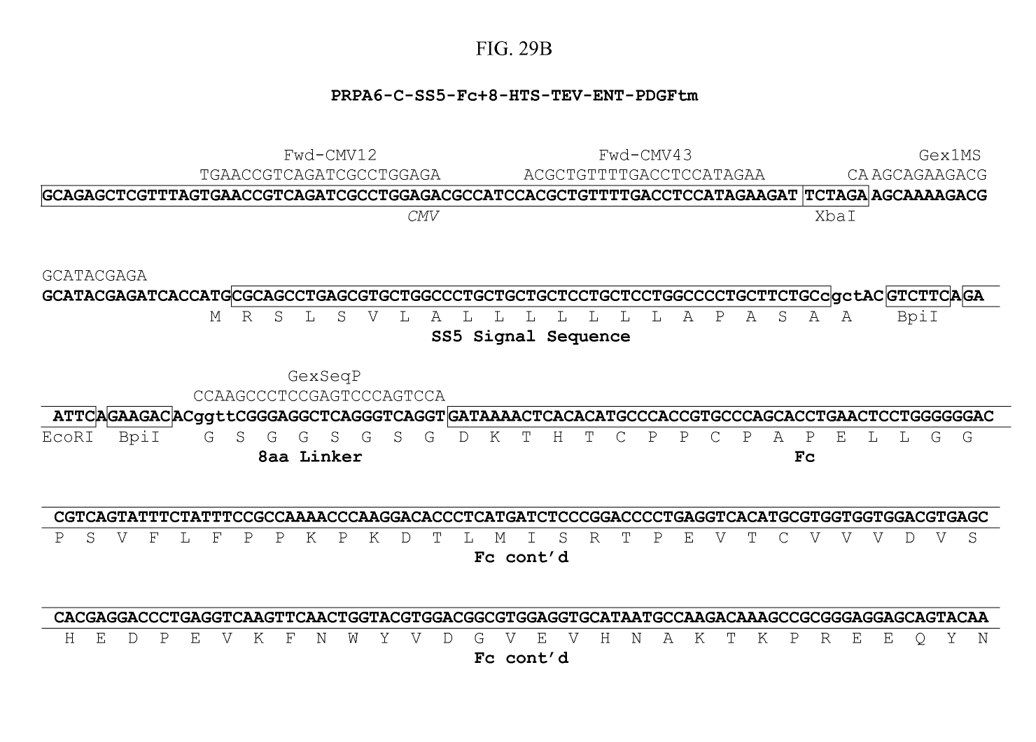

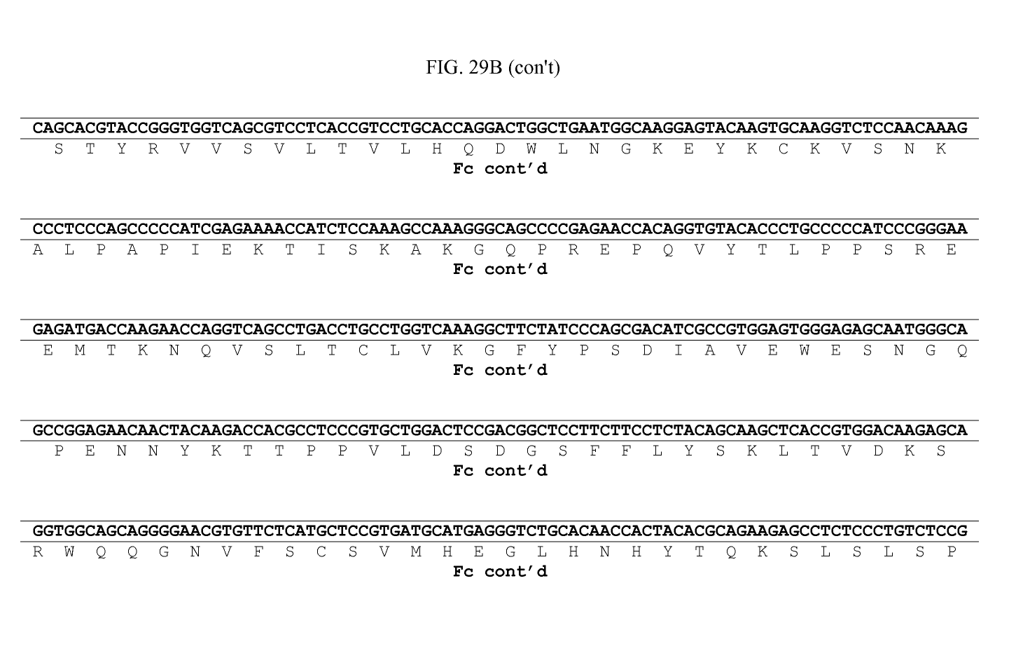

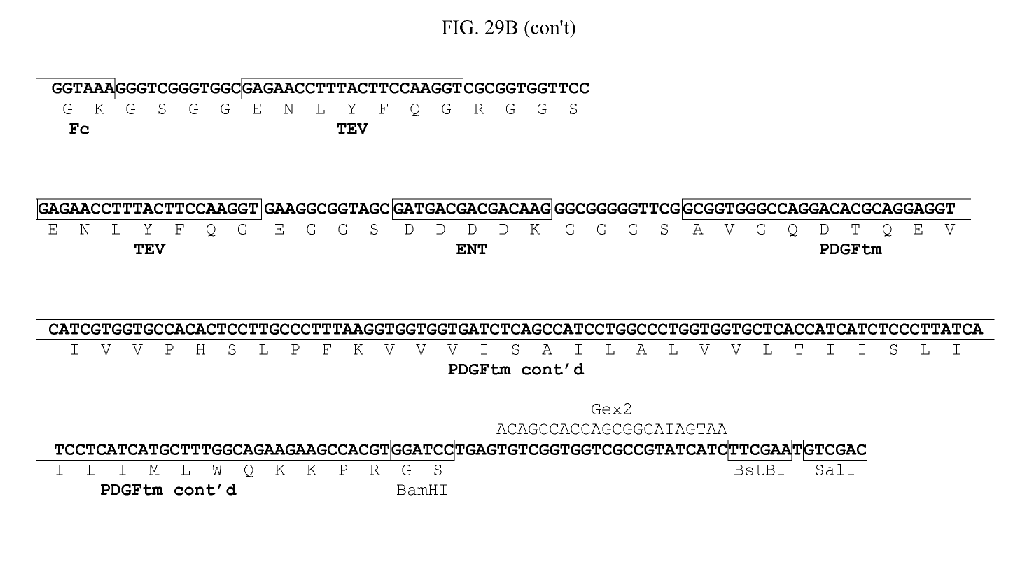

Other constructs and sequences that can be used in the reagents and methods of the invention are shown in FIGS. 24-29 and in Tables 4-7 below.

TABLE 4

StrepPep control constructs for monitoring transport
of peptides in different cell compartments.

| | |
|---|---|
| Construct | Nucleotide and Amino Acid Sequences |

| | |
|---|---|
| G1s | ATGCGCAGCC TGAGCGTGCT GGCCCTGCTG CTGCTCCTGC TCCTGGCCCC |
| | TGCTTCTGCC GCTTGGAGTC ATCCCCAGTT CGAGAAAGGC GGCGGCACTG |
| | GCGGCGGCTC AGGTGGTGGT TCGGGTTCGG GAGGCTCAGG GTCAGGTCGA |
| | ATGAAGCAAA TCGAGGACAA GTTGGAGGAG ATCTTGAGCA AGTTGTACCA |
| | CATCGAGAAC GAACTAGCGC GAATCAAGAA GTTGTTGGGC GAGCGAGGAT |
| | CCTGA |
| | [SEQ ID NO: 178] |
| | MRSLSVLALL LLLLLAPASA AWSHPQFEKG GGTGGGSGGG SGSGGSGSGR |
| | MKQIEDKLEE ILSKLYHIEN ELARIKKLLG ERGS |
| | [SEQ ID NO: 179] |
| | Key: |
| | SS5 - StrepPep - L8 - LZ4 - BamHI |
| G1sCyto | ATGGGCGCTT GGAGTCATCC CCAGTTCGAG AAAGGCGGCG GCACTGGCGG |
| | CGGCTCAGGT GGTGGTTCGG GTTCGGGAGG CTCAGGGTCA GGTCGAATGA |
| | AGCAAATCGA GGACAAGTTG GAGGAGATCT TGAGCAAGTT GTACCACATC |
| | GAGAACGAAC TAGCGCGAAT CAAGAAGTTG TTGGGCGAGC GAGGATCCTGA |
| | [SEQ ID NO: 180] |
| | MGAWSHPQFE KGGGTGGGSG GSGSGGSGSG RMKQIEDKLE EILSKLYHIE |
| | NELARIKKLL GERGS |
| | [SEQ ID NO: 181] |
| | Key: |
| | StrepPep - L8 - LZ4 - BamHI |
| G1f | MRSLSVLALL LLLLLAPASA ADYKDDDDKG GGTGGGSGGG SGSGGSGSGR |
| | MKQIEDKLEE ILSKLYHIEN ELARIKKLLG ERGS |
| | [SEQ ID NO: 182] |
| | Key: |
| | SS5 - FlagPep - L8 - LZ4 - BamHI |
| Ex1s | ATGCGCAGCC TGAGCGTGCT GGCCCTGCTG CTGCTCCTGC TCCTGGCCCC |
| | TGCTTCTGCC GCTCTGAACG ACATCTTCGA GGCCCAGAAG ATCGAGTGGC |
| | ACGAGAGCGG CGGCAGCGGC ACTAGCAGCA GAAAGAAGCG CGCTTGGAGT |
| | CATCCCCAGT TCGAGAAAGG CGGCGGCACT GGCGGCGGCT CAGGTGGTGG |
| | TTCGGGTTCG GGAGGCTCAG GGTCAGGTCG AATGAAGCAA TCGAGGACAA |
| | GTTGGAGGAG ATCTTGAGCA AGTTGTACCA CATCGAGAAC GAACTAGCGC |
| | GAATCAAGAA GTTGTTGGGC GAGCGAGGAT CCTGA |
| | [SEQ ID NO: 183] |
| | codon-optimized nucleotide sequence: |
| | ATGCGCAGCC TGAGCGTGCT GGCCCTGCTG CTGCTCCTGC TCCTGGCCCC |
| | TGCTTCTGCG GCGCTGAACG ACATCTTCGA GGCCCAGAAG ATCGAGTGGC |
| | ACGAGAGCGG CGGCAGCGGC ACTAGCAGCA GAAAGAAGAG AGCATGGAGT |
| | CATCCCCAGT TCGAGAAAGG CGGCGGCACT GGCGGCGGCT CAGGTGGTGG |
| | TTCGGGTTCG GGAGGCTCAG GGTCAGGTCG AATGAAGCAA ATCGAGGACA |
| | AGTTGGAGGA GATCTTGAGC AAGTTGTACC ACATCGAGAA CGAACTAGCG |
| | CGAATCAAGA AGTTGTTGGG CGAGCGAGGG TCGTGA |
| | [SEQ ID NO: 184] |
| | MRSLSVLALL LLLLLAPASA ALNDIFEAQK IEWHESGGSG TSSRKKRAWS |
| | HPQFEKGGGT GGGSGGGSGS GGSGSGRMKQ IEDKLEEILS KLYHIENELA |
| | RIKKLLGERG S |
| | [SEQ ID NO: 185] |
| | Key: |
| | SS5 - AviTag - Furin - StrepPep - L8 - LZ4 - BamHI |
| Ex2s | ATGCGCAGCC TGAGCGTGCT GGCCCTGCTG CTGCTCCTGC TCCTGGCCCC |
| | TGCTTCTGCC GCTTCCCTGC AGGACTCAGA AGTCAATCAA GAAGCTAAGC |
| | CAGAGGTCAA GCCAGAAGTC AAGCCTGAGA CTCACATCAA TTTAAAGGTG |
| | TCCGATGGAT CTTCAGAGAT CTTCTTCAAG ATCAAAAAGA CCACTCCTTT |
| | AAGAAGGCTG ATGGAAGCGT TCGCTAAAAG ACAGGGTAAG GAAATGGACT |
| | CCTTAACGTT CTTGTACGAC GGTATTGAAA TTCAAGCTGA TCAGGCCCCT |
| | GAAGATTTGG ACATGGAGGA TAACGATATT ATTGAGGCTC ACAGAGAACA |
| | GATTGGCGGC AGCGGCACTA GCAGCAGAAA GAAGCGCGCT TGGAGTCATC |
| | CCCAGTTCGA GAAAGGCGGC GGCACTGGCG GCGGCTCAGG TGGTGGTTCG |
| | GGTTCGGGAG GCTCAGGGTC AGGTCGAATG AAGCAAATCG AGGACAAGTT |
| | GGAGGAGATC TTGAGCAAGT TGTACCACAT CGAGAACGAA CTAGCGCGAA |
| | TCAAGAAGTT GTTGGGCGAG CGAGGATCCT GA |
| | [SEQ ID NO: 186] |
| | MRSLSVLALL LLLLLAPASA ASLQDSEVNQ EAKPEVKPEV KPETHINLKV |
| | SDGSSEIFFK IKKTTPLRRL MEAFAKRQGK EMDSLTFLYD GIEIQADQAP |
| | EDLDMEDNDI IEAHREQIGG SGTSSRKKRA WSHPQFEKGG GTGGGSGGGS |
| | GSGGSGSGRM KQIEDKLEEI LSKLYHIENE LARIKKLLGE RGS |
| | [SEQ ID NO: 187] |
| | Key: |
| | SS5 - SUMO - Furin- StrepPep - L8 - LZ4 - BamHI |
| Ex3s | MRSLSVLALL LLLLLAPASA ASDKIIHLTD DSFDTDVLKA DGAILVDFWA |
| | EWCGPCKMIA PILDEIADEY QGKLTVAKLN IDQNPGTAPK YGIRGIPTLL |
| | LFKNGEVAAT KVGALSKGQL KEFLDANLAG GSGTSSRKKR AWSHPQFEKG |
| | GGTGGGSGGG SGSGGSGSGR MKQIEDKLEE ILSKLYHIEN ELARIKKLLG |
| | ERGS |
| | [SEQ ID NO: 188] |
| | Key: |
| | SS5 - Trx - Furin - StrepPep - L8 - LZ4 - BamHI |
| M1s | ATGCGCAGCC TGAGCGTGCT GGCCCTGCTG CTGCTCCTGC TCCTGGCCCC |
| | TGCTTCTGCC GCTTGGAGTC ATCCCCAGTT CGAGAAAGGC GGCGGCACTG |
| | GCGGCGGCTC AGGTGGTGGT TCGGGTTCGG GAGGCTCAGG GTCAGGTCGA |
| | ATGAAGCAAA TCGAGGACAA GTTGGAGGAG ATCTTGAGCA AGTTGTACCA |
| | CATCGAGAAC GAACTAGCGC GAATCAAGAA GTTGTTGGGC GAGCGAGGAT |
| | CGGGTGGCGA GAACCTTTAC TTCCAAGGTC GCGGTGGTTC CGAGAACCTT |
| | TACTTCCAAG GTGAAGGCGG TAGCGATGAC GACGACAAGG GCGGGGGTTC |
| | GGCGGTGGGC CAGGACACGC AGGAGGTCAT CGTGGTGCCA CACTCCTTGC |
| | CCTTTAAGGT GGTGGTGATC TCAGCCATCC TGGCCCTGGT GGTGCTCACC |
| | ATCATCTCCC TTATCATCCT CATCATGCTT TGGCAGAAGA AGCCACGTGG |
| | ATCCTGA |
| | [SEQ ID NO: 189] |
| | MRSLSVLALL LLLLLAPASA AWSHPQFEKG GGTGGGSGGG SGSGGSGSGR |
| | MKQIEDKLEE ILSKLYHIEN ELARIKKLLG ERGSGGENLY FQGRGGSENL |
| | YFQGEGGSDD DDKGGGSAVG QDTQEVIVVP HSLPFKVVVI SAILALVVLT |
| | IISLIILIML WQKKPRGS |
| | [SEQ ID NO: 190] |
| | Key: |
| | SS5 - StrepPep - L8 - LZ4 - TEV - TEV - ENT - PDGFtm - |
| | BamHI |
| M4s | ATGCGCAGCC TGAGCGTGCT GGCCCTGCTG CTGCTCCTGC TCCTGGCCCC |
| | TGCTTCTGCC GCTTGGAGTC ATCCCCAGTT CGAGAAAGGC GGCGGCACTG |
| | GCGGCGGCTC AGGTGGTGGT TCGGGTTCGG GAGGCTCAGG GTCAGGTGAT |
| | AAAACTCACA CATGCCCACC GTGCCCAGCA CCTGAACTCC TGGGGGGACC |
| | GTCAGTATTT CTATTTCCGC CAAAACCCAA GGACACCCTC ATGATCTCCC |
| | GGACCCCTGA GGTCACATGC GTGGTGGTGG ACGTGAGCCA CGAGGACCCT |
| | GAGGTCAAGT TCAACTGGTA CGTGGACGGC GTGGAGGTGC ATAATGCCAA |
| | GACAAAGCCG CGGGAGGAGC AGTACAACAG CACGTACCGG GTGGTCAGCG |
| | TCCTCACCGT CCTGCACCAG GACTGGCTGA ATGGCAAGGA GTACAAGTGC |
| | AAGGTCTCCA ACAAAGCCCT CCCAGCCCCC ATCGAGAAAA CCATCTCCAA |
| | AGCCAAAGGG CAGCCCCGAG AACCACAGGT GTACACCCTG CCCCCATCCC |
| | GGGAAGAGAT GACCAAGAAC CAGGTCAGCC TGACCTGCCT GGTCAAAGGC |
| | TTCTATCCCA GCGACATCGC CGTGGAGTGG GAGAGCAATG GGCAGCCGGA |
| | GAACAACTAC AAGACCACGC CTCCCGTGCT GGACTCCGAC GGCTCCTTCT |
| | TCCTCTACAG CAAGCTCACC GTGGACAAGA GCAGGTGGCA GCAGGGGAAC |
| | GTGTTCTCAT GCTCCGTGAT GCATGAGGGT CTGCACAACC ACTACACGCA |
| | GAAGAGCCTC TCCCTGTCTC CGGGTAAAGG GTCGGGTGGC GAGAACCTTT |
| | ACTTCCAAGG TCGCGGTGGT TCCGAGAACC TTTACTTCCA AGGTGAAGGC |
| | GGTAGCGATG ACGACGACAA GGGCGGGGGT TCGGCGGTGG GCCAGGACAC |
| | GCAGGAGGTC ATCGTGGTGC CACACTCCTT GCCCTTTAAG GTGGTGGTGA |
| | TCTCAGCCAT CCTGGCCCTG GTGGTGCTCA CCATCATCTC CCTTATCATC |
| | CTCATCATGC TTTGGCAGAA GAAGCCACGT GGATCCTGA |
| | [SEQ ID NO: 191] |
| | MRSLSVLALL LLLLLAPASA AWSHPQFEKG GGTGGGSGGG SGSGGSGSGD |
| | KTHTCPPCPA PELLGGPSVF LFPPKPKDTL MISRTPEVTC VVVDVSHEDP |
| | EVKFNWYVDG VEVHNAKTKP REEQYNSTYR VVSVLTVLHQ DWLNGKEYKC |
| | KVSNKALPAP IEKTISKAKG QPREPQVYTL PPSREEMTKN QVSLTCLVKG |
| | FYPSDIAVEW ESNGQPENNY KTTPPVLDSD GSFFLYSKLT VDKSRWQQGN |
| | VFSCSVMHEG LHNHYTQKSL SLSPGKGSGG ENLYFQGRGG SENLYFQGEG |
| | GSDDDDKGGG SAVGQDTQEV IVVPHSLPFK VVVISAILAL VVLTIISLII |
| | LIMLWQKKPR GS |
| | [SEQ ID NO: 192] |
| | Key: |
| | SS5 - StrepPep - L8 - Fc - TEV - TEV - ENT - PDGFtm - |
| | BamHI |
| M7s | MRSLSVLALL LLLLLAPASA ALNDIFEAQK IEWHESGGSG TSSRKKRAWS |
| | HPQFEKGGGT GGGSGGGSGS GGSGSGRMKQ IEDKLEEILS KLYHIENELA |
| | RIKKLLGERG SGGENLYFQG RGGSENLYFQ GEGGSDDDDK GGGSAVGQDT |
| | QEVIVVPHSL PFKVVVISAI LALVVLTIIS LIILIMLWQK KPR |
| | [SEQ ID NO: 193] |
| | Key: |
| | SS5 - AviTag - Furin- StrepPep - L8 - LZ4 - TEV - TEV - |
| | ENT - PDGFtm |
| M10s | MRSLSVLALL LLLLLAPASA ALNDIFEAQK IEWHESGGSG TSSRKKRAWS |
| | HPQFEKGGGT GGGSGGGSGS GGSGSGDKTH TCPPCPAPEL LGGPSVFLFP |
| | PKPKDTLMIS RTPEVTCVVV DVSHEDPEVK FNWYVDGVEV HNAKTKPREE |
| | QYNSTYRVVS VLTVLHQDWL NGKEYKCKVS NKALPAPIEK TISKAKGQPR |
| | EPQVYTLPPS REEMTKNQVS LTCLVKGFYP SDIAVEWESN GQPENNYKTT |
| | PPVLDSDGSF FLYSKLTVDK SRWQQGNVFS CSVMHEGLHN HYTQKSLSLS |
| | PGKGSGGENL YFQGRGGSEN LYFQGEGGSD DDDKGGGSAV GQDTQEVIVV |
| | PHSLPFKVVV ISAILALVVL TIISLIILIM LWQKKPR |
| | [SEQ ID NO: 194] |
| | Key: |
| | SS5 - AviTag - Furin - StrepPep - L8 - Fc - TEV - TEV - |
| | ENT - PDGFtm |

TABLE 5
Reference sequences

| | |
|---|---|
| Name | Sequence |

| | |
|---|---|
| AviTag-Furin | LNDIFEAQKI EWHESGGSGT SSRKKR |
| | [SEQ ID NO: 195] |
| SUMOstar- | TCCCTGCAGG ACTCAGAAGT CAATCAAGAA GCTAAGCCAG |
| SUMO-Furin | AGGTCAAGCC AGAAGTCAAG CCTGAGACTC ACATCAATTT |
| | AAAGGTGTCC GATGGATCTT CAGAGATCTT CTTCAAGATC |
| | AAAAAGACCA CTCCTTTAAG AAGGCTGATG GAAGCGTTCG |
| | CTAAAAGACA GGGTAAGGAA ATGGACTCCT TAACGTTCTT |
| | GTACGACGGT ATTGAAATTC AAGCTGATCA GGCCCCTGAA |
| | GATTTGGACA TGGAGGATAA CGATATTATT GAGGCTCACA |
| | GAGAACAGAT T |
| | [SEQ ID NO: 196] |
| | SLQDSEVNQE AKPEVKPEVK PETHINLKVS DGSSEIFFKI |
| | KKTTPLRRLM EAFAKRQGKE MDSLTFLYDG IEIQADQAPE |
| | DLDMEDNDII EAHREQIGGS GTSSRKKR |
| | [SEQ ID NO: 197] |
| Trx(thioredoxin)- | SDKIIHLTDD SFDTDVLKAD GAILVDFWAE WCGPCKMIAP |
| Furin | ILDEIADEYQ GKLTVAKLNI DQNPGTAPKY GIRGIPTLLL |
| | FKNGEVAATK VGALSKGQLK EFLDANLAGG SGTSSRKKR |
| | [SEQ ID NO: 198] |

TABLE 6
Control tagged peptides to clone between BpiI sites

| | |
|---|---|
| Name | Sequence |

| | |
|---|---|
| StrepTagII-Pep | WSHPQFEKGG GTGGGSGGGS |
| (StrepPep) | [SEQ ID NO: 199] |
| FLAG-Pep | DYKDDDDKGG GTGGGSGGGS |
| (FlagPep)with | [SEQ ID NO: 200] |
| enterokinase | |
| cleavage site | |
| PDGF | AVGQDTQEVI VVPHSLPFKV VVISAILALV VLTIISLIIL |
| transmembrane | IMLWQKKPR |
| domain | [SEQ ID NO: 201] |
| Fc | DKTHTCPPCP APELLGGPSV FLFPPKPKDT LMISRTPEVT |
| | CVVVDVSHED PEVKFNWYVD GVEVHNAKTK PREEQYNSTY |
| | RVVSVLTVLH QDWLNGKEYK CKVSNKALPA PIEKTISKAK |
| | GQPREPQVYT LPPSREEMTK NQVSLTCLVK GFYPSDIAVE |
| | WESNGQPENN YKTTPPVLDS DGSFFLYSKL TVDKSRWQQG |
| | NVFSCSVMHE GLHNHYTQKS LSLSPGK |
| | [SEQ ID NO: 202] |
| | GACAAAACTC ACACATGCCC ACCGTGCCCA GCACCTGAAC |
| | TCCTGGGGGG ACCGTCAGTG TTCCTCTTCC CCCCAAAACC |
| | CAAGGACACC CTCATGATCT CCCGGACCCC TGAGGTCACA |
| | TGCGTGGTGG TGGACGTGAG CCACGAGGAC CCTGAGGTCA |
| | AGTTCAACTG GTACGTGGAC GGCGTGGAGG TGCATAATGC |
| | CAAGACAAAG CCGCGGGAGG AGCAGTACAA CAGCACGTAC |
| | CGTGTGGTCA GCGTCCTCAC CGTCCTGCAC CAGGACTGGC |
| | TGAATGGCAA GGAGTACAAG TGCAAGGTCT CCAACAAAGC |
| | CCTCCCAGCC CCCATCGAGA AAACCATCTC CAAAGCCAAA |
| | GGGCAGCCCC GAGAACCACA GGTGTACACC CTGCCCCCAT |
| | CCCGGGAGGA GATGACCAAG AACCAGGTCA GCCTGACCTG |
| | CCTGGTCAAA GGCTTCTATC CCAGCGACAT CGCCGTGGAG |
| | TGGGAGAGCA ATGGGCAGCC GGAGAACAAC TACAAGACCA |
| | CGCCTCCCGT GCTGGACTCC GACGGCTCCT TCTTCCTCTA |
| | CAGCAAGCTC ACCGTGGACA AGAGCAGGTG GCAGCAGGGG |
| | AACGTGTTCT CATGCTCCGT GATGCATGAG GGTCTGCACA |
| | ACCACTACAC GCAGAAGAGC CTCTCCCTGT CTCCGGGTAA A |
| | [SEQ ID NO: 203] |
| Fc cassette | codon-optimized: |
| | GATAAAACTC ACACATGCCC ACCGTGCCCA GCACCTGAAC |
| | TCCTGGGGGG ACCGTCAGTA TTTCTATTTC CGCCAAAACC |
| | CAAGGACACC CTCATGATCT CCCGGACCCC TGAGGTCACA |
| | TGCGTGGTGG TGGACGTGAG CCACGAGGAC CCTGAGGTCA |
| | AGTTCAACTG GTACGTGGAC GGCGTGGAGG TGCATAATGC |
| | CAAGACAAAG CCGCGGGAGG AGCAGTACAA CAGCACGTAC |
| | CGGGTGGTCA GCGTCCTCAC CGTCCTGCAC CAGGACTGGC |
| | TGAATGGCAA GGAGTACAAG TGCAAGGTCT CCAACAAAGC |
| | CCTCCCAGCC CCCATCGAGA AAACCATCTC CAAAGCCAAA |
| | GGGCAGCCCC GAGAACCACA GGTGTACACC CTGCCCCCAT |
| | CCCGGGAAGA GATGACCAAG AACCAGGTCA GCCTGACCTG |
| | CCTGGTCAAA GGCTTCTATC CCAGCGACAT CGCCGTGGAG |
| | TGGGAGAGCA ATGGGCAGCC GGAGAACAACTACAAGACCA |
| | CGCCTCCCGT GCTGGACTCC GACGGCTCCT TCTTCCTCTA |
| | CAGCAAGCTC ACCGTGGACA AGAGCAGGTG GCAGCAGGGG |
| | AACGTGTTCT CATGCTCCGT GATGCATGAG GGTCTGCACA |
| | ACCACTACAC GCAGAAGAGC CTCTCCCTGT CTCCGGGTAA A |
| | [SEQ ID NO: 204] |

TABLE 7
Miscellaneous oligonucleotide and amino acid sequences.

| | |
|---|---|
| Name | Nucleotide Sequence |

| | |
|---|---|
| GexSeqP | ACCTGACCCT GAGCCTCCCG AACC |
| | [SEQ ID NO: 205] |
| SS5-BES-t | CTAGAAGCAA AAGACGGCAT ACGAGATCAC CATGCGCAGC |
| | CTGAGCGTGC TGGCCCTGCT GCTGCTCCTG CTCCTGGCCC |
| | CTGCTTCTGC CGCTACGTCT TCAGAATTCT GTCGA |
| | [SEQ ID NO: 206] |
| HTS-EBBS-t | AATTCTGGAT CCTGAGTGTC GGTGGTCGCC GTATCATCTT |
| | CGAATGTCGA |
| | [SEQ ID NO: 207] |
| LZ4 + 8co-t | AATTCAGAAG ACACGGTTCG GGAGGCTCAG GGTCAGGTCG |
| | AATGAAGCAA ATCGAGGACA AGTTGGAGGA GATCTTGAGC |
| | AAGTTGTACC ACATCGAGAA CGAACTAGCG CGAATCAAGA |
| | AGTTGTTGGG CGAGCGAGGA TC |
| | [SEQ ID NO: 208] |
| StrepPep-t | CGCTTGGAGT CATCCCCAGT TCGAGAAAGG CGGCGGCACT |
| | GGCGGCGGCT CAGGTGGTGG TTCGGGTT |
| | [SEQ ID NO: 209] |
| Avi-Fur-t | CGCTCTGAAC GACATCTTCG AGGCCCAGAA GATCGAGTGG |
| | CACGAGAGCG GCGGCAGCGG CACTAGCAGC AGAAAGAAGC |
| | GCGCTACGTC TTCAGAATTC AGAAGACACG GTT |
| | [SEQ ID NO: 210] |
| Met-Linker-t | CTAGAAGCAA AAGACGGCAT ACGAGATCAC CATGGGCGCT |
| | ACGTCTTCAG AATT |
| | [SEQ ID NO: 211] |
| SUMO-Fur | CGTCTCACGC TTCCCTGCAG GACTCAGAAG TCAATCAAGA |
| | AGCTAAGCCA GAGGTCAAGC CAGAAGTCAA GCCTGAGACT |
| | CACATCAATT TAAAGGTGTC CGATGGATCT TCAGAGATCT |
| | TCTTCAAGAT CAAAAAGACC ACTCCTTTAA GAAGGCTGAT |
| | GGAAGCGTTC GCTAAAAGAC AGGGTAAGGA AATGGACTCC |
| | TTAACGTTCT TGTACGACGG TATTGAAATT CAAGCTGATC |
| | AGGCCCCTGA AGATTTGGAC ATGGAGGATA ACGATATTAT |
| | TGAGGCTCAC AGAGAACAGA TTGGCGGCAG CGGCACTAGC |
| | AGCAGAAAGA AGCGCGCTAC GTCTTCAGAA TTCAGAAGAC |
| | ACGGTTTGAG ACG |
| | [SEQ ID NO: 212] |
| PDGF-Gex | CGTCTCAGAT CGGGTGGCGA GAACCTTTAC TTCCAAGGTC |
| | GCGGTGGTTC CGAGAACCTT TACTTCCAAG GTGAAGGCGG |
| | TAGCGATGAC GACGACAAGG GCGGGGGTTC GGCGGTGGGC |
| | CAGGACACGC AGGAGGTCAT CGTGGTGCCA CACTCCTTGC |
| | CCTTTAAGGT GGTGGTGATC TCAGCCATCC TGGCCCTGGT |
| | GGTGCTCACC ATCATCTCCC TTATCATCCT CATCATGCTT |
| | TGGCAGAAGA AGCCACGTGG ATCCTGAGTG TCGGTGGTCG |
| | CCGTATCATC TTCGAA |
| | [SEQ ID NO: 213] |
| Fc-PDGF | GAATTCAGAA GACACGGTTC GGGAGGCTCA GGGTCAGGTG |
| | ATAAAACTCA CACATGCCCA CCGTGCCCAG CACCTGAACT |
| | CCTGGGGGGA CCGTCAGTAT TTCTATTTCC GCCAAAACCC |
| | AAGGACACCC TCATGATCTC CCGGACCCCT GAGGTCACAT |
| | GCGTGGTGGT GGACGTGAGC CACGAGGACC CTGAGGTCAA |
| | GTTCAACTGG TACGTGGACG GCGTGGAGGT GCATAATGCC |
| | AAGACAAAGC CGCGGGAGGA GCAGTACAAC AGCACGTACC |
| | GGGTGGTCAG CGTCCTCACC GTCCTGCACC AGGACTGGCT |
| | GAATGGCAAG GAGTACAAGT GCAAGGTCTC CAACAAAGCC |
| | CTCCCAGCCC CCATCGAGAA AACCATCTCC AAAGCCAAAG |
| | GGCAGCCCCG AGAACCACAG GTGTACACCC TGCCCCCATC |
| | CCGGGAAGAG ATGACCAAGA ACCAGGTCAG CCTGACCTGC |
| | CTGGTCAAAG GCTTCTATCC CAGCGACATC GCCGTGGAGT |
| | GGGAGGCTCA TGGGCAGCCG GAGAACAACT ACAAGACCAC |
| | GCCTCCCGTG CTGGACTCCG ACGGCTCCTT CTTCCTCTAC |
| | AGCAAGCTCA CCGTGGACAA GAGCAGGTGG CAGCAGGGGA |
| | ACGTGTTCTC ATGCTCCGTG ATGCATGAGG GTCTGCACAA |
| | CCACTACACG CAGAAGAGCC TCTCCCTGTC TCCGGGTAAA |
| | GGGTCGGGTG GCGAGAACCT TTACTTCCAA GGTCGCGGTG |
| | GTTCCGAGAA CCTTTACTTC CAAGGTGAAG GCGGTAGCGA |
| | TGACGACGAC AAGGGCGGGG GTTCGGCGGT GGGCCAGGAC |
| | ACGCAGGAGG TCATCGTGGT GCCACACTCC TTGCCCTTTA |
| | AGGTGGTGGT GATCTCAGCC ATCCTGGCCC TGGTGGTGCT |
| | CACCATCATC TCCCTTATCA TCCTCATCAT GCTTTGGCAG |
| | AAGAAGCCAC GTGGATCC |
| | [SEQ ID NO: 214] |
| Natural SEAP SS | MLGPCMLLLL LLLGLRLQLS LGIIPVEEEN PDFWNREAAE |
| Sequence | ALGA |
| | [SEQ ID NO: 215] |
| | Key: |
| | Secretion signal - Mature Protein |
| Empty vector with | MLLLLLLLGL RLQLSLGGSG GRMKQIEDKI EEILSKIYHI |
| LeuZipx3 | ENEIARIKKL IGER |
| | [SEQ ID NO: 216] |
| | Key: |
| | Secretion signal - Linker - LeuZipx3 |
| Empty vector with | MLLLLLLLGL RLQLSLGGSG SDCRTLNLSV VAVSLAVGQD |
| PDGFtm | TQEVIVVPHS LPFKVVVISA ILALVVLTII SLIILIMLWQ |
| | KKPR |
| | [SEQ ID NO: 217] |
| | Key: |
| | Secretion signal - Linker - PDGFtm |
| Vector with 20aa | MLLLLLLLGL RLQLSLGGSG GRMKQIEDKI EEILSKIYHI |
| ApoF peptide | ENEIARIKKL IGERGGASRV GRSLPTEDCE NEEKEQAVHG |
| (151-180) | [SEQ ID NO: 218] |
| | Key: |
| | Secretion signal - Linker - LeuXZipx3 - Linker - |
| | ApoF-20aa |
| Vector with 50aa | MLLLLLLLGL RLQLSLGGSG GRMKQIEDKI EEILSKIYHI |
| ApoF peptide | ENEIARIKKL IGERGGASLL AREQQSTGRV GRSLPTEDCE |
| (141-190) | NEEKEQAVHN VVQLLPGVGT FYNLGTALYG |
| | [SEQ ID NO: 219] |
| | Key: |
| | Secretion signal - Linker - LeuXZipx3 - Linker - |
| | ApoF-50aa |
| Vector with 20aa | MLLLLLLLGL RLQLSLGGSG GRMKQIEDKI EEILSKIYHI |
| cartilage matrix | ENEIARIKKL IGERGGASHQ DSRDNCPTVP NSAQEDSDG |
| protein (429-478) | [SEQ ID NO: 220] |
| | Key: |
| | Secretion signal - Linker - LeuXZipx3 - Linker - |
| | CMP-20aa |
| Vector with 50aa | MLLLLLLLGL RLQLSLGGSG GRMKQIEDKI EEILSKIYHI |
| cartilage matrix | ENEIARIKKL IGERGGASDS DQDQDGDGHQ DSRDNCPTVP |
| protein (429-478) | NSAQEDSDHD GQDACDDDDD NDGVPDSG |
| | [SEQ ID NO: 221] |
| | Key: |
| | Secretion signal - Linker - LeuXZipx3 - Linker - |
| | CMP-50aa |
| SS1-SEAP | MLLLLLLLGL RLQLSLG |
| | [SEQ ID NO: 222] |
| | CTGCTGCTGC TGCTGCTGCT GGGCCTGAGG CTACAGCTCT |
| | CCCTGGGC |
| | [SEQ ID NO: 223] |
| SS2-Secrecon 1 | MWWRLWWLLL LLLLLWPMVW Aa |
| | [SEQ ID NO: 224] |
| | ATGTGGTGGC GCCTGTGGTG GCTGCTGCTG CTGCTGCTGC |
| | TGCTGTGGCC CATGGTGTGG GCC |
| | [SEQ ID NO: 225] |
| Secrecon 2 | MRPTWAWWLF LVLLLALWAP ARG |
| | [SEQ ID NO: 226] |
| | ATGCGCCCCA CCTGGGCCTG GTGGCTGTTC CTGGTGCTGC |
| | TGCTGGCCCT GTGGGCCCCC GCCCGCGGC |
| | [SEQ ID NO: 227] |
| human Cystatin S | MAGPLRAPLL LLAILAVALA VSPAAGSS |
| | [SEQ ID NO: 228] |
| SS3- | MKLVFLVLLF LGALGLCLA |
| Lactotransferrin | [SEQ ID NO: 229] |
| (TRFL₋ HUMAN) | ATGAAGCTGG TGTTCCTGGT GCTGCTCTTC CTGGGCGCTC |
| | TGGGCCTGTG CCTGGCC |
| | [SEQ ID NO: 230] |
| Erythropoietin | MGVHECPAWL WLLLSLLSLP LGLPVLG |
| (EPO₋ HUMAN) | [SEQ ID NO: 231] |
| Human a-1- | MERMLPLLAL GLLAAGFCPA VLC |
| antichymotrypsin | [SEQ ID NO: 232] |
| precursor (ATC) | |
| SS4-Modified | MGRMLPLLAL LLLAAGFCPA VLA |
| ATC | [SEQ ID NO: 233] |
| | ATGGGCAGCA TGCTGCCCCT GCTGGCCCTG CTGCTGCTGG |
| | CCGCTGGATT CTGCCCCGCT GTGCTGGCC |
| | [SEQ ID NO: 234] |
| TNF receptor | MLGIWTLLPL VLTSVA |
| superfamily | [SEQ ID NO: 235] |
| member 6 isoform 4 | |
| Human prolactin | MNIKGSPWKG SLLLLLVSNL LLCQSVAP |
| | [SEQ ID NO: 236] |
| Osteopontin | MRLAVVCLCL FGLASC |
| | [SEQ ID NO: 237] |
| SS5-Consensus 1 | MRSLSVLALL LLLLLAPASA a |
| | [SEQ ID NO: 238] |
| | ATGCGCAGCC TGAGCGTGCT GGCCCTGCTG CTGCTCCTGC |
| | TCCTGGCCCC TGCTTCTGCC |
| | [SEQ ID NO: 239] |
| SS6-Consensus 2 | MKSLSALVLL LLLLLLPGAL Aa |
| | [SEQ ID NO: 240] |
| | ATGAAGAGCC TGAGCGCCCT GGTGCTGCTG CTGCTCCTGC |
| | TGCTCCTGCC TGGAGCCCTG GCC |
| | [SEQ ID NO: 241] |
| Consensus 3 | MRGAALVLLL LLLLLLALAL Aapvp |
| | [SEQ ID NO: 242] |
| SS7-Consensus 4 | MRGAALVLLL LLLLLLAGVL Aap |
| | [SEQ ID NO: 243] |
| | ATGCGCGGAG CTGCGCTGGT GCTGCTGCTG CTGCTCCTGC |
| | TGCTCCTGGC TGGCGTGCTG GCC |
| | [SEQ ID NO: 244] |
| Consensus 5 | MRGAALVLLL LLLLLLSPAL A |
| | [SEQ ID NO: 245] |
| Targeting to ER | ----KDEL-Stop |
| sequence at the 3′- | [SEQ ID NO: 246] |
| end (C-terminus) | |
| end | |

It should be understood that the foregoing disclosure emphasizes certain specific embodiments of the invention and that all modifications or alternatives equivalent thereto are within the spirit and scope of the invention as set forth in the appended claims.

1. A recombinant expression construct comprising a nucleic acid encoding a peptide of from 4 to 100 amino acids operatively linked to a promoter that is transcriptionally functional in a mammalian cell, wherein the construct further comprises a mammalian secretion signal sequence positioned 5′ to the peptide-encoding sequence and in the translational reading frame thereof and an oligomerization sequence positioned either between the secretion signal sequence and the peptide-encoding sequence or positioned 3′ to the peptide-encoding sequence, wherein the oligomerization sequence is in the translational reading frame of the secretion signal sequence and the peptide-encoding sequence.

2. The recombinant expression construct of claim 1, wherein the nucleic acid encodes a peptide of from 5 to 20 amino acids.

3. The recombinant expression construct of either claim 1 or 2, wherein the oligomerization sequence is a leucine zipper sequence.

4. The recombinant expression construct of claim 3, wherein the leucine zipper sequence is a dimerizing sequence.

5. The recombinant expression construct of claim 3, wherein the leucine zipper sequence is a trimerizing sequence.

6. The recombinant expression construct of claim 3, wherein the leucine zipper sequence is a tetramerizing sequence.

7. The recombinant expression construct of claim 3, wherein the leucine zipper sequence is an oligomerizing sequence.

8. The recombinant expression construct of either claim 1 or 2, wherein the peptide-encoding sequence encodes a peptide from a natural proteome.

9. The recombinant expression construct of claim 8, wherein the eukaryotic extracellular proteome is a mammalian extracellular proteome.

10. The recombinant expression construct of claim 8, wherein the eukaryotic extracellular proteome is a human extracellular proteome.

11. The recombinant expression construct of claim 2, wherein the peptide-encoding sequence encodes a bioactive peptide.

12. The recombinant expression construct of claim 2, wherein the construct comprises an adenoviral vector, an adenovirus-associated viral vector, a retroviral vector, or a lentiviral vector.

13. The recombinant expression construct of claim 2, wherein the promoter is a mammalian virus promoter.

14. The recombinant expression construct of claim 2, wherein the promoter is a mammalian promoter.

15. The recombinant expression construct of claim 13, wherein the promoter is a cytomegalovirus promoter.

16. The recombinant expression construct of claim 2, wherein the promoter is an inducible promoter.

17. The recombinant expression construct of claim 2, further comprising a post-transcriptional regulatory element positioned 3′ to the peptide-encoding sequence.

18. The recombinant expression construct of claim 2, further comprising a pro-peptide sequence positioned 3′ to the secretion signal sequence and separated from peptide-encoding sequence by a protein processing sequence, wherein the protein processing sequence is recognized by processing proteases of the furin family.

19. The recombinant expression construct of claim 2, wherein the mammalian secretion signal sequence is a secreted alkaline phosphatase signal sequence, an interleukin-1 signal sequence, a CD14 signal sequence, or consensus secretion signal MRSLSVLALLLLLLLAPASAA (SEQ ID NO: 29).

20. A plurality of recombinant expression constructs according to claim 12, wherein said peptide-encoding sequence comprises a set of at least 100 different nucleic acid sequences and is made by a method comprising:
(a) synthesizing a plurality of nucleic acid sequences on a surface of a microarray, wherein each nucleic acid sequence has a specific sequence and is synthesized in a specific location of said surface;
(b) detaching the plurality of nucleic acid sequences from the microarray;
(c) amplifying the detached plurality of nucleic acids by polymerase chain reaction; and
(d) cloning the amplified plurality of nucleic acid sequences into a vector to produce said viral recombinant expression construct.

21. A eukaryotic cell culture comprising a plurality of recombinant expression constructs according to claim 20.

22. The cell culture of claim 21, further comprising a second recombinant expression construct encoding a detectable marker protein operatively linked to a promoter regulated by interaction of a cell surface protein and a protein from the extracellular proteome.

23. The cell culture of claim 22, wherein expression in the cell of a peptide encoded by one of the plurality of recombinant expression constructs regulates expression of the detectable marker protein.

24. The cell culture of claim 19, wherein the detectable marker protein encodes a selectable biological activity.

25. The cell culture of claim 24, wherein the selectable biological activity is drug resistance.

26. The cell culture of claim 21, wherein the detectable marker protein produces a detectable signal.

27. The cell culture of claim 26, wherein the detectable marker protein is green fluorescent protein.

28. The cell culture of claim 21, wherein the cell is a mammalian cell, an avian cell, or a yeast cell.

29. The cell culture of claim 21, wherein the promoter comprising the second recombinant expression construct is responsive to p53, NF-κB, HIFlalpha, HSF-1, Ap1, a differentiation marker, or a peptide hormone.

30. The cell culture of claim 24, wherein the selectable biological activity is cell proliferation, cell death, cell growth arrest, senescence, cell size, longevity in culture, cell adhesion to a substrate, or drug and other treatment sensitivity.

31. A method for isolating a bioactive peptide from a library comprising the plurality of recombinant expression constructs, comprising the step of assaying the cell culture of claim 21 and identifying cells in said culture expressing the detectable marker.

32. A method for identifying a bioactive peptide from a library comprising a plurality of recombinant expression constructs, wherein expression of the peptide is cytotoxic, comprising:
(a) introducing into a eukaryotic cell culture the plurality of recombinant expression constructs according to claim 20;
(b) growing the culture for a time sufficient for the peptides to have a cytotoxic effect;
(c) assaying the cells of the cell culture comprising non-cytotoxic peptides; and
(d) identifying the sequences of the plurality of recombinant expression constructs absent from the plurality remaining in the cell culture.

33. The method of claim 32, wherein the cells are assayed by amplifying the peptide-encoding inserts in the cells encoded by the plurality recombinant expression constructs, sequencing the amplified peptide-encoding inserts, and identifying the sequences absent from the plurality of recombinant expression constructs remaining in the cells, wherein said absent sequences encode peptides having a cytotoxic effect.

34. A method for identifying a bioactive peptide from a library comprising a plurality of recombinant expression constructs, wherein expression of the peptide is cell growth promoting, comprising:
(a) introducing into a eukaryotic cell culture the plurality of recombinant expression constructs of claim 20;
(b) growing the culture for a time sufficient for the peptides to have a cell growth promoting effect;
(c) assaying the cells of the cell culture; and
(d) identifying the sequences of the plurality of recombinant expression constructs enriched in the plurality thereof remaining in the cell culture.

35. The method of claim 34, wherein the cells are assayed by amplifying the peptide-encoding inserts in the cells encoded by the plurality recombinant expression constructs, sequencing the amplified peptide-encoding inserts, and identifying the sequences enriched from the plurality of recombinant expression constructs remaining in the cells, wherein said enriched sequences encode peptides having a cell growth promoting effect.

36. The recombinant expression construct of claim 2, wherein the peptide-encoding sequence encodes a peptide from known bioactive proteins.

37. The recombinant expression construct of claim 2, further comprising a detectable marker protein operatively linked to mammalian or viral promoter and positioned 3′ to the peptide-encoding sequence.

38. The recombinant expression construct of claim 18, wherein the protein processing sequence is recognized by processing proteases of the furin family.
